(19) United States
(12) Patent Application Publication
(10) Pub. No.: US 2024/0368582 A1
(43) Pub. Date: Nov. 7, 2024

(54) MODIFICATION OF EPOR-ENCODING NUCLEIC ACIDS

(71) Applicant: Ensoma, Inc., Boston, MA (US)

(72) Inventors: Ashvin Reddy Bashyam, Cambridge, MA (US); Martha Ann Clark, Boston, MA (US); Pak Wai Au, Belmont, MA (US); Kush M. Parmar, Brookline, MA (US)

(21) Appl. No.: 18/285,810

(22) PCT Filed: Apr. 6, 2022
(86) PCT No.: PCT/US22/23738

§ 371 Date: Oct. 5, 2023

Related U.S. Application Data
(60) Provisional application No. 63/171,568, filed on Apr. 6, 2021.

(51) Int. Cl. C12N 15/10; A61K 38/00; C07K 14/715; C12N 9/10; C12N 9/22; C12N 15/11; C12N 15/86

(52) U.S. Cl. CPC C12N 15/102; C07K 14/715; C12N 9/1007; C12N 9/22; C12N 15/111; C12N 15/86; A61K 38/00; C07K 2319/02; C12N 2310/20; C12N 2710/10042; C12Y 201/01063

(57) ABSTRACT

Gene therapy typically modifies some, but not all, cells of a population of target cells. One approach to increasing the prevalence of modified cells includes delivering to the modified cells a gene that provides a competitive advantage (e.g., a proliferative advantage) and therefore results in enrichment of modified cells. The present disclosure including, among other things, methods and compositions for providing a competitive advantage to one or more cells by providing to the cells a nucleic acid encoding a signaling-enhanced EpoR polypeptide (e.g., a truncated EpoR or gain-of-function EpoR).

### PRIORITY APPLICATION

The present application claims the benefit of U.S. Provisional Patent Application No. 63/171,568, filed Apr. 6, 2021, the content of which is hereby incorporated by reference herein in its entirety.

### BACKGROUND

Many medical conditions are caused by genetic mutation and/or are treatable, at least in part, by gene therapy. Some conditions are particularly treatable by modification of hematopoietic stem cells (HSCs). Compositions and methods for gene therapy are therefore needed.

### SUMMARY

Gene therapy typically modifies some, but not all, cells of a population of target cells. One approach to increasing the prevalence of modified cells includes delivering to the modified cells a gene that provides a competitive advantage (e.g., a proliferative advantage) and therefore results in enrichment of modified cells. The present disclosure includes, among other things, methods and compositions for providing a competitive advantage to one or more cells by providing to the cells a nucleic acid encoding a signaling-enhanced EpoR polypeptide (e.g., a truncated EpoR or gain-of-function EpoR). For example, the present disclosure includes in vivo, in vitro, or ex vivo modification of endogenous erythropoietin receptor (EpoR)-encoding nucleic acids (also referred to herein as endogenous EpoR nucleic acids) of target cells to produce a modified EpoR nucleic acid that encodes signaling-enhanced EpoR (e.g., where the modification is produced by an editing enzyme). The present disclosure also includes providing target cells with a nucleic acid encoding a signaling-enhanced EpoR by in vivo, in vitro, or ex vivo contact with a vector comprising a nucleic acid that encodes a signaling-enhanced EpoR. The present disclosure further includes that providing to cells a nucleic acid encoding a signaling-enhanced EpoR to confer a competitive advantage can be combined with delivery of a therapeutic payload.

The present disclosure includes the recognition that therapeutically effective gene therapy based at least in part on modification of HSCs and/or erythroid progenitors to include, encode, and/or express a nucleic acid sequence encoding a therapeutic agent can require that a substantial percentage of HSCs and/or erythroid lineage cells (e.g. burst forming unit-erythroid (BFU-E), colony forming unit-erythroid (CFU-E), erythroblasts) include, encode, and/or express the therapeutic agent. The present disclosure further includes the recognition that in vivo HSC gene therapy that provides one or more HSCs and/or erythroid progenitor with a nucleic acid encoding signaling-enhanced EpoR can confer a competitive advantage to gene modified erythroid progenitors (including, e.g., BFU-E, CFU-E, and erythroblasts), which results in selective enrichment for modified cells at the erythroid progenitor through erythrocyte stages. When the gene therapy further includes a nucleic acid sequence encoding a therapeutic agent (e.g., in the same nucleic acid payload as the nucleic acid sequence that provides a nucleic acid encoding signaling-enhanced EpoR), the gene therapy results in concurrent selective enrichment for modified cells that include, encode, and/or express the therapeutic agent. Such enrichment confers an improvement in certain therapies by increasing the proportion of erythroid progenitors and red blood cells (RBCs) that include, encode, and/or express the therapeutic agent. In various embodiments, for example, the disease, disorder, or condition can be β-thalassemia or sickle cell disease, in which the fraction of therapeutically modified RBCs is directly related to phenotypic correction and/or efficacy of treatment. Similarly, in embodiments in which the therapeutic agent is a protein for secretion by modified cells for a therapeutic purpose, increasing the prevalence of therapeutically modified cells can increase the potency of gene therapy by increasing the total number of modified cells (and thus the total amount of the therapeutic agent that is produced and secreted).

Increasing the prevalence of modified cells, and/or achieving high levels of modified cells, by providing a competitive advantage to modified cells, is beneficial for a wide variety of therapeutic strategies, including without limitation in vivo stem cell gene therapy. In various embodiments, methods and compositions of the present disclosure are useful to increase the prevalence of modified HSCs and/or erythroid progenitors (including, e.g., BFU-E, CFU-E, and erythroblasts). In various embodiments, methods and compositions of the present disclosure are useful to increase the prevalence of modified cells in cell lineages derived from HSCs, including erythroid progenitors, which cell lineages can include differentiated cells. Utility of increasing the prevalence of modified cells is particularly evident where modification of a large fraction of a target cell population improves therapy and/or is required for treatment and/or a target clinical result. Moreover, achieving high levels of modified cells has been particularly historically challenging in the area of in vivo HSC gene therapy and/or erythroid progenitor gene therapy, which is among the problems solved by the present disclosure.

The presently disclosed methods of increasing the prevalence of modified cells and/or achieving high levels of modified cells further provide additional clinical advantages. For instance, using enrichment methods and compositions of the present disclosure, each administration of gene therapy vector at a given dose can result in a proportionally greater number or percentage of modified target cells. For at least this reason, gene therapy according to the enrichment methods and compositions of the present disclosure can achieve a target or reference therapeutic threshold (e.g., in number or percentage of therapeutically modified cells of a target cell population, and/or in total expression of a therapeutic agent) at a number of doses, unit dose size, or smaller total dose of vector and/or of nucleic acid payload than comparable reference methods and compositions that do not include the enrichment technology of the present disclosure. This benefit is particularly acute given that repeated dosing of a subject with a gene therapy vector is broadly regarded as undesirable and/or desirable to minimize. Decreasing the total dose and/or doses of vector required to achieve a reference therapeutic threshold can improve safety, e.g., by reducing innate immune activation or the risk thereof. Decreasing the total dose and/or doses of vector required to achieve a reference therapeutic threshold can also decrease costs (e.g., production costs) of a gene therapy by decreasing the amount of vector material required per patient. Engineering of cells to include, encode, and/or express a signaling-enhanced EpoR therefore provides a means to achieve desirable gene therapy results, e.g., increased prevalence of therapeutically modified erythroid lineage cells in a subject relative to unmodified reference cells (e.g., cells of the same type or types).

The present disclosure includes, without limitation, various means of modifying a nucleic acid encoding EpoR to produce a modified nucleic acid that encodes signaling-enhanced EpoR. In various embodiments, a nucleic acid encoding signaling-enhanced EpoR can be produced by any of: (1) a substitution; (2) a truncation (e.g., by introduction of a premature stop codon into the EpoR coding sequence); (3) a deletion; (4) a duplication; (5) an insertion; and/or (6) a combination of one or more of (1)-(5). In various embodiments, a modification that produces a nucleic acid sequence encoding signaling-enhanced EpoR can be introduced by any of the editing and/or sequence modification technologies disclosed herein, including without limitation (1) a CRISPR editing system (e.g., CRISPR/Cas9 or another CRISPR system that introduces double-stranded breaks), e.g., by introducing one or more indels that generate a frameshift or loss-of-function indel at one or more appropriate positions; (2) a base editing system, e.g., by introducing a truncating or otherwise signal-enhancing stop codon or substitution at one or more appropriate positions; (3) a prime editing system, e.g., by introducing a truncating or otherwise signal-enhancing stop codon, substitution, deletion, or insertion at one or more appropriate positions; (4) homology directed repair (e.g., via CRISPR/Cas9 or another CRISPR system that introduces double-stranded breaks), e.g., by introducing a truncating or otherwise signal-enhancing stop codon, substitution, deletion, or insertion at one or more appropriate positions; and/or (5) targeted integration (e.g., via a targeted transposase or retrotransposase), e.g., by introducing a truncating or otherwise signal-enhancing stop codon, substitution, deletion, or insertion at one or more appropriate positions.

Where cells are modified by a nucleic acid payload that includes a base editing system or prime editing system engineered to edit a nucleic acid encoding EpoR to produce a modified nucleic acid encoding signaling-enhanced EpoR, the editing system can further be “multiplexed” to target additional nucleic acid sequences, e.g., for therapeutic purposes. For example, the nucleic acid payload encoding the editing system could further include, encode, and/or express a targeting agent (e.g., an sgRNA or pegRNA) for therapeutic modification introducing, e.g., a Makassar HBB variant sequence in a sickle cell disease patient, to disrupt BCL11A binding to the HBG1/2 promoters, and/or to disrupt the BCL11A erythroid enhancer.

Where cells are modified by a nucleic acid payload that includes a heterologous transgene encoding signaling-enhanced EpoR, in various embodiments the transgene can be integrated into the host cell genome. For example, in various embodiments, a transgene encoding signaling-enhanced EpoR can be present in a nucleic acid payload in which the transgene is present in a Sleeping Beauty transposon such that Sleeping Beauty transposase can mediate integration of the transgene into a host cell genome. The nucleic acid payload that includes the transposon including the transgene can also include a non-integrating sequence that encodes an editing system such as a base editing system. The base editing system can be engineered to modify a therapeutic target, e.g., to introduce a Makassar HBB variant sequence in a sickle cell disease patient, to disrupt BCL11A binding to the HBG1/2 promoters, and/or to disrupt the BCL11A erythroid enhancer.

Where cells are modified by a nucleic acid payload that includes an editing system engineered to edit a nucleic acid encoding EpoR to produce a modified nucleic acid encoding signaling-enhanced EpoR, in various embodiments, the nucleic acid payload can further encode a therapeutic transgene. In certain embodiments, the editing system is non-integrating and the transgene is an integrating fragment. In various embodiments, the therapeutic transgene can encode, for example, a form of FVIII for the treatment of Hemophilia A.

A nucleic acid payload that provides to target cells a nucleic acid encoding a signaling-enhanced EpoR (e.g., by editing or introduction of a transgene) can be delivered to cells (e.g., HSCs and/or erythroid progenitors) by any of a variety of vectors disclosed herein, including without limitation a vector that is a viral vector (e.g., an adenovirus, AAV, lentivirus, vaccinia virus, measles virus, or herpes simplex virus vector) or a non-viral vector (e.g. cationic lipid nanoparticles, liposomes, polymeric nanoparticles).

The present disclosure further includes that cells provided with a nucleic acid encoding and/or expressing signaling-enhanced EpoR according to the present disclosure can be further modified with an additional selectable marker. For instance, cells could be modified to include, encode, and/or express a heterologous transgene encoding an O6-BG resistant form of MGMT (e.g. MGMT^{P140K}), such that the prevalence of modified cells could be further increased, e.g., by contacting the cells with a selecting agent including O6-BG and an alkylating agent. The selection of the transduced HSCs and/or erythroid progenitors following administration of the selecting agent is independent of the enhanced expansion of the erythroid progenitors resulting from expression of signaling-enhanced EpoR. Expression of an O6-BG resistant form of MGMT could be controlled by a different regulatory element than the signaling-enhanced EpoR.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Adoptive cell therapy: As used herein, “adoptive cell therapy” or “ACT” involves transfer of cells with a therapeutic activity into a subject, e.g., a subject in need of treatment for a condition, disorder, or disease. In some embodiments, ACT includes transfer into a subject of cells after ex vivo and/or in vitro engineering and/or expansion of the cells.

Affinity: As used herein, “affinity” refers to the strength of the sum total of non-covalent interactions between a particular binding agent (e.g., a viral vector), and/or a binding moiety thereof, with a binding target (e.g., a cell or cell type). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of K_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Allogeneic: As used herein, term “allogeneic” refers to any material derived from one subject which is then introduced to another subject, e.g., allogeneic HSC transplantation.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another (e.g., directly or via a linker, e.g., in a fusion polypeptide); in some embodiments, two or more entities that are physically associated with one another are not covalently linked to one another but are non-covalently associated, for example by means of hydrogen bonds, van der Waals interaction, hydrophobic interactions, magnetism, or a combination thereof.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Biological Sample: As used herein, the term “biological sample” refers to a sample obtained or derived from a biological source (e.g., a tissue, organism, or cell culture) of interest, as described herein. In some embodiments, a biological source is or includes an organism, such as an animal or human. In some embodiments, a biological sample is or includes biological tissue or fluid. In some embodiments, a biological sample can be or include cells (e.g., hematopoietic cells), tissue, or bodily fluid (e.g., blood). A biological sample can be a “primary sample” obtained directly from a biological source, or can be a “processed sample” (e.g., a sample prepared from a primary sample). A biological sample can also be referred to as a “sample.”

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.). In some embodiments, a domain is or includes a characteristic portion or characteristic sequence element. In the present disclosure, reference to a polypeptide such as an enzyme can refer to an identified enzyme or a domain thereof (e.g., a domain having an identified activity), or to a variant of either of these.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term “downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a composition (e.g., a formulation) necessary to result in a desired physiological change in a subject. Effective amounts are often administered for research purposes.

Endogenous: As used herein, an agent is “endogenous” if it is naturally present in a relevant context (e.g., in a cell or organism) and/or is not present in the context as the result of engineering. For example, a nucleic acid sequence can be referred to as “endogenous” to a cell if it is present in and/or expressed from a genomic coding sequence of the cell, e.g., a genomic sequence that has not been engineered, a genomic sequence present in the cell at the time of completion of cytokinesis, and/or a genomic sequence that is derived from a germline genome of a multicellular organism in which the cell is present or from which the cell was derived.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “recombinant” or “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means. In various instances, a fragment can alternatively be referred to as a portion.

Fusion polypeptide: As used herein, the term “fusion polypeptide” generally refers to a polypeptide including at least two segments. Typically, a polypeptide containing at least two such segments is considered to be a fusion polypeptide if the two segments are moieties that (1) are not included in nature in the same peptide, and/or (2) have not previously been linked to one another in a single polypeptide, and/or (3) have been linked to one another through action of the hand of man. A fusion polypeptide can include amino acids in addition to amino acids of two segments of the fusion polypeptide, or in addition to amino acids of the at least two segments of the polypeptide. Moieties present in a fusion polypeptide can be directly covalently associated or covalently associated via a linker. Moieties present in a fusion polypeptide can be referred to as “fused”. Fusion polypeptides can also be referred to as fusion proteins.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Heterologous: As used herein, an agent is “heterologous” if it is not naturally present in a relevant context and/or is only present in the context as the result of engineering. For example, a first nucleic acid sequence is “heterologous” to a second nucleic acid sequence if the first nucleic acid sequence is not operatively linked with the second nucleic acid sequence in nature and/or in a reference context. For instance, a polypeptide is “heterologous” to a regulatory sequence if it is encoded by nucleic acid sequence that is not operatively linked with the regulatory sequence in nature and/or in a reference context.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, a host cell is a cell that has been entered by a viral vector, e.g., a vector of the present disclosure, or a viral genome thereof, e.g., a viral genome disclosed herein. In some embodiments, an intended or potential host cell can be referred to as a target cell. In some embodiments, a cell or type of cell that is selectively entered and/or selectively transduced by a viral vector of the present disclosure can be referred to as a target cell of the viral vector. In some embodiments, a host cell that has been entered and/or transduced (e.g., selectively entered and/or selectively transduced) by a viral vector of the present disclosure can be referred to as a target cell of the viral vector. In some embodiments, the terms “host cell” or “target cell” include progeny of a cell that has been entered and/or transduced (e.g., selectively entered and/or selectively transduced) by a viral vector of the present disclosure, e.g., progeny that include exogenous DNA sequences derived from DNA sequences introduced by the viral vector.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context). Those of skill in the art will appreciate that terms relating to nucleotides, nucleobases, and nucleosides are related and in some instances are used interchangeably to refer to components of nucleic acids as appropriate in a provided context. For the avoidance of doubt, the term nucleic acid as used herein can refer to one or both of a DNA molecule (e.g., a single-stranded or double-stranded DNA molecule, such as genomic DNA) and an RNA molecule (e.g., a single-stranded or double-stranded RNA molecule) such as an mRNA transcript.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked”′ to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may be or include of natural amino acids, non-natural amino acids, or both. In some embodiments, a polypeptide may be or include only natural amino acids or only non-natural amino acids. In some embodiments, a polypeptide can include D-amino acids, L-amino acids, or both. In some embodiments, a polypeptide may include only L-amino acids. In some embodiments, a polypeptide may include one or more pendant groups or other modifications, e.g., one or more amino acid side chains, e.g., at the polypeptide's N-terminus, at the polypeptide's C-terminus, at non-terminal amino acids, or at any combination thereof. In some embodiments, such pendant groups or modifications may be selected from acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion.

In some embodiments, the term “polypeptide” may be appended to a name of a reference polypeptide, activity, or structure to indicate a class of polypeptides that share a relevant activity or structure. For such classes, the present specification provides and/or those skilled in the art will be aware of exemplary polypeptides within the class whose amino acid sequences and/or functions are known. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that can in some embodiments be or include a characteristic sequence element) that shows very high sequence identity, often greater than 90% or even 95%, 96%, 97%, 98%, or 99%. Such a conserved region usually encompasses at least 3-4 and in some instances up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide can be or include a fragment of a parent polypeptide. In some embodiments, a useful polypeptide may be or include a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

For the avoidance of doubt, where a polypeptide (or a nucleic acid sequence encoding a polypeptide) is referred to by a particular name, which particular name is in some instances associated with one or more particular reference sequences, those of skill in the art will appreciate that the particular name can include and be used to refer to both the particular reference sequences and to variants thereof (e.g., variants having at least 80%, 8%, 90%, 91%, 92%, 93%, 94%, 95%, 96%, 97%, 98%, 99%, 99.5%, or 99.9% identity to one or more of the reference sequences).

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof. Without wishing to be bound by any particular embodiment(s), in various embodiments a reference sequence can be a sequence associated with a sequence accession number provided herein, certain of which sequences associated with sequence accession numbers are provided in the below listing of accession sequences.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

Variant: As used herein, the term “variant” refers to an entity that shows significant structural identity with a reference entity but differs structurally from the reference entity in the presence, absence, or level of one or more chemical moieties as compared with the reference entity. In some embodiments, a variant also differs functionally from its reference entity. In various embodiments, a variant can be referred to as a “modified” form of a reference entity. In general, whether a particular entity is properly considered to be a “variant” of a reference entity is based on its degree of structural identity with the reference entity. A variant can be a molecule comparable, but not identical to, a reference. For example, a variant nucleic acid can differ from a reference nucleic acid at one or more differences in nucleotide sequence. In some embodiments, a variant nucleic acid shows an overall sequence identity with a reference nucleic acid that is at least 85%, 86%, 87%, 88%, 89%, 90%, 91%, 92%, 93%, 94%, 95%, 96%, 97%, or 99%. In many embodiments, a nucleic acid of interest is considered to be a “variant” of a reference nucleic acid if the nucleic acid of interest has a sequence that is identical to that of the reference but for a small number of sequence alterations at particular positions. In some embodiments, a variant has 10, 9, 8, 7, 6, 5, 4, 3, 2, or 1 substituted residue(s) as compared with a reference. In some embodiments, a variant has not more than 5, 4, 3, 2, or 1 residue additions, substitutions, or deletions as compared with the reference. In various embodiments, the number of additions, substitutions, or deletions is fewer than about 25, about 20, about 19, about 18, about 17, about 16, about 15, about 14, about 13, about 10, about 9, about 8, about 7, about 6, and commonly are fewer than about 5, about 4, about 3, or about 2 residues.

### BRIEF DESCRIPTION OF THE DRAWING

FIG. 1 is a pair of schematics showing (top) a human complementary DNA (cDNA) encoding wild type EpoR and (bottom) a wild type EpoR polypeptide. The cDNA schematic includes demarcation of exon boundaries. The polypeptide schematic includes demarcation of certain domains and certain amino acids, as well as the interaction of certain amino acids and domains with signaling molecules such as CIS, SOCS3, and SHP-1.

FIG. 2 is a chart showing percent edited alleles over time for EpoR W439 stop codon alleles (“TAA”, “TGA”, and “TAG”) generated by cytosine base editing targeted using Guide 1. Results are shown for each stop codon allele individually and for the stop codon alleles as a group (“STOP”). Mean and standard deviation of three replicates are shown. Data represents growth advantage conferred by the modified EpoR alleles.

FIG. 3 is a chart showing percent edited alleles over time for EpoR W439 stop codon alleles (“TAA”, “TGA”, and “TAG”) generated by cytosine base editing targeted using Guide 2. Results are shown for each stop codon allele individually and for the stop codon alleles as a group (“STOP”). Mean and standard deviation of three replicates are shown. Data represents growth advantage conferred by the modified EpoR alleles.

FIG. 4A is a chart showing fold change in allele frequency at various time points (“% tₙ”) relative to allele frequency at baseline (“% t₀”; 4 days post-transfection), over time for EpoR W439 stop codon alleles (“TAA”, “TGA”, and “TAG”) generated by cytosine base editing targeted using Guide 1. Results are shown for each stop codon allele individually and for the stop codon alleles as a group (“STOP”). Results for WT allele is shown as a reference. Mean and standard deviation of three replicates are shown. Data represents growth advantage conferred by the modified EpoR alleles.

FIG. 4B is based on the same data set as FIG. 4A and shows results for the EpoR W439 stop codon alleles as a group (“STOP”). Results for WT allele is shown as a reference. Mean and standard deviation of three replicates are shown. ** indicates a p-value≤0.05 as determined by a Student's t-test. Data represents growth advantage conferred by the modified EpoR alleles.

FIG. 5A is a chart showing fold change in allele frequency at various time points (“% tₙ”) relative to allele frequency at baseline (“% t₀”; 4 days post-transfection), over time for EpoR W439 stop codon alleles (“TAA”, “TGA”, and “TAG”) generated by cytosine base editing targeted using Guide 2. Results are shown for each stop codon allele individually and for the stop codon alleles as a group (“STOP”). Results for WT allele is shown as a reference. Mean and standard deviation of three replicates are shown. Data represents growth advantage conferred by the modified EpoR alleles.

FIG. 5B is based on the same data set as FIG. 5A and shows results for the EpoR W439 stop codon alleles as a group (“STOP”). Results for WT allele is shown as a reference. Mean and standard deviation of three replicates are shown. ** indicates a p-value≤0.05 as determined by a Student's t-test. Data represents growth advantage conferred by the modified EpoR alleles.

### DETAILED DESCRIPTION

The present disclosure includes, among other things, in vivo, in vitro, and or ex vivo modification of cells to provide the cells with a nucleic acid sequence encoding a signaling-enhanced endogenous erythropoietin receptor (EpoR), and the recognition that such modification is useful in in vivo, in vitro, and or ex vivo gene therapy. Erythropoietin (Epo) is the major regulator of red blood cell development. It inhibits the apoptosis of late erythroid progenitors and stimulates their proliferation. The erythropoietin receptor protein functions as a homodimer. It has been observed that hematopoietic stem cells (HSCs) and/or erythroid progenitors (including, e.g., BFU-E, CFU-E, and erythroblasts) in which the endogenous EpoR gene encodes a truncated EpoR have a competitive advantage over HSCs and/or erythroid progenitors (including, e.g., BFU-E, CFU-E, and erythroblasts) bearing a wild-type EpoR gene. Without wishing to be bound by any particular scientific theory, it has been hypothesized that certain mutations in the EpoR gene (e.g., certain truncation or gain-of-function mutations) result in modifications of EpoR signaling (e.g., reduction of one or more of SHP1 interaction, CIS interaction, and/or SOCS3 interaction) that contribute to erythropoietin hypersensitivity and erythrocytosism. As disclosed herein, such mutations can be referred to as “signaling-enhanced” in that the mutation increases the frequency, strength, and/or impact of signals impacted by EpoR that result in HSC and/or erythroid progenitor proliferation. The present disclosure includes the recognition that mammalian hematopoietic cells (e.g., human hematopoietic cells, including without limitation HSCs and/or erythroid progenitors including BFU-E, CFU-E, and erythroblasts) engineered to encode and/or express signaling-enhanced EpoR (e.g., truncated EpoR (tEpoR) or gain-of-function EpoR) are conferred a proliferative advantage (e.g., within erythroid progenitors) over reference cells (e.g., reference HSCs, reference erythroid progenitors, and/or cells derived from reference HSCs or reference erythroid progenitors) that encode a reference EpoR (i.e., encode an EpoR polypeptide that is not a signaling-enhanced EpoR polypeptide, such as a reference EpoR polypeptide provided herein). The present disclosure includes that HSCs can be contacted with a vector of the present disclosure to produce a modified HSC that encodes signaling-enhanced EpoR. The present disclosure includes that methods and compositions of the present disclosure can produce modified erythroid progenitors by modification of HSCs, where the produced modified erythroid progenitors are derived from modified HSCs (e.g., where the modified erythroid progenitors are not directly contacted with a vector of the present disclosure but are derived from modified HSCs contacted with a vector of the present disclosure).

Moreover, the present disclosure includes the recognition that, for at least this reason, cells that encode (i.e., cells that include a nucleic acid encoding) signaling-enhanced EpoR can be therapeutically useful, e.g., when the modified cells (including cells that include, encode, and/or express a nucleic acid payload of the present disclosure) are therapeutic cells and/or further modified to include a therapeutic modification (e.g., engineered to include, encode, and/or express a therapeutic payload). In various embodiments, the competitive advantage of cells encoding the signaling-enhanced EpoR can cause a concomitant increase in the prevalence of cells including a therapeutic modification, thereby enhancing the therapeutic benefit resulting from the therapeutic modification. In various embodiments, the competitive advantage of cells encoding the signaling-enhanced EpoR can cause a concomitant increase in the prevalence of therapeutic cells, thereby enhancing the therapeutic benefit resulting from the therapeutic cells. As disclosed herein, therapeutic cells can include any cells that cause, elicit, or contribute to a desired pharmacological and/or physiological effect. In various embodiments, therapeutic cells are HSCs and/or erythroid progenitors of a subject.

The present disclosure includes the recognition that various advantages are associated with in vivo, in vitro, and or ex vivo modification of cells to encode and/or express a nucleic acid encoding signaling-enhanced EpoR (e.g., a heterologous transgene encoding signaling-enhanced EpoR and/or an edited endogenous nucleic acid encoding signaling-enhanced EpoR). In particular, cells encoding signaling-enhanced EpoR, at least in part due to their competitive advantage over reference cells, can increase in prevalence in a subject or system over time. This characteristic can be utilized to therapeutic advantage where cells encoding signaling-enhanced EpoR are therapeutic cells, e.g., cells modified to encode and/or express a therapeutic agent. In this context, the present disclosure further includes the recognition that various additional advantages are associated where the benefits contemplated by the present disclosure achieved by in vivo, in vitro, and or ex vivo modification of endogenous EpoR-encoding nucleic acids. For example, expression of signaling-enhanced EpoR from in vivo, in vitro, and or ex vivo modified endogenous EpoR-encoding nucleic acids can be tuned to endogenous expression levels (e.g., in that the level of expression of signaling-enhanced EpoR polypeptides in modified cells is at most about the level of expression of endogenous EpoR in reference cells). Without wishing to be bound by any particular scientific theory, this is at least in part because expression of signaling-enhanced EpoR from in vivo, in vitro, and or ex vivo modified endogenous EpoR-encoding nucleic acids is controlled by endogenous regulatory sequences. In certain embodiments, expression of signaling-enhanced EpoR from in vivo, in vitro, and or ex vivo modified endogenous EpoR-encoding nucleic acids is associated with certain advantageous characteristics further to those advantages that characterize all methods of providing a nucleic acid encoding signaling-enhanced EpoR disclosed herein (including, without limitation, by providing a nucleic acid that encodes signaling-enhanced EpoR). For instance, transduction of cells with a nucleic acid including a transgene that encodes signaling-enhanced EpoR can result in the insertion of multiple copies and can include expression at levels that differ from those achieved by expression from endogenous regulatory sequences. Moreover, unlike systems in which a heterologous transgene encoding signaling-enhanced EpoR is inserted into a host cell genome, in vivo, in vitro, and or ex vivo modification of endogenous EpoR-encoding nucleic acids does not require potentially harmful insertion of an EpoR transgene.

In particular embodiments, cells modified to encode a signaling-enhanced EpoR are also genetically modified for an additional therapeutic purpose. In various embodiments, genetic modification for an additional therapeutic purpose can include delivery of a nucleic acid encoding a transgene and/or editing of an endogenous target nucleic acid, either or both of which can provide a therapeutic nucleic acid to a target cell. The genetic modification for the additional therapeutic purpose can provide a therapeutic nucleic acid that encodes a protein, e.g., to treat a disease, disorder, or condition. In certain embodiments, genetic modification for the additional therapeutic purpose can provide a therapeutic nucleic acid that encodes a chimeric antigen receptor (CAR), engineered T-cell receptor (TCR), checkpoint inhibitor, or therapeutic antibody. In certain embodiments, genetic modification for the additional therapeutic purpose can provide (e.g. deliver an editing system that causes) a modification of an endogenous sequence that increases expression of an endogenous globin gene.

The present disclosure includes the recognition that, using methods and compositions provided herein, a gene therapy that initially (e.g., within 1 day, 2 days, 3 days, 4 days, 5 days, 6 days, 1 week, 2 weeks, 4 weeks, 8 weeks, or 16 weeks of administration and/or prior to administration of a selection regimen) modifies a small number of cells (e.g., a number or percentage of cells insufficient to treat, substantially treat, clinically improve, substantially clinically improve, cure, and/or substantially cure a disease, disorder, or condition) can result in therapeutic efficacy and/or modification of a clinically significant number of cells (e.g., at least 1%, 2%, 3%, 4%, 5%, 10%, 15%, 20%, 25%, 30%, 40%, 50%, 60%, 70%, 80%, 90%, 91%, 92%, 93%, 94%, 95%, 96%, 97%, 98%, 99%, or 100% of target cells and/or cells of a particular subject, tissue, and/or cell population (e.g., a population of cells of a particular cell type, such as HSCs, erythroid progenitors, BFU-E, CFU-E, and/or erythroblasts)). In various embodiments, modified cells are hematopoietic cells. In various embodiments, modified cells are self-renewing, multilineage, and/or long-term repopulating cells such as HSCs or erythroid progenitors. In various embodiments, modified cells express a therapeutic payload (e.g., heterologous gene encoding a polypeptide of interest) at a therapeutically effective level.

The present disclosure includes the recognition that a therapeutically advantageous competitive advantage can be induced by introducing a heterologous transgene encoding signaling-enhanced EpoR or by editing of endogenous EpoR-encoding nucleic acids to encode signaling-enhanced EpoR, including without limitation endogenous EpoR genes encoded by cell genomes and/or endogenously expressed messenger ribonucleic acid (mRNA) molecules that encode EpoR. In various embodiments, a cell includes two endogenous copies of an EpoR gene and one or both EpoR genes are edited. In various embodiments, a cell includes one or a plurality of EpoR-encoding mRNA molecules expressed from a genomic EpoR gene and one or more of the EpoR-encoding mRNA molecules are edited. The present disclosure includes the recognition that editing of mRNA is more transient and/or reversible as compared to editing of genomic DNA. Transient modification can minimize any disruption of endogenous processes and/or associated effects on fitness (e.g., reduced long-term fitness or reduced fitness under certain conditions). The present disclosure appreciates that an EpoR variant (e.g., a truncated EpoR) expressed at endogenous levels, and in particular expressed at levels controlled and/or capped by endogenous regulatory sequences and/or mechanisms, are sufficient to confer a competitive advantage.

### I. Erythropoietin Receptor (EpoR)

Erythropoietin (Epo) is a key cytokine for erythroid proliferation and differentiation. Epo acts at least in part through its interaction with its receptor, EpoR. Human EpoR polypeptides in which the cytoplasmic domain of EpoR is truncated (thEpoR) are associated with hypersensitivity of erythroid precursors to erythropoietin, as are certain gain-of-function mutations. Clinically, inheritance of a nucleic acid that encodes signaling-enhanced EpoR causes a disorder known as primary familial congenital polycythemia. This disorder is characterized by hyperproliferation of erythrocytes (polycythemia). The present disclosure recognizes that enhanced proliferation resulting from expression of signaling-enhanced EpoR can be harnessed for therapeutic benefit, e.g., in an in vivo, ex vivo, or in vitro method of gene therapy as a mechanism to increase the prevalence of therapeutic cells.

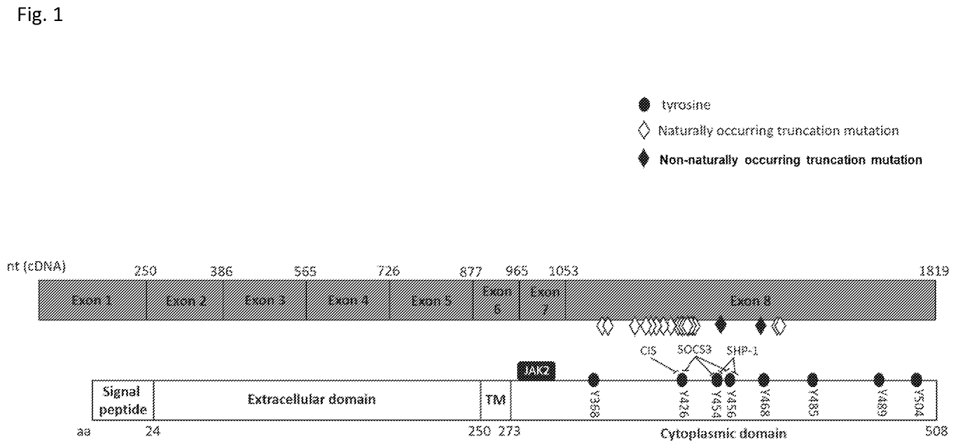

A reference EpoR polypeptide can have a sequence according to SEQ ID NO: 1 (below; see also GenBank accession no. NP_000112.1 and FIG. 1). In various embodiments, an EpoR polypeptide of the present disclosure can have at least 80% sequence identity with SEQ ID NO: 1 (e.g., 80%, 85%, 90%, 91%, 92%, 93%, 94%, 95%, 96%, 97%, 98%, 99%, or 100% sequence identity with SEQ ID NO: 1). EpoR according to SEQ ID NO: 1 can be referred to as “canonical” or “wild type” EpoR. In various embodiments, numbering of amino acids of an EpoR polypeptide (e.g., wild type EpoR or variant of EpoR, such as a truncated EpoR) can be based on numbering that corresponds to SEQ ID NO: 1.

As those of skill in the art will appreciate, genes of the human genome that encode polypeptides can include one or more exons and one or more introns. Accordingly, genomic nucleotide sequences that encode and/or express polypeptides can include a larger number of nucleotides than would be minimally required to encode the sequence of the polypeptide. In various embodiments, wild type EpoR can be encoded by a nucleic acid sequence according to GenBank Accession No. NG_021395.1 (SEQ ID NO: 2). In various embodiments, an EpoR coding sequence has at least 80% sequence identity with SEQ ID NO: 2 (e.g., 80%, 85%, 90%, 91%, 92%, 93%, 94%, 95%, 96%, 97%, 98%, 99%, or 100% sequence identity with SEQ ID NO: 2). The combined coding sequences of NG_021395.1 can be presented as a single contiguous sequence encoding EpoR, as shown in SEQ ID NO: 3 (see also FIG. 1). In various embodiments, an EpoR coding sequence has at least 80% sequence identity with SEQ ID NO: 3 (e.g., 80%, 85%, 90%, 91%, 92%, 93%, 94%, 95%, 96%, 97%, 98%, 99%, or 100% sequence identity with SEQ ID NO: 3). A nucleic acid sequence encoding EpoR according to SEQ ID NO: 2 or SEQ ID NO: 3 can be referred to as a “canonical” or “wild type” EpoR nucleic acid sequence. In various embodiments, numbering of amino acids of an EpoR polypeptide (e.g., wild type EpoR or variant of EpoR, such as a truncated EpoR) can be based on numbering that corresponds to SEQ ID NO: 2 or SEQ ID NO: 3.

Those of skill in the art will be familiar with the processes by which polypeptides encoded by genomic DNA are produced. Genomic sequences are transcribed to produce messenger ribonucleic acid molecules (mRNA) in which the coding sequence of a polypeptide is found as a single contiguous sequence. The process of transcription includes replacement of thymine nucleotides with uracil nucleotides. The present disclosure includes an EpoR mRNA sequence according to SEQ ID NO: 4. In various embodiments, an EpoR mRNA sequence has at least 80% sequence identity with SEQ ID NO: 4 (e.g., 80%, 85%, 90%, 91%, 92%, 93%, 94%, 95%, 96%, 97%, 98%, 99%, or 100% sequence identity with SEQ ID NO: 4).

SEQ ID NO: 1 (wild type EpoR polypeptide):
MDHLGASLWPQVGSLCLLLAGAAWAPPPNLPDPKFESKAALLAARGPEELLCFTERLE
DLVCFWEEAASAGVGPGNYSFSYQLEDEPWKLCRLHQAPTARGAVRFWCSLPTADTSS
FVPLELRVTAASGAPRYHRVIHINEVVLLDAPVGLVARLADESGHVVLRWLPPPETPMT
SHIRYEVDVSAGNGAGSVQRVEILEGRTECVLSNLRGRTRYTFAVRARMAEPSFGGFWS
AWSEPVSLLTPSDLDPLILTLSLILVVILVLLTVLALLSHRRALKQKIWPGIPSPESEFEGLF
TTHKGNFQLWLYQNDGCLWWSPCTPFTEDPPASLEVLSERCWGTMQAVEPGTDDEGPL
LEPVGSEHAQDTYLVLDKWLLPRNPPSEDLPGPGGSVDIVAMDEGSEASSCSSALASKPS
PEGASAASFEYTILDPSSQLLRPWTLCPELPPTPPHLKYLYLVVSDSGISTDYSSGDSQGA
QGGLSDGPYSNPYENSLIPAAEPLPPSYVACS
SEQ ID NO: 3 (wild type EpoR coding sequence):
ATGGACCACCTCGGGGCGTCCCTCTGGCCCCAGGTCGGCTCCCTTTGTCTCCTGCTC
GCTGGGGCCGCCTGGGCGCCCCCGCCTAACCTCCCGGACCCCAAGTTCGAGAGCAA
AGCGGCCTTGCTGGCGGCCCGGGGGCCCGAAGAGCTTCTGTGCTTCACCGAGCGGTT
GGAGGACTTGGTGTGTTTCTGGGAGGAAGCGGCGAGCGCTGGGGTGGGCCCGGGCA
ACTACAGCTTCTCCTACCAGCTCGAGGATGAGCCATGGAAGCTGTGTCGCCTGCACC
AGGCTCCCACGGCTCGTGGTGCGGTGCGCTTCTGGTGTTCGCTGCCTACAGCCGACA
CGTCGAGCTTCGTGCCCCTAGAGTTGCGCGTCACAGCAGCCTCCGGCGCTCCGCGAT
ATCACCGTGTCATCCACATCAATGAAGTAGTGCTCCTAGACGCCCCCGTGGGGCTGG
TGGCGCGGTTGGCTGACGAGAGCGGCCACGTAGTGTTGCGCTGGCTCCCGCCGCCTG
AGACACCCATGACGTCTCACATCCGCTACGAGGTGGACGTCTCGGCCGGCAACGGC
GCAGGGAGCGTACAGAGGGTGGAGATCCTGGAGGGCCGCACCGAGTGTGTGCTGAG
CAACCTGCGGGGCCGGACGCGCTACACCTTCGCCGTCCGCGCGCGTATGGCTGAGC
CGAGCTTCGGCGGCTTCTGGAGCGCCTGGTCGGAGCCTGTGTCGCTGCTGACGCCTA
GCGACCTGGACCCCCTCATCCTGACGCTCTCCCTCATCCTCGTGGTCATCCTGGTGCT
GCTGACCGTGCTCGCGCTGCTCTCCCACCGCCGGGCTCTGAAGCAGAAGATCTGGCC
TGGCATCCCGAGCCCAGAGAGCGAGTTTGAAGGCCTCTTCACCACCCACAAGGGTA
ACTTCCAGCTGTGGCTGTACCAGAATGATGGCTGCCTGTGGTGGAGCCCCTGCACCC
CCTTCACGGAGGACCCACCTGCTTCCCTGGAAGTCCTCTCAGAGCGCTGCTGGGGGA
CGATGCAGGCAGTGGAGCCGGGGACAGATGATGAGGGCCCCCTGCTGGAGCCAGTG
GGCAGTGAGCATGCCCAGGATACCTATCTGGTGCTGGACAAATGGTTGCTGCCCCGG
AACCCGCCCAGTGAGGACCTCCCAGGGCCTGGTGGCAGTGTGGACATAGTGGCCAT
GGATGAAGGCTCAGAAGCATCCTCCTGCTCATCTGCTTTGGCCTCGAAGCCCAGCCC
AGAGGGAGCCTCTGCTGCCAGCTTTGAGTACACTATCCTGGACCCCAGCTCCCAGCT
CTTGCGTCCATGGACACTGTGCCCTGAGCTGCCCCCTACCCCACCCCACCTAAAGTA
CCTGTACCTTGTGGTATCTGACTCTGGCATCTCAACTGACTACAGCTCAGGGGACTC
CCAGGGAGCCCAAGGGGGCTTATCCGATGGCCCCTACTCCAACCCTTATGAGAACA
GCCTTATCCCAGCCGCTGAGCCTCTGCCCCCCAGCTATGTGGCTTGCTCT
SEQ ID NO: 4 (mRNA encoding wild type EpoR):
AUGGACCACCUCGGGGCGUCCCUCUGGCCCCAGGUCGGCUCCCUUUGUCUCCUGC
UCGCUGGGGCCGCCUGGGCGCCCCCGCCUAACCUCCCGGACCCCAAGUUCGAGAG
CAAAGCGGCCUUGCUGGCGGCCCGGGGGCCCGAAGAGCUUCUGUGCUUCACCGAG
CGGUUGGAGGACUUGGUGUGUUUCUGGGAGGAAGCGGCGAGCGCUGGGGUGGGC
CCGGGCAACUACAGCUUCUCCUACCAGCUCGAGGAUGAGCCAUGGAAGCUGUGUC
GCCUGCACCAGGCUCCCACGGCUCGUGGUGCGGUGCGCUUCUGGUGUUCGCUGCC
UACAGCCGACACGUCGAGCUUCGUGCCCCUAGAGUUGCGCGUCACAGCAGCCUCC
GGCGCUCCGCGAUAUCACCGUGUCAUCCACAUCAAUGAAGUAGUGCUCCUAGACG
CCCCCGUGGGGCUGGUGGCGCGGUUGGCUGACGAGAGCGGCCACGUAGUGUUGCG
CUGGCUCCCGCCGCCUGAGACACCCAUGACGUCUCACAUCCGCUACGAGGUGGAC
GUCUCGGCCGGCAACGGCGCAGGGAGCGUACAGAGGGUGGAGAUCCUGGAGGGCC
GCACCGAGUGUGUGCUGAGCAACCUGCGGGGCCGGACGCGCUACACCUUCGCCGU
CCGCGCGCGUAUGGCUGAGCCGAGCUUCGGCGGCUUCUGGAGCGCCUGGUCGGAG
CCUGUGUCGCUGCUGACGCCUAGCGACCUGGACCCCCUCAUCCUGACGCUCUCCC
UCAUCCUCGUGGUCAUCCUGGUGCUGCUGACCGUGCUCGCGCUGCUCUCCCACCG
CCGGGCUCUGAAGCAGAAGAUCUGGCCUGGCAUCCCGAGCCCAGAGAGCGAGUUU
GAAGGCCUCUUCACCACCCACAAGGGUAACUUCCAGCUGUGGCUGUACCAGAAUG
AUGGCUGCCUGUGGUGGAGCCCCUGCACCCCCUUCACGGAGGACCCACCUGCUUC
CCUGGAAGUCCUCUCAGAGCGCUGCUGGGGGACGAUGCAGGCAGUGGAGCCGGGG
ACAGAUGAUGAGGGCCCCCUGCUGGAGCCAGUGGGCAGUGAGCAUGCCCAGGAUA
CCUAUCUGGUGCUGGACAAAUGGUUGCUGCCCCGGAACCCGCCCAGUGAGGACCU
CCCAGGGCCUGGUGGCAGUGUGGACAUAGUGGCCAUGGAUGAAGGCUCAGAAGC
AUCCUCCUGCUCAUCUGCUUUGGCCUCGAAGCCCAGCCCAGAGGGAGCCUCUGCU
GCCAGCUUUGAGUACACUAUCCUGGACCCCAGCUCCCAGCUCUUGCGUCCAUGGA
CACUGUGCCCUGAGCUGCCCCCUACCCCACCCCACCUAAAGUACCUGUACCUUGU
GGUAUCUGACUCUGGCAUCUCAACUGACUACAGCUCAGGGGACUCCCAGGGAGCC
CAAGGGGGCUUAUCCGAUGGCCCCUACUCCAACCCUUAUGAGAACAGCCUUAUCC
CAGCCGCUGAGCCUCUGCCCCCCAGCUAUGUGGCUUGCUCU

### II. Signaling-Enhanced EpoR

Various methods and compositions of the present disclosure include, provide, or are useful to produce a cell (e.g., an HSC or erythroid progenitor) that includes encodes, and/or expresses a nucleic acid encoding a signaling-enhanced EpoR polypeptide. Erythropoietin (Epo) is a key cytokine for erythroid proliferation and differentiation. Gain-of-function mutations (e.g. truncations, deletions, duplications, substitutions) of human Epo receptors have been reported as naturally occurring in patients with polycythemia. For example, mutations causing truncations of the cytoplasmic domain of EpoR result in a dominantly inherited disorder-primary familial congenital polycythemia. This disorder is characterized by increased numbers of erythrocytes (polycythemia) and by in vitro hypersensitivity of erythroid precursors to erythropoietin.

Without wishing to be bound by any particular scientific theory, an intracellular C-terminal fragment of EpoR includes a domain that exerts negative control on erythropoiesis. EpoR truncations that produce signaling-enhanced EpoR cluster (i.e., are often but not necessarily found) within a region of about 220 contiguous base pairs of exon 8, centered at about nucleotide 1250 (g) (i.e., from about nucleotide 1140 to about nucleotide 1360). Various such mutations truncate the C-terminus cytoplasmic terminal region of EpoR, which includes binding sites for negative regulators CIS, SOCS3, and SHP-1. These binding sites are absent in many truncated EpoR polypeptides. Functional studies have confirmed that disruption of the negative regulator binding sites confer hypersensitivity to Epo when transfected into a cell line model and prolonged activation of the JAK2/STAT5 pathway was evident. JAK2/STAT5 signaling plays a non-redundant role in Epo/EpoR-mediated regulation of erythropoiesis by initiating signal transduction pathways that promote cell proliferation and survival and terminal erythroid differentiation.

In various embodiments, a signaling-enhanced EpoR polypeptide is or includes truncated EpoR. In various embodiments, a truncated EpoR is characterized in that an HSC or erythroid progenitor (e.g., a BFU-E, CFU-E, or erythroblast) expressing the truncated EpoR has a proliferation rate that is greater than the proliferation rate of reference HSCs or erythroid progenitors that do not express the truncated EpoR. This increase proliferation rate of, e.g., BFU-E can lead to an accelerated rate of erythroid differentiation. In various embodiments, a truncated EpoR is characterized in that an HSC and/or erythroid progenitor expressing the truncated EpoR produces within a subject or system more direct and/or indirect progeny cells than reference HSCs and/or erythroid progenitors that do not express the truncated EpoR (e.g., over the same period of time or as measured at a particular time). In various embodiments, a signaling-enhanced EpoR polypeptide is or includes a gain-of-function EpoR. In various embodiments, a gain-of-function EpoR is characterized in that an HSC and/or erythroid progenitor expressing the gain-of-function EpoR proliferates at a rate that is greater than the proliferation rate of reference HSCs and/or erythroid progenitors that do not express the gain-of-function EpoR. In various embodiments, a gain-of-function EpoR is characterized in that an HSC and/or erythroid progenitor expressing the gain-of-function EpoR produces within a subject or system more direct and/or indirect progeny cells than reference HSCs and/or erythroid progenitors that do not express the gain-of-function EpoR (e.g., over the same period of time or as measured at a particular time).

Examples of signaling-enhanced EpoR polypeptides (including truncated EpoR and gain-of-function EpoR polypeptides) and nucleic acid sequences encoding signaling-enhanced EpoR polypeptides are provided in the following table. Except as otherwise provided herein, reference to nucleotide positions of a nucleic acid encoding a signaling-enhanced EpoR polypeptide refer to positions corresponding to the sequence of SEQ ID NO: 3. Those of skill in the art will appreciate that a nucleic acid sequence and/or nucleic acid sequence modification set forth in the following table can be present in an EpoR gene (e.g., an endogenous EpoR gene or EpoR transgene), EpoR coding sequence, and/or an EpoR mRNA, the nucleotides of any of which can be numbered according to correspondence with the sequence of SEQ ID NO: 3. Moreover, those of skill in the art will appreciate that delivery to a cell of an EpoR gene (e.g., an endogenous EpoR gene or EpoR transgene), EpoR coding sequence, and/or an EpoR mRNA as set forth in the following table can produce a cell that includes, encodes, and/or expresses a nucleic acid encoding a signaling-enhanced EpoR polypeptide. Those of skill in the art will further appreciate that each nucleic acid sequence and/or nucleic acid sequence modification set forth in the Table can be provided to a cell in the form of a transgene or by editing of an endogenous nucleic acid sequence by an editing system disclosed herein. Those of skill in the art will appreciate that where a stop codon (*) is indicated that any of the known stop codons can be used (i.e., TAG, TAA, and TGA, which can also be represented by corresponding mRNA sequences of UAG, UAA, and UGA). Where a specific amino acid change is indicated but a specific nucleic acid change is not, those of skill in the art will be readily able to ascertain the codon that encodes the reference amino acid in a reference sequence and, based on the well known human genetic code, the several codons that can be introduced to encode the modified or mutant amino acid. Table 1 specifically indicates the nucleic acid change (numbering corresponding to EpoR cDNA), the corresponding amino acid change, and exemplary approaches by which a nucleic acid payload can be engineered to introduce the designated modification into a target cell. Any of the modifications disclosed herein, including without limitation those provided in Table 1, can be provided via a transgene or by prime editing, and certain of the modifications can be introduced, e.g., by base editing. Those of skill in the art will appreciate, however, that these indications are merely exemplary and are not limiting of the gene therapy tools that can be used to introduce the indicated modifications. For example, ZFNs, TALENs, and CRISPR editing systems are not included in Table 1 but can be used to generate some or all of the indicated modifications.

TABLE 1
Exemplary Modifications of an EpoR-Encoding Nucleic Acid Sequence
that Produce a Nucleic Acid Sequence Encoding Signaling-Enhanced EpoR

| | | | Exemplary |
|---|---|---|---|
| | | Selected | base editing |
| | | Characteristics | strategies (non- |
| Nucleic acid | Amino acid | of Encoded | limiting and |
| change (cDNA) | change | Polypeptides | non-exhaustive) |
| 1142_1143del | Pro381Glnfs*2 | 127 aa truncation | |
| 1271_1272del | Phe424* | 85 aa truncation | |
| 1285del | Leu429Trpfs*24 | 57 aa truncation | |
| 1195 G > T | Glu399* | 110 aa truncation | |
| 1242_1276del35 | Ser415Hisfs*18 | 77 aa truncation | |
| 1234del | Ser412Argfs*41 | 97 aa truncation | |
| 1235C > A | Ser412* | 97 aa truncation | |
| 1249G > T | Glu417* | 92 aa truncation | |
| 1281dup | Ile428Tyrfs*17 | 65 aa truncation | |
| 1282_1289 dup 8 | Asp430Glufs*26 | 54 aa truncation | |
| 1283_1289dup | Ser432Glyfs*15 | 63 aa truncation | |
| 1288dup | Asp430Glyfs*15 | 65 aa truncation | |
| 1299_1305del | Gln434Cysfs*17 | 59 aa truncation | |
| 1300 C > T | Gln434* | 75 aa truncation | CBE |
| 1316 G > A | Trp439* | 70 aa truncation | CBE |
| 1317 G > A | Trp439* | 70 aa truncation | CBE |
| 1300dup | Gln434Profs*11 | 65 aa truncation | |
| 1311_1312del | Pro438Metfs*6 | 66 aa truncation | |
| 1252_1255del | Gly418 Profs*34 | 58 aa truncation | |
| 1273G > T | Glu425* | 84 aa truncation | |
| 1362 C > G | Tyr454* | 55 aa truncation | C-to-G base |
| | | | editor |
| 1278 C > G | Tyr426* | 83 aa truncation | C-to-G base |
| | | | editor |
| | Pro381 | 127 aa truncation | |
| | Ser382delinsGln* | | |
| | Ser382* | 127 aa truncation | |
| | Leu 452* | 57 aa truncation | |
| 1202 C > G | Ser401* | 108 aa truncation | C-to-G base |
| | | | editor |
| 1220 C > G | Ser407* | 102 aa truncation | C-to-G base |
| | | | editor |
| 1362 C > G | Tyr454* | 55 aa truncation | C-to-G base |
| | | | editor |
| 1368 C > G | Tyr456* | 53 aa truncation | C-to-G base |
| | | | editor |
| 1316_ | Trp439* | 70 aa truncation | ABE |
| 1317delinsAA | | | |
| | Asp467* | 42 aa truncation | |
| 1404C > G | Tyr468* | 41 aa truncation | C-to-G base |
| | | | editor |
| | Tyr454Phe | Modification of | |
| | | regulatory domain | |
| | Tyr426Phe; | Modification of | |
| | Tyr454Phe; | regulatory domain | |
| | Tyr456Phe | | |
| | Tyr454Phe; | Modification of | |
| | Tyr456Phe | regulatory domain | |
| | Tyr426Phe | Modification of | |
| | | regulatory domain | |
| | Tyr456Phe | Modification of | |
| | | regulatory domain | |
| | Tyr426Phe; | Modification of | |
| | Tyr456Phe | regulatory domain | |
| | Tyr426Phe; | Modification of | |
| | Tyr454Phe | regulatory domain | |
| 1277A > G; | Tyr426Cys; | Modification of | ABE |
| 1361A > G; | Tyr454Cys; | regulatory domain | |
| 1367A > G | Tyr456Cys | | |
| 1361A > G; | Tyr454Cys; | Modification of | ABE |
| 1367A > G | Tyr456Cys | regulatory domain | |
| 1277A > G; | Tyr426Cys; | Modification of | ABE |
| 1361A > G | Tyr454Cys | regulatory domain | |
| 1277A > G; | Tyr426Cys; | Modification of | ABE |
| 1367A > G | Tyr456Cys | regulatory domain | |
| 1277A > G | Tyr426Cys | Modification of | ABE |
| | | regulatory domain | |
| 1361A > G | Tyr454Cys | Modification of | ABE |
| | | regulatory domain | |
| 1367A > G | Tyr456Cys | Modification of | ABE |
| | | regulatory domain | |
| 1420 C > T | Gln474* | 35 AA truncation | CBE |
| 1429 C > T | Gln477* | 32 AA truncation | CBE |
| 1276_1368del | Tyr426_ | Modification of | |
| | Tyr456del | regulatory domain | |

> changed to;
*Stop codon; fs frame shift; del deletion; delins deletion/insertion; p. phosphorylation site; truncation size refers to the number of amino acids lost; nucleotide and amino acid modifications are presented in accordance with convention (see, e.g., Hypertext transfer protocol secure:// varnomen.hgvs.org).

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a stop codon or frame shift modification that results in a truncation corresponding to 30 to 130 C-terminal amino acids of SEQ ID NO: 1, e.g., a number of amino acids having a lower bound of 30, 35, 40, 45, 50, 55, 60, 65, 70, 75, 80, 85, 90, 95, 100, 105, 110, 115, 120, or 125 amino acids and an upper bound of 35, 40, 45, 50, 55, 60, 65, 70, 75, 80, 85, 90, 95, 100, 105, 110, 115, 120, 125, or 130 amino acids.

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a stop codon or frame shift modification that results in a truncation corresponding to 35 to 80 C-terminal amino acids of SEQ ID NO: 1, e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids.

In certain embodiments, a signaling-enhanced EpoR has at least 80% (e.g., at least 85%, 90%, 95%, 96%, 97%, 98%, 99%, or 100%) identity to a reference EpoR amino acid sequence, wherein (and/or except that) the signaling-enhanced EpoR includes a truncation corresponding to 30 to 130 C-terminal amino acids of SEQ ID NO: 1, e.g., a number of amino acids having a lower bound of 30, 35, 40, 45, 50, 55, 60, 65, 70, 75, 80, 85, 90, 95, 100, 105, 110, 115, 120, or 125 amino acids and an upper bound of 35, 40, 45, 50, 55, 60, 65, 70, 75, 80, 85, 90, 95, 100, 105, 110, 115, 120, 125, or 130 amino acids.

Those of skill in the art that will appreciate that specific mutations can be introduced, e.g., by prime editing. For example, a prime editing system could introduce a stop codon into an endogenous nucleic acid sequence encoding EpoR precisely at a desired site such as, for example, in place of the 42nd-to-last residue to generate a 42-residue truncation

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a modification that results in a substitution of one or more tyrosine amino acids corresponding to Tyr426, Tyr454, and Tyr456 of SEQ ID NO: 1 with an amino acid that is not a tyrosine, e.g., with a cysteine or phenylalanine.

In certain embodiments, a signaling-enhanced EpoR has at least 80% (e.g., at least 85%, 90%, 95%, 96%, 97%, 98%, 99%, or 100%) identity to a reference EpoR amino acid sequence, wherein (and/or except that) the signaling-enhanced EpoR includes a substitution of one or more tyrosine amino acids corresponding to Tyr426, Tyr454, and Tyr456 of SEQ ID NO: 1 with an amino acid that is not a tyrosine, e.g., with a cysteine or phenylalanine.

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a modification that results in a deletion of one or more tyrosine amino acids corresponding to Tyr426, Tyr454, and Tyr456 of SEQ ID NO: 1.

In certain embodiments, a signaling-enhanced EpoR has at least 80% (e.g., at least 85%, 90%, 95%, 96%, 97%, 98%, 99%, or 100%) identity to a reference EpoR amino acid sequence, wherein (and/or except that) the signaling-enhanced EpoR includes a deletion of one or more tyrosine amino acids corresponding to Tyr426, Tyr454, and Tyr456 of SEQ ID NO: 1.

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a modification corresponding to a modification according to SEQ ID NO: 3 selected from 1142_1143del, c. 1271_1272del, c. 1285del, 1195 G>T, c. 1242_1276del 35, c. 1234del, 1235C>A, 1249G>T, c. 1281dup, c. 1282_1289 dup 8, c. 1283_1289dup, c.1288dup, c. 1299_1305del, 1300 C>T, 1316 G>A, 1317 G>A, c.1300dup, c. 1311_1312del, c.1252_1255del, 1273G>T, 1362 C>G, and/or 1278 C>G.

In certain embodiments, a signaling-enhanced EpoR has at least 80% (e.g., at least 85%, 90%, 95%, 96%, 97%, 98%, 99%, or 100%) identity to a reference EpoR amino acid sequence, wherein (and/or except that) the signaling-enhanced EpoR includes a modification corresponding to a modification according to SEQ ID NO: 1 selected from Pro381 Gln fs*2, Phe424*, Leu 429 Trp fs*24, Glu 399*, Ser 415 His fs*18, Ser 412 Arg fs*41, Ser 412*, Glu 417*, Ile428 Tyr fs*17, Asp430 Glu fs*26, Ser432 Gly fs*15, Asp430 Gly fs*15, Gln434 Cys fs*17, Gln434*, Trp439*, Trp439*, Gln434 Pro fs *11, Pro438 Met fs*6, Gly418 Pro fs *34, Glu425*, Tyr454*, and/or Tyr426*

In certain embodiments, a nucleic acid sequence encoding signaling-enhanced EpoR has at least 80% (e.g., at least 85%, 90%, 95%, 96%, 97%, 98%, 99%, or 100%) identity to a reference nucleic acid sequence encoding EpoR, wherein (and/or except that) the nucleic acid sequence encoding signaling-enhanced EpoR includes a modification corresponding to a modification according to SEQ ID NO: 3 selected from 1202 C>G, 1220 C>G, 1362 C>G, 1368 C>G, 1316_1317delinsAA, 1404C>G, 1420 C>T, 1429 C>T, and/or c. 1276_1368del.

In certain embodiments, a signaling-enhanced EpoR has at least 80% (e.g., at least 85%, 90%, 95%, 96%, 97%, 98%, 99%, or 100%) identity to a reference EpoR amino acid sequence, wherein (and/or except that) the signaling-enhanced EpoR includes a modification corresponding to a modification according to SEQ ID NO: 1 selected from p.Pro381_Ser382delinsGln*, p.Ser382*, Leu 452*, Ser401*, Ser407*, Tyr454*, Tyr456*, Trp439*, Asp467*, Tyr468*, Tyr454Phe, Tyr426Phe; Tyr454Phe; Tyr456Phe, Tyr454Phe; Tyr456Phe, Tyr426Phe, Tyr456Phe, Tyr426Phe; Tyr456Phe, Tyr426Phe; Tyr454Phe, Tyr426Cys; Tyr454Cys; Tyr456Cys, Tyr454Cys; Tyr456Cys, Tyr426Cys; Tyr454Cys, Tyr426Cys; Tyr456Cys, Tyr426Cys, Tyr454Cys, Tyr456Cys, Gln474*, Gln477*, and/or Tyr426_Tyr456del.

The present disclosure includes the recognition that signaling-enhanced EpoR polypeptides can be produced by disruption and/or inactivation of tyrosine phosphorylation sites corresponding to amino acids Tyr426, Tyr454, and Tyr456 of SEQ ID NO: 1. In various embodiments, one or more of the amino acids corresponding to amino acids Tyr426, Tyr454, and Tyr456 of SEQ ID NO: 1 can be inactivated by deletion. In various embodiments, one or more of the amino acids corresponding to amino acids Tyr426, Tyr454, and Tyr456 of SEQ ID NO: 1 can be inactivated by substitution with a different amino acid. Modifications of amino acids corresponding to amino acids Tyr426, Tyr454, and Tyr456 of SEQ ID NO: 1 preferably preserve some or all of the protein stabilizing properties of the C-terminal amino acids of EpoR. The present disclosure includes particular embodiments including, e.g., Tyr426_Tyr456del, Tyr426Cys; Tyr454Cys; Tyr456Cys, Tyr426Phe; Tyr454Phe; and Tyr456Ph, as well as Tyr426 to Tyr456 region. The present disclosure includes the recognition that modification of one or two of amino acids corresponding to amino acids Tyr426, Tyr454, and Tyr456 of SEQ ID NO: 1 can be sufficient to produce a signaling-enhanced EpoR and/or to confer a proliferative advantage. As compared to various alternative modifications that produce a signaling-enhanced EpoR, including certain of those provided herein, that include modification of more than one, two, or three codons and/or amino acids, modification to substitute one, two, or three of amino acids corresponding to amino acids Tyr426, Tyr454, and Tyr456 of SEQ ID NO: 1 can benefit from one or more of (i) ease of production by gene editing approaches such as prime editing or base editing, (ii) decreased immunogenicity as compared to more substantially modified EpoR amino acid sequences, and/or (iii) greater retention of other EpoR biological activities as compared to more substantially modified EpoR amino acid sequences.

The present disclosure includes that in various embodiments it may be advantageous to modify one and/or both EpoR alleles present in the genome of a target cell. In certain embodiments, a modification of an endogenous nucleic acid encoding EpoR disclosed herein is heterozygous. For example, in certain embodiments, the present disclosure recognizes it can be advantageous for a target cell to retain a non-modified copy of EpoR in order to ensure maintenance of certain biological activities of EpoR, and/or to moderate proliferative signals. Those of skill in the art will appreciate that, in various embodiments, heterozygous modifications are expected to result from application of editing methods and compositions disclosed herein. In certain embodiments, a modification of an endogenous nucleic acid encoding EpoR disclosed herein is homozygous. For example, homozygous substitution of Asn487Ser (c.1460A>G) has been observed in humans. In various embodiments, the signaling-enhanced EpoR does not include the variant p. Tyr462Ter, 83 residue truncation.

Various EpoR mutations are described, e.g., in Al-Sheikh et al. (2008) PMID 18492694, Arcasoy et al. (2002) PMID 11929803, Minkov et al. (2013), O'Rourke et al. (2011) PMID 21437635, GenBank accession no. JQ821735.1, Perrota et al. (2010) PMID20700488, Kralovics et al. (1997) PMID 9292543, Watowich et al. (1999) PMID 10498627, Sokol et al. (1995) PMID 7795221, Kralovics et al. (1997) PMID 9292543, Arcasoy et al. (1997) PMID 9192789, Furukama et al. (1997) PMID 9359528, De la Chapelle et al. (1993), Percy et al. (1998) PMID 9488636, Rives et al. (2007) PMID 17488692, Pasquier et al. (2018) PMID 29269524, Pasquier et al. (2018) PMID 29269524, GenBank accession no. JQ821734.1, Petersen et al. (2004) PMID 15142125, Kralovics and Prchal et al. (2001) PMID 11559951, Chauveau et al. (2016) PMID 26010769, Kralovics et al. (1998) PMID 9649565, and Hoertner et al. (2002) PMID 12027890.

The present disclosure further recognizes that in various embodiments sequences including a C-terminal MDTVP (SEQ ID NO: 218) amino acid sequence are characterized by certain advantageous properties. Accordingly the present disclosure provides that, for any of the various signaling-enhanced EpoR polypeptides provided herein (e.g., in Table 1, in the text of this section, and/or throughout the present disclosure and claims), the signaling-enhanced EpoR polypeptide can include or be further modified to include a C-terminal MDTVP (SEQ ID NO: 218) amino acid sequence. The present disclosure therefore includes, as non-limiting examples, modifications of endogenous EpoR-encoding nucleic acids that produce a nucleic acid that encodes a signaling-enhanced EpoR that includes a C-terminal MDTVP (SEQ ID NO: 218) amino acid sequence and transgenes that encode a signaling-enhanced EpoR that includes a C-terminal MDTVP (SEQ ID NO: 218) amino acid sequence. A particular non-limiting example includes the modification of endogenous EpoR-encoding nucleic acid sequence according to the modification 1311_1312del mutation, which modification in various embodiments produces a C-terminal MDTVP (SEQ ID NO: 218) amino acid sequence. Those of skill in the art will appreciate that a transgene encoding any signaling-enhanced EpoR polypeptide of the present disclosure with (or without) a C-terminal MDTVP (SEQ ID NO: 218) amino acid sequence can be readily produced according to the techniques of molecular biology.

III. Nucleic Acid Payloads that Produce Cells Encoding Signaling-Enhanced EpoR and Optionally Further Encode a Therapeutic Agent

The present disclosure includes compositions and methods (e.g., for use in gene therapy) to produce one or more cells that include a nucleic acid encoding signaling-enhanced EpoR, and which in various embodiments further deliver to the same cells a therapeutic agent and/or therapeutic effect. As disclosed herein, a nucleic acid payload is an engineered nucleic acid that includes one or more nucleic acid sequences that include, encode, and/or express at least one agent that achieves a desired result (e.g., that contributes to a therapeutic goal). In various embodiments, methods and compositions of the present disclosure that produce or provide one or more cells that include a nucleic acid encoding signaling-enhanced EpoR include delivering to the one or more cells a nucleic acid payload, which nucleic acid payload can optionally include a therapeutic payload. The present disclosure includes various nucleic acid payloads that include, encode, and/or express signaling-enhanced EpoR or an agent that causes another nucleic acid to include, encode, and/or express signaling-enhanced EpoR, and can further include a therapeutic payload.

In various embodiments, the present disclosure includes a nucleic acid payload that encodes a signaling enhanced EpoR (a “signaling-enhanced EpoR transgene”). Those of skill in the art will appreciate that a nucleic acid sequence that encodes a signaling enhanced EpoR for a signaling-enhanced EpoR transgene can be operably linked to a regulatory sequence for expression of the signaling enhanced EpoR in cells, e.g., in HSCs and/or erythroid progenitors, and can be further operably linked with other regulatory sequences that mediate expression.

In various embodiments, the present disclosure includes a nucleic acid payload that encodes an editing agent or editing system that can modify an endogenous nucleic acid sequence encoding EpoR to produce a modified nucleic acid sequence that encodes signaling-enhanced EpoR (an “EpoR editing agent” or “EpoR editing system”). As provided herein, editing includes any targeted modification of a nucleic acid that results in a difference in nucleic acid sequence. Editing agents refer to molecules that can be delivered to a cell or system to cause or contribute to editing. An editing system refers to two or more editing agents that are together sufficient to cause editing (e.g., a base editor and a guide RNA or a prime editor and a guide RNA) or to a single editing agent alone sufficient to cause editing. Editing systems of the present disclosure can include at least one editing agent that includes an editing enzyme. An editing agent can be a fusion polypeptide that includes an editing enzyme. The present disclosure includes a variety of editing agents and editing systems capable of editing EpoR-encoding nucleic acids. As those of skill in the art will appreciate, many or all editing agents described herein can be targeted to induce particular changes using approaches known to those of skill in the art.

The present disclosure further includes that a nucleic acid payload that includes a signaling-enhanced EpoR transgene and/or an EpoR editing agent or system can further include or encode an agent for an additional therapeutic purpose. In various embodiments, a portion of a nucleic acid payload that includes, encodes, and/or expresses an expression product having a therapeutic purpose (optionally wherein the therapeutic purposes does not include and/or is not limited to causing expression of signaling enhanced EpoR) can be referred to as a “therapeutic payload,” “therapeutic gene,” “therapeutic transgene,” or “therapeutic module.” Thus, for example, a nucleic acid payload can include (i) a signaling-enhanced EpoR transgene and/or an EpoR editing agent or system and (ii) a therapeutic transgene encoding further payload expression product such as an antibody, enzyme, chimeric antigen receptor, T cell receptor, or other therapeutic agent. The present disclosure further includes embodiments in which a nucleic acid payload can include (i) a signaling-enhanced EpoR transgene and (ii) an editing agent or system engineered to modify an endogenous nucleic acid sequence of a target cell, e.g., for a therapeutic purpose. The present disclosure further includes embodiments in which a nucleic acid payload can include an EpoR editing system engineered to modify an endogenous nucleic acid of a target cell that encodes EpoR and further engineered to modify another endogenous nucleic acid sequence of the target cell for a therapeutic purpose. In particular, a nucleic acid payload can include a “multiplexed” editing system in which the editing system is engineered to achieve editing of multiple targets using a single editing enzyme, optionally where one or more of the targets are therapeutic targets. A multiplexed editing system encoded by a nucleic acid payload can include a nucleic acid sequence that encodes an editing enzyme and two or more nucleic acid sequences that encode targeting agents (e.g., sgRNAs or pegRNAs) that direct the editing enzyme to edit two or more distinct targets.

Various components that can be included in and/or encoded by a nucleic acid payload are further disclosed herein. A nucleic acid payload can include any of one or more coding sequences that encode one or more expression products, one or more regulatory sequences operably linked to a coding sequence, one or more stuffer sequences, and the like. In various embodiments, the payload is engineered in order to achieve a desired result such as a therapeutic effect in a host cell or system, e.g., expression of a protein of therapeutic interest or of expression of a gene editing system, e.g., a CRISPR/Cas system, base editing system, or prime editing system to generate a sequence modification of therapeutic interest, e.g., to correct a nucleic acid lesion.

Nucleic acid payloads of the present disclosure can include a gene. A gene can include not only coding sequences but also regulatory regions such as promoters, enhancers, termination regions, locus control regions (LCRs), termination and polyadenylation signal elements, splicing signal elements, silencers, insulators, and the like. A gene can include introns and other DNA sequences spliced from an expressed mRNA transcript, along with variants resulting from alternative splice sites. Coding sequences can also include alternative synonymous codon usage as compared to a reference sequence, e.g., codon usage modified as compared to a reference in accordance with codon preference of a specific organism or target cell type.

A nucleic acid payload can include a single gene or multiple genes. A payload can include a single coding sequence or a plurality of coding sequences. A payload can include a single regulatory sequence or a plurality of regulatory sequences. A payload can include a plurality of coding sequences where the individual expression products of the coding sequences function together, e.g., as in the case of an editing enzyme and guide RNA of an editing system, or independently, e.g., as two separate proteins that do not directly or indirectly bind. As will be appreciated by those of skill in the art, a payload or payload component (e.g., a coding sequence and/or regulatory sequence) that is not naturally and/or endogenously encoded by a vector, host cell, and/or target cell can be referred to herein as heterologous. A payload expression product (e.g., an editing enzyme or other polypeptide or guide RNA encoded by a nucleic acid payload) that is not naturally and/or endogenously encoded and/or expressed by a vector, host cell, and/or target cell can be referred to herein as heterologous.

For the avoidance of doubt, the present disclosure includes variants of amino acid and nucleic acid sequences provided herein. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

III(A). Payload expression products

A nucleic acid payload of the present disclosure can include one or more sequences that encode and/or express any of a variety of expression products. Exemplary payload expression products include proteins, including without limitation replacement therapy proteins for treatment of diseases or conditions characterized by low expression or activity of a biologically active protein as compared to a reference level. Exemplary expression products include CRISPR/Cas, base editor, and prime editor systems (e.g., for one or more therapeutic purposes such as providing a nucleic acid sequence encoding signaling-enhanced EpoR and/or repairing a genetic lesion or abnormality and/or treating a disease, disorder, or condition). Exemplary expression products include antibodies, CARs, and TCRs. Exemplary expression products include small RNAs. In embodiments in which a nucleic acid sequence encodes one or more therapeutic proteins, a nucleic acid sequence encoding the therapeutic protein may be found in the art and/or readily derived from or generated based on the relevant amino acid sequence. In various embodiments, a coding sequence can be codon optimized for expression in mammalian cells (e.g., human cells). A nucleic acid sequence such as a nucleic acid payload, or a portion thereof that encodes one or more expression products or includes one or more genes, can include one or more restriction enzyme sites at the 5′ and/or 3′ ends as a means for isolating the nucleic acid sequence from a particular nucleic acid context and/or positioning the nucleic acid sequence within another nucleic acid context.

In various embodiments, integration of all or a portion of a nucleic acid payload into a host cell genome is not required in order for delivery to the host cell to produce an intended or target effect, e.g., in certain instances in which the intended or target effect includes editing of the host cell genome by a CRISPR, base editor, or prime editor system. In various embodiments, integration of all or a portion of a nucleic acid payload is required or preferred in order for delivery a nucleic acid payload to produce an intended or target effect, e.g., where expression of a payload-encoded expression product is desired in progeny cells of a transduced target cell. In various embodiments, a nucleic acid payload can include a nucleic acid sequence engineered for integration into a host cell genome (an “integrating fragment”), e.g., by recombination or transposition.

Particular examples of payload expression products include γ-globin, Factor VIII, YC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLREIB, SLC46A1, a FANC family gene (e.g., FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), soluble CD40, CTLA, Fas L, an antibody (e.g., that specifically binds CD4, CD5, CD7, CD52, IL1, IL2, IL6, TNF, P53, PTPN22, or DRB1*1501/DQB1*0602), an antibody to TCR specifically present on autoreactive T cells, IL4, IL10, IL12, IL13, ILIRa, sIL1RI, sIL1RII, STNFRI, sTNFRII, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and other payload expression products described herein.

A therapeutic payload expression product can be selected to provide a therapeutically effective response against diseases related to red blood cells and clotting. In particular embodiments, the disease is a hemoglobinopathy like thalassemia, or a sickle cell disease/trait. A payload expression product may be, for example, an expression product that induces or increases production of hemoglobin; induces or increases production of β-globin, γ-globin, or α-globin, or increases the availability of oxygen to cells in the body. A payload expression product can be, for example, HBB or CYB5R3. Exemplary effective treatments may, for example, increase blood cell counts, improve blood cell function, or increase oxygenation of cells in patients. In another particular embodiment, the disease is hemophilia. A payload expression product can be, for example, an expression product that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary payload expression products include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a nucleic acid payload encodes a globin gene, wherein the globin protein encoded by the globin gene is selected from a γ-globin, a β-globin, and/or an a-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or a-globin is a component of fetal and/or adult hemoglobin and is therefore useful to express in various methods and compositions disclosed herein, e.g., for treatment of a subject in need thereof.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or a-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1-4 relate to a-type globin sequences and references 4-12 relate to β-type globin sequences (including β and γ globin sequences), which sequences are hereby incorporated by reference: (1) GenBank Accession No. Z84721 (Mar. 19, 1997); (2) GenBank Accession No. NM_000517 (Oct. 31, 2000); (3) Hardison et al., J. Mol. Biol. (1991) 222 (2): 233-249; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993) or U01317.1; (6) Tagle et al., Genomics (1992) 13 (3): 741-760; (7) Grovsfeld et al., Cell (1987) 51 (6): 975-985; (8) Li et al., Blood (1999) 93 (7): 2208-2216; (9) Gorman et al., J. Biol. Chem. (2000) 275 (46): 35914-35919; (10) Slightom et al., Cell (1980) 21 (3): 627-638; (11) Fritsch et al., Cell (1980) 19 (4): 959-972; (12) Marotta et al., J. Biol. Chem. (1977) 252 (14): 5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS.; 92:6728-6732, 1995. In some embodiments, a globin gene encodes a G16D gamma globin variant.

An exemplary amino acid sequence of hemoglobin subunit β is provided, for example, at NCBI Accession No. P68871. An exemplary amino acid sequence for β-globin is provided, for example, at NCBI Accession No. NP 000509.

Nucleic acid payloads can also encode therapeutic molecules such as checkpoint inhibitor reagents, chimeric antigen receptors (e.g., chimeric antigen receptors specific to one or more cancer antigens), and/or T-cell receptors (e.g., T-cell receptors specific to one or more cancer antigens).

As another example, a payload expression product can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; or Fabry disease. A payload expression product can be, for example, an agent that induces production of an enzyme, or that otherwise causes degradation of mucopolysaccharides in lysosomes. Exemplary payload expression products can include IDUA or iduronidase, IDS, GNS, HGSNAT, SGSH, NAGLU, GUSB, GALNS, GLB1, ARSB, and HYAL1. Therapeutic nucleic acid payloads for lysosomal storage disorders may, for example, encode or induce the production of enzymes responsible for the degradation of various substances in lysosomes; reduce, eliminate, prevent, or delay the swelling in various organs, including the head (e.g., , Macrocephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a payload expression product can be can be selected to provide a therapeutically effective response against a hyperproliferative disease. In particular embodiments, the hyperproliferative disease is cancer. A payload expression product can be, for example, a tumor suppressor, an agent that induces apoptosis, an enzyme, a gene or polypeptide encoding an antibody, or polypeptide hormone. Exemplary payload expression products can include (in addition to those listed elsewhere herein) 101F6, 123F2 (RASSF1), 53BP2, abl, ABLI, ADP, aFGF, APC, ApoAI, ApoAIV, ApoE, ATM, BAI-1, BDNF, Beta* (BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, INGI, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

A payload expression product can be, for example, an agent useful for immune reconstitution, fighting infection (e.g., an antigen of an infectious agent, a receptor, a coreceptor, a receptor ligand, or a coreceptor ligand). Exemplary payload expression product can include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; a-dystroglycan; LDLR/α2MR/LRP; PVR; PRR1/HveC; and laminin receptor. As another example, a payload expression product can be selected to provide a therapeutically effective response against an infectious disease.

III(A)(1). Gene Editing Systems and Components for Modification of Endogenous Nucleic Acids Encoding EpoR and/or Other Expression Products

In various embodiments, a payload of the present disclosure encodes and/or expresses at least one component, or all components, of a gene editing system. Gene editing systems of the present disclosure include base editing systems, prime editing systems, CRISPR systems, zinc finger nucleases, and TALENs. Certain gene editing systems can include a plurality of components including a gene editing enzyme selected from a CRISPR-associated RNA-guided endonuclease, a base editing enzyme, and a prime editing enzyme, optionally in combination with at least one gRNA. Accordingly, gene editing systems of the present disclosure can include either (i) in the case of a CRISPR system, a CRISPR enzyme that is a CRISPR-associated RNA-guided endonuclease and at least one guide RNA (gRNA), (ii) in the case of a base editing system, a base editing enzyme and at least one gRNA, or (iii) in the case of a prime editing system and at least one prime editing gRNA. In certain embodiments, a gene editing system can include engineered zinc finger nucleases (ZFN). For instance, a ZFN is an artificial endonuclease that consists of a designed zinc finger protein (ZFP) fused to the cleavage domain of the FokI restriction enzyme. A ZFN may be redesigned to cleave new targets by developing ZFPs with new sequence specificities. For genome engineering, a ZFN is targeted to cleave a chosen genomic sequence. The cleavage event induced by the ZFN provokes cellular repair processes that in turn mediate efficient modification of the targeted locus. If the ZFN-induced cleavage event is resolved via non-homologous end joining, this can result in small deletions or insertions, effectively leading to gene knockout. If the break is resolved via a homology-based process in the presence of an investigator-provided donor, small changes or entire transgenes can be transferred, often without selection, into the chromosome, which can be referred to as ‘gene correction’ and ‘gene addition,’ respectively.

The present disclosure includes compositions including a nucleic acid that encodes an editing system disclosed herein (which nucleic acid can be referred to as an “editing payload”). A nucleic acid payload of the present disclosure can include one or more fragments each encoding one or more components of the editing system (and/or a fragment encoding the editing enzyme) operably linked with regulatory sequences such as a promoter. In various embodiments, one or more fragments of a nucleic acid payload that encode one or more components of an editing system can be referred to as an “EpoR editing payload.” A nucleic acid payload can further include a “therapeutic payload.” A therapeutic payload can refer to one or more fragments of a nucleic acid that encode one or more agents that cause, elicit, or contribute to a desired pharmacological and/or physiological effect (e.g., treatment of a disease, disorder, or condition) not achieved by modification of endogenous EpoR-encoding nucleic acids alone.

For avoidance of doubt, an editing payload of the present disclosure refers to any nucleic acid payload that encodes an editing system and can further include additional sequences including, for example, other payloads and/or functional sequences that do not perform or contribute to editing. Thus, to provide one example, an editing payload can refer to a viral vector genome that includes a nucleic acid payload that includes an EpoR editing payload, and optionally further includes a therapeutic payload.

In some embodiments a gene editing system (e.g., a CRISPR system, base editing system, or prime editing system) is engineered to modify a nucleic acid sequence that encodes y-globin, e.g., to increase expression of γ-globin. The main fetal form of hemoglobin, hemoglobin F (HbF) is formed by pairing of γ-globin polypeptide subunits with a-globin polypeptide subunits. Human fetal γ-globin genes (HBG1 and HBG2, two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34 (12): 927-940, 2018, which is incorporated herein by reference in its entirety and with respect to mutations that increase expression of γ-globin). Certain such mutations are found in the HBG1 promoter or HBG2 promoter.

In various embodiments, a gene editing system designed to increase expression of γ-globin includes an HBG1/2 promoter-targeted gRNA that is designed to increase expression of γ-globin by modification and/or inactivation of a BCL11A repressor protein binding site. In various embodiments, a gene editing system designed to increase expression of γ-globin includes a bcl11a-targeted gRNA that is designed to increase expression of γ-globin by modification and/or inactivation of the erythroid bcl11a enhancer to reduce BCL11A repressor protein expression in erythroid cells. In various embodiments, a gene editing system designed to increase expression of γ-globin includes a gRNA targeted to cause a loss of function mutation in the gene encoding BCL11A.

III(A)(1)(a). Base Editor Payload Expression Products for Modification of Endogenous Nucleic Acids Encoding EpoR and/or Other Expression Products

A payload expression product of the present disclosure can be a base editing system or one or more components thereof. In various embodiments, the present disclosure includes editing systems that utilize a deaminase (e.g., a base editing system) for editing of nucleic acid targets, including in various embodiments modification of an EpoR-encoding nucleic acid to produce a modified nucleic acid encoding signaling-enhanced EpoR. The present disclosure includes, among other things, base editing agents and systems, and nucleic acids encoding the same, e.g., where the nucleic acid is present in a nucleic acid payload. A base editing system can include a base editing enzyme and/or at least one gRNA as components thereof. A base editing system can utilize a deaminase (e.g., a base editing system) for editing of nucleic acid targets. In certain particular embodiments, a base editing agent and/or a base editing system of the present disclosure is present in a nucleic acid payload.

Deamination is the removal of an amine group from a molecule such as a nucleotide of a nucleic acid. Deamination of a nucleotide can cause changes in the sequence of a nucleic acid, and deaminases are useful in editing for at least that reason. Deamination of adenosine (A) yields inosine (I), which has the same base pairing preferences as a guanosine in DNA and is thus recognized by cell replication machinery as guanosine, resulting in an A-T to G-C transition. Deamination of cytosine (C) yields uridine (U), which is recognized by cell replication machinery as thymine, resulting in a C-G to T-A transition. Collectively, cytosine and adenosine deamination can be used to cause transitions from A to G, T to C, C to T, or G to A. Other deaminase activities are also known. For example, deamination of 5-methylcytosine yields thymine and deamination of guanosine yields xanthine, though xanthine, like guanosine, pairs with cytosine. Deaminases that deaminate cytosine can be referred to as cytosine deaminases. Deaminases that deaminate adenosine can be referred to as adenosine deaminases.

In particular embodiments, a base editing enzyme includes a cytidine deaminase domain or an adenine deaminase domain. Certain embodiments utilize a cytidine deaminase domain as the nucleobase deaminase enzyme. Particular embodiments utilize an adenine deaminase domain as the nucleobase deaminase enzyme.

Examples of cytosine deaminase enzymes (CBEs) include APOBEC1, APOBEC3A, APOBEC3G, CDA1, and AID. APOBEC1 particularly accepts single-stranded (ss) DNA as a substrate but is incapable of acting on double-stranded (ds) DNA.

For adenosine base editors (ABEs), exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, an ABE can include one or more, or all, of three components including a wild-type E. coli tRNA-specific adenosine deaminase (TadA) monomer, which can play a structural role during base editing, a TadA* mutant TadA monomer that catalyzes deoxyadenosine deamination, and/or a Cas nickase such as Cas9 (D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers has a sequence according to (SGGS)₂-XTEN-(SGGS)₂ (SEQ ID NO: 214) or a sequence otherwise known to those of skill in the art.

In various embodiments, an editing system includes a deaminase associated with a DNA binding domain such as a catalytically impaired nuclease domain. In various embodiments, the DNA binding domain can localize the deaminase to a target nucleic acid in which one or more nucleotides are deaminated by the deaminase. Catalytically impaired nuclease domains are polypeptide domains that have amino acid sequences engineered from reference nuclease domain sequences but that have a reduced ability to cause double-strand breaks (DSBs) as compared to the reference (e.g., a wild type and/or fully functional nuclease) or have no ability to cause double-strand breaks. As referred to herein, a nickase refers to a catalytically impaired nuclease domain that, upon contact with a double-stranded nucleic acid substrate, cleaves one strand (e.g., a target strand) of the double-stranded nucleic acid but not both strands of the double-stranded nucleic acid. In various embodiments, a nickase, upon contact with a double-stranded nucleic acid substrate, cleaves one strand of the double-stranded nucleic acid but not both strands of the double-stranded nucleic acid in at least 70% of contacted double-stranded nucleic acid substrates (e.g., at least 70%, 75%, 80%, 85%, 90%, 91%, 92%, 93%, 94%, 95%, 96%, 97%, 98%, 99%, or 100% of double-stranded nucleic acid substrates).

Base editing systems are exemplary of editing systems that include deaminase enzymes. A base editing enzyme includes a deaminase enzyme fused to a DNA binding domain that is a catalytically impaired nuclease domain (e.g., a nickase, e.g., a nickase that nicks a single strand, e.g., a non-edited strand). DNA binding domains of base editing enzymes can be RNA guided DNA binding domains, in that an RNA guide can direct the DNA binding domain to a target nucleic acid sequence. Catalytically impaired nuclease domains of a base editing enzyme can bind nucleic acids and can localize the deaminase enzyme to a target nucleic acid.

Any nuclease of the CRISPR system can be engineered to produce a catalytically impaired nuclease domain (e.g., a nickase) and used within a base editing enzyme or system. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

In various embodiments, a catalytically impaired nuclease domain generates a single-stranded nick in the non-deaminated DNA strand, inducing cells to repair the non-deaminated strand using the deaminated strand as a template. To provide one example, nCas9 can create a nick in target DNA by cutting a single strand, reducing the likelihood of detrimental indel formation as compared to methods that require a double-strand break.

Particular embodiments utilize a nuclease-inactive Cas9 (dCas9) as the catalytically disabled nuclease. However, any nuclease of the CRISPR system (many of which are described above) can be disabled and used within a base editing system. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver (2016 Nature 529:490-495) and Slaymaker (2015 Science 351:84-88).

Other DNA binding nucleases can also be used in a base editing enzyme. For example, base-editing systems can utilize zinc finger nucleases (ZFNs) (see, e.g., Urnov 2010 Nat Rev Genet. 11 (9): 636-46) and transcription activator like effector nucleases (TALENs) (see, e.g., Joung 2013 Nat Rev Mol Cell Biol. 14 (1): 49-55). For additional information regarding DNA-binding nucleases, see, e.g., US 2018/0312825.

In various embodiments, a base editing enzyme includes a DNA glycosylase inhibitor. A DNA glycosylase inhibitor can override natural DNA repair mechanisms that might otherwise repair the intended base editing. A DNA glycosylase inhibitor can be a uracil DNA glycosylase inhibitor protein (UGI). One exemplary UGI is described in Wang (1991 Gene 99:31-37). In particular embodiments, a base editing enzyme can include one or more DNA glycosylase inhibitor domains (e.g., UGI domains). In various embodiments, base editing enzymes that include more than one DNA glycosylase inhibitor domain (e.g., UGI domain) can generate fewer indels and/or deaminate target nucleic acids more efficiently than base editing enzymes that includes one DNA glycosylase inhibitor domain (e.g., UGI domain) and/or no DNA glycosylase inhibitor domains (e.g., UGI domains). For example, in particular embodiments, dCas9 or a Cas9 nickase can be fused to a cytidine deaminase domain and the dCas9 or Cas9 nickase can be fused to one or more UGI domains.

In particular embodiments, a deaminase domain is associated with the N-terminus of a catalytically disabled nuclease. In particular embodiments, a deaminase domain is associated with the N-terminus of a catalytically disabled nuclease. In certain embodiments, one or more glycosylase inhibitors (e.g., UGI domain) can be associated with the C-terminus of a catalytically disabled nuclease.

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester), amino acid linkers, carbon-nitrogen bond amide linkers, cyclic linkers, acyclic linkers, substituted linkers, unsubstituted linkers, branched linkers, unbranched aliphatic or heteroaliphatic linkers, aminoalkanoic acid linkers (e.g., monomeric, dimeric, or polymeric aminoalkanoic acid linkers), aminoalkanoic acid linkers (e.g., glycine, ethanoic acid, alanine, β-alanine, 3-aminopropanoic acid, 4-aminobutanoic acid, 5-pentanoic acid linkers), aminohexanoic acid (Ahx) linkers (e.g., monomeric, dimeric, or polymeric Ahx linkers), carbocyclic moiety (e.g., cyclopentane, cyclohexane) linkers, aryl or heteroaryl moiety linkers, and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Various base editing enzymes are known in the art. Examples of base editing enzymes include BE1 (APOBEC1-16 amino acid (aa) linker-Sp dCas9 (D10A, H840A) (see, e.g., Komor 2016 Nature 533:420-424)), BE2 (APOBEC1-16aa linker-Sp dCas9 (D10A, H840A)-4aa linker-UGI (see, e.g., Komor 2016 Nature 533:420-424)), BE3 (APOBEC1-16aa linker-SpnCas9 (D10A)-4aa linker-UGI (see, e.g., Komor 2016 Nature 533:420-424)), HF-BE2 (rAPOBEC1-HF2 nCas9-UGI), HF-BE3 (APOBEC1-16aa linker-HF nCas9 (D10A)-4aa linker-UGI (see, e.g., Rees 2017 Nat. Commun. 8:15790)), BE4 (rAPOBEC1-Sp nCas9-UGI-UGI), BE4max (APOBEC1-32aa linker-Sp nCas9 (D10A)-9aa linker-UGI-9aa linker-UGI (see, e.g., Koblan 2018 Nat. Biotechnol 36 (9): 843-846 and/or Komor 2017 Sci. Adv. 3 (8): eaao4774)), BE4-GAM (Gam-16aa linker-APOBEC1-32aa linker-Sp nCas9 (D10A)-9aa linker-UGI-9aa linker-UGI (see, e.g., Komor 2017 Sci. Adv. 3 (8): eaao4774)), YE1-BE3 (APOBEC1 (W90Y, R126E)-16aa linker-Sp nCas9 (D10A)-4aa linker-UGI (see, e.g., Kim 2017 Nat. Biotechnol. 35:475-480)), EE-BE3 (APOBEC1 (R126E, R132E)-16aa linker-Sp nCas9 (D10A)-4aa linker-UGI (see, e.g., Kim 2017 Nat. Biotechnol. 35:475-480)), YE2-BE3 (APOBEC1 (W90Y, R132E)-16aa linker-Sp nCas9 (D10A)-4aa linker-UGI (see, e.g., Kim 2017 Nat. Biotechnol. 35:475-480)), YEE-BE3 (APOBEC1 (W90Y, R126E, R132E)-16aa linker-Sp nCas9 (D10A)-4aa linker-UGI (see, e.g., Kim 2017 Nat. Biotechnol. 35:475-480)), VQR-BE3 (APOBEC1-16aa linker-Sp VQR nCas9 (D10A)-4aa linker-UGI (see, e.g., Kim 2017 Nat. Biotechnol. 35:475-480)), EQR-BE3 (rAPOBEC1-EQR SpnCas9-UGI), VRER-BE3 (APOBEC1-16aa linker-Sp VRER nCas9 (D10A)-4aa linker-UGI (see, e.g., Kim 2017 Nat. Biotechnol. 35:475-480)), Sa-BE3 (APOBEC1-16aa linker-Sa nCas9 (D10A)-4aa linker-UGI (see, e.g., Kim 2017 Nat. Biotechnol. 35:475-480)), SA-BE4 (APOBEC1-32aa linker-Sa nCas9 (D10A)-9aa linker-UGI-9aa linker-UGI (see, e.g., Komor 2017 Sci. Adv. 3 (8): eaao4774)), SaBE4-Gam (Gam-16aa linker-APOBEC1-32aa linker-Sa nCas9 (D10A)-9aa linker-UGI-9aa linker-UGI (see, e.g., Komor 2017 Sci. Adv. 3 (8): eaao4774)), SaKKH-BE3 (APOBEC1-16aa linker-Sa KKH nCas9 (D10A)-4aa linker-UGI (see, e.g., Kim 2017 Nat. Biotechnol. 35:475-480)), FNLS-BE3 (rAPOBEC1-Sp nCas9-UGI), RA-BE3 (rAPOBEC1 (RA)-Sp nCas9-UGI), Cas12a-BE (APOBEC1-16aa linker-dCas12a-14aa linker-UGI (see, e.g., Li 2018 Nat. Biotechnol. 36:324-327)), Target-AID (Sp nCas9 (D10A)-100aa linker-CDA1-9aa linker-UGI (see, e.g., Nishida 2016 Science 353 (6305): aaf8729)), Target-AID-NG (Sp nCas9 (D10A)-NG-100aa linker-CDA1-9aa linker-UGI (see, e.g., Nishimasu 2018 Science 361 (6408): 1259-1262)), xBE3 (APOBEC1-16aa linker-xCas9 (D10A)-4aa linker-UGI (see, e.g., Hu 2018 Nature 556:57-63)), eA3A-BE3 (APOBEC3A (N37G)-16aa linker-Sp nCas9 (D10A)-4aa linker-UGI (see, e.g., Gehrke 2018 Nat. Biotechnol. 36 (10): 977-982)), A3A-BE3 (hAPOBEC3A-16aa linker-Sp nCas9 (D10A)-4aa linker-UGI (see, e.g., Wang 2018 Nat. Biotechnol. 36:946-949)), eA3A-HF1-BE3-2×UGI (APOBEC3A-HF1 Sp nCas9-UGI-UGI), eA3A-HypaBE3-2×UGI (APOBEC3A-Hypa Sp nCas9-UGI-UGI), hA3A-BE3 (hAPOBEC3A-Sp nCas9-UGI), hA3B-BE3 (hAPOBEC3B-Sp nCas9-UGI), hA3G-BE3 (hAPOBEC3G-Sp nCas9-UGI), hAID-BE3 (hAPOBEC3A-Sp nCas9-UGI), SaCas9-BE3 (rAPOBEC1-SanCas9-UGI), xCas9-BE3 (rAPOBEC1-xnCas9-UGI), ScCas9-BE3 (rAPOBEC1-ScnCas9-UGI), SniperCas9-BE3 (rAPOBEC1-SnipernCas9-UGI), iSpyMac-BE3 (rAPOBEC1-iSpyMacnCas9-UGI), CRISPR-X (Sp dCas9-MS2-hAID), TAM (Sp dCas9-hAID (P182X)), AncBE4-Max (rAPOBEC1-Sp nCas9-UGI-UGI), ABE7.8/9/10 (ecTadA-ecTadA *-Sp nCas9), xCas9-ABE7.10 (ecTadA-ecTadA *-nxCas9), VQR-ABE (ecTadA-ecTadA *-Sp VQR nCas9), Sa (KKH)-ABE ecTadA-ecTadA *-Sa KKH nCas9), ABEmax (ecTadA-ecTadA *-Sp nCas9), ABE7.10max (ecTadA-ecTadA *-SpnCas9), ABE8e) ecTadA-ecTadA *-SpnCas9), PE1 (dSpCas9-MMLV-RT), PE2 (dSpCas9-MMLV-RT), PE3 (nSpCas9-MMLV-RT), and BE-PLUS (10X GCN4-Sp nCas9 (D10A)/ScFv-rAPOBEC1-UGI (see, e.g., Jiang 2018 Cell Res. 28 (8): 855-861)). For additional examples of BE complexes, including adenine deaminase base editors, see, e.g., Rees 2018 Nat. Rev Genet. 19 (12): 770-788 and/or Kantor 2020 Int. J. Mol. Sci. 21 (17): 6240.

Various base editors are “dual base editors” that can edit both adenine and cytosine. Dual base editor enzymes can be fusion polypeptides that include a cytosine deaminase domain and an adenine deaminase domain. For instance, a dual base editor known as Target-ACEmax includes a codon-optimized fusion of the cytosine deaminase PmCDA1, the adenosine deaminase TadA, and a Cas9 nickase (Target-ACEmax) (see, e.g., Sakata 2020 Nature Biotechnology, 38 (7), 865-869). Other exemplary dual base editors include SPACE (synchronous programmable adenine and cytosine editor). The SPACE editing enzyme is a fusion polypeptide that includes both miniABEmax-V82G and Target-AID editing domains together with a Cas9 (SpCas9-D10A) nickase domain (see, e.g., Grünewald 2020 Nat. Biotechnol. 38:861-864). A dual base editor known as A&C-BEmax includes a fusion of both cytidine and adenosine deaminase domains with a Cas9 nickase domain (see, e.g., Zhang 2020 Nat. Biotechnol. 38:856-860).

A base editing system can include a guide RNA (gRNA) that includes at least a fragment that base pairs with a complementary target nucleic acid (e.g., at least 80% identity between the fragment and the complement of the target nucleic acid, e.g., 80%, 85%, 90%, 91%, 92%, 93%, 94%, 95%, 96%, 97%, 98%, 99%, or 100% identity), wherein the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length), e.g., where the target sequence is upstream of an appropriate PAM site. In various embodiments, a fragment of a gRNA that is complementary to a target nucleic acid sequence is positioned at the 5′ end of a gRNA or is 5′ relative to one or more other fragments of the gRNA. In various embodiments, a gRNA includes a sequence that forms a stemloop structure and binds with and/or recruits the catalytically impaired nuclease domain of a base editing enzyme. A gRNA that includes both a fragment that base pairs with a complementary target nucleic acid sequence and a fragment that forms a stemloop structure and binds with and/or recruits the catalytically impaired nuclease domain of a base editing enzyme can be referred to as a single guide RNA (sgRNA). The fragments of sgRNA can be associated via a linker fragment. In some embodiments, a guide RNA includes a nucleic acid sequence corresponding to TGTCCATGGACGCAAGAGCT (SEQ ID NO: 657) or a nucleic acid sequence having at least 80%, 85%, 90%, or 95% identity thereto. In some embodiments, a guide RNA includes a nucleic acid sequence corresponding to GTGTCCATGGACGCAAGAGC (SEQ ID NO: 658) or a nucleic acid sequence having at least 80%, 85%, 90%, or 95% identity thereto.

A guide RNA (e.g., an sgRNA) is thought to randomly interrogate nucleic acids until it encounters a nucleic acid that is sufficiently complementary to the 5′ fragment. Upon binding of a gRNA to a DNA nucleic acid target present in double-stranded DNA, base pairing between the gRNA and target nucleic acid strand causes displacement of a small segment of single-stranded DNA. In various embodiments, the gRNA recruits the catalytically impaired nuclease domain. Nucleotides of the displaced single-stranded DNA can be modified by the deaminase enzyme. The resultant base pair can then be repaired by cellular mismatch repair machinery to a new base pair, or alternatively in some instances reverted by base excision repair mediated by uracil glycosylase. In various embodiments, a glycosylase inhibitor (e.g., UGI) reduces the occurrence of reversion. In various embodiments, a gRNA recruits a base editor to modify an endogenous EpoR-encoding nucleic acid to produce a nucleic acid encoding a signaling-enhanced EpoR polypeptide. In various embodiments, a gRNA recruits a base editor to modify an endogenous EpoR-encoding nucleic acid to produce a modified nucleic acid according to Table 1.

The present disclosure includes base editing enzymes and systems engineered to increase the editing window of base editing. For example, the present disclosure includes circularly permuted base editors, described for example in Huang 2020 Nature Biotechnology, 37 (6), 626-631, which is incorporated herein with respect to base editing enzymes, base editing systems, and editing windows thereof. Circularly permuted base editing enzymes and systems can be characterized by an increased range of target bases that can be modified within the protospacer up to and including, for example, at least 5, 6, 7, 8, or 9 nucleotides. For example, certain base editing systems including Cas9 variants, including cytosine and four adenine base editing enzymes, can deaminated nucleotides in a window expanded from about 4-5 nucleotides to up about 8-9 nucleotides, optionally with reduced byproduct formation.

Base editing enzymes and systems can also target and/or modify RNA molecules. One advantage of using RNA editing systems is that there is no permanent change in the genome. RNA base editors achieve analogous changes using components that base modify RNA. For example, adenosine deaminase can modify transcribed mRNA, replacing adenosine with inosine at a target site. In mammals, the most prevalent post-transcription RNA editing case is catalyzed by the adenosine deaminase enzymes (ADARs). ADAR proteins are a highly conserved family of proteins that include a single deaminase domain (DD) and one or more double-stranded RNA (dsRNA)-binding domains ADARs (e.g., ADAR 1 or ADAR2) bind to dsRNA and catalyzes adenosine to inosine (A-to-I), which is read as guanosine by cellular translational machinery. ADAR1 and ADAR2 domains have been demonstrated to achieve RNA editing, e.g., in HSCs (see, e.g., Harter 2009 Nat. Immunol. 10 (1): 109-115). A number of catalytically inactive Cas proteins have also been used to target RNA molecules, including Cas9, Cas13a, Cas13b, and Cas13d.

REPAIR (RNA editing for programmable adenosine to inosine replacement) is an RNA base editing system that includes catalytically inactive Cas13 protein and the deaminase activity of ADAR2. Cas13 generally includes two HEPN (higher eukaryotes and prokaryotes nucleotide-binding) domains, which contribute to RNA-targeted nucleolytic activity. Mutations of HEPNs abolish RNA cleavage activity while maintaining RNA targeting activity, which has been used to create an RNA base editing enzyme (e.g., REPAIR) (see, e.g., Cox 2017 Science 358:1019-1027). dCas13-ADAR2_{DD} includes catalytically inactive dCas13 variant with RNA deaminase ADAR2 (E488Q), and can execute RNA editing for programmable A-to-I (G) replacement. RNA Editing for Specific C-to-U Exchange (RESCUE) was later developed (see, e.g., Abudayyeh 2019 Science 365:382-386). gRNAs for mRNA editing can include, e.g., a fragment complementary to a target RNA and an ADAR-recruiting fragment, such that site-directed RNA editing is achieved by recruiting ADAR to a complementary target nucleic acid. RNA-guided RNA-targeting CRISPR nuclease C2C2 (later named as Cas13a) from Leptotrichia shahii was illustrated (Abudayyeh 2016 Science 353: aaf5573).

Other examples of RNA editing systems that include ADARs can include removing the endogenous RNA-targeting domains (dsRBMS) from human adenosine deaminase and replacing them with an antisense RNA oligonucleotide to produce a recombinant enzyme that can be directed to edit a selected RNA target. In particular embodiments, an ADAR2 deaminase domain is fused with an RNA-binding protein, and the sequence bound by the RNA-binding protein is associated with an antisense RNA guide oligonucleotide. In various embodiments, the RNA-binding protein is derived from λ-phage N protein-boxB RNA interaction, which normally regulates antitermination during transcription of λ-phage mRNAs. λN peptide mediates binding of the N protein, is only 22 amino acids long, and the boxB RNA hairpin that it recognizes is only 17 nucleotides long and they can bind with nanomolar affinity. Thus, in various embodiments, λN peptide can be fused to the deaminase domain of human ADAR2 (λN-DD). In various embodiments, a mutant ADAR2_{DD} (E488Q) can be used as the deaminase domain. In various embodiments, an editing enzyme can include an ADAR deaminase domain and 2 or more λN domains (e.g., 2, 3, 4, 5, or 6 λN domains). Examples of such editing enzymes and systems are described, e.g., in Montiel-Gonzalez 2013 PNAS 110 (45): 18285-18290 and Montiel-Gonzalez 2016 Nuc. Acids. Res. 44 (2): e157, each of which is incorporated herein by reference with respect to editing systems.

Other examples of editing systems that include ADARs can include leveraging endogenous ADAR for programmable editing of RNA (LEAPER) editing system that employs short engineered ADAR-recruiting RNAs (arRNAs) to recruit native ADAR1 or ADAR2 deaminase enzymes to change a specific adenosine to inosine. For example, in certain particular embodiments, an ADAR protein or its catalytic domain can be fused with a λN peptide. In certain embodiments, an ADAR protein or its catalytic domain can be fused with a λN peptide and a SNAP-tag or a Cas protein (e.g., dCas13b). A gRNA can recruit the editing enzyme to the specific site. Further description of LEAPER editing systems can be found in Qu 2019 Nat. Biotech. 1059-1069, which is incorporated herein by reference with respect to LEAPER editing systems and

Base editing systems can cause point mutations without producing double-strand breaks. Base editing systems can cause point mutations without producing undesired insertions and deletions (indels). For example, a base editing system can cause indels in less than 10%, 9%, 8%, 7%, 6%, 5.5%, 5%, 4.5%, 4%, 3.5%, 3%, 2.5%, 2%, 1.5%, 1%, 0.5%, or 0.1% of edited cells or editing events.

Those of skill in the art will appreciate that a base editing gRNA (e.g., sgRNA) or other targeting elements to generate a selected nucleic acid sequence modification in a target nucleic acid can be readily designed and implemented, e.g., based on available sequence information.

Base editing systems do not require double-stranded DNA breaks. Base editing systems do not require a donor fragment or template. Base editing systems provide precise control of the site at which the editing system modifies a target nucleic acid. Base editing systems can be multiplexed to achieve editing of multiple targets using a single editing enzyme, optionally including therapeutic targets. The present disclosure includes base editing systems that include a plurality of sgRNAs (e.g., two or more, e.g., two, three, four, or five) sgRNAs. For example, in various embodiments a first sgRNA modifies (e.g., is engineered to modify or physically modifies) an EpoR-encoding nucleic acid sequence and a second sgRNA modifies a therapeutic target, e.g., a gene comprising a genetic lesion associated with a disease, disorder, or condition. In various embodiments, a first sgRNA modifies one or more nucleotide positions at a first locus within an EpoR-encoding nucleic acid and a second sgRNA modifies one or more different nucleotide positions at a second different locus within an EpoR-encoding nucleic acid, optionally where at least a third sgRNA can modify a therapeutic target.

III(A)(1)(b). Prime editor payload expression products for modification of endogenous nucleic acids encoding EpoR and/or other expression products

A payload expression product of the present disclosure can be a prime editing system or one or more components thereof. In various embodiments, the present disclosure includes editing systems that utilize a reverse transcriptase (e.g., a prime editing system) for editing of nucleic acid targets, including in various embodiments modification of an EpoR-encoding nucleic acid to produce a modified nucleic acid encoding signaling-enhanced EpoR. The present disclosure includes, among other things, prime editing agents and systems, and nucleic acids encoding the same, e.g., where the nucleic acid is present in a nucleic acid payload. A prime editing system can include a prime editing enzyme and/or at least one pegRNA as components thereof. Prime editing can introduce all possible types of point mutations, small insertions, and small deletions in a precise and targeted manner. A prime editing enzyme includes a reverse transcriptase fused to a DNA binding domain that is a catalytically impaired nuclease domain (e.g., a nickase, e.g., a nickase that nicks a single strand, e.g., a non-edited strand). A reverse transcriptase is an enzyme that can synthesize a DNA molecule from an RNA template. A reverse transcriptase generally produces a DNA molecule that is complementary to the RNA template.

In particular embodiments, an editing enzyme includes an AMV reverse transcriptase, MLV reverse transcriptase, HIV-1 reverse transcriptase, or bacterial reverse transcriptase. Certain embodiments utilize an MLV reverse transcriptase domain. Reverse transcriptases of the present disclosure can have wild type amino acid sequences or engineered amino acid sequences.

Examples of reverse transcriptase enzymes include AMV reverse transcriptases (e.g., wild type AMV reverse transcriptase (RNase H plus activity), eAMV™ (engineered; RNase Hplus activity) or THermoScript™ (engineered; reduce RNAase H activity)), MLV reverse transcriptases (e.g., wild type M-MLV reverse transcriptase, GoScript™, or MultiScribe™ (RNase H plus activity), AccuScript Hi-Fi (engineered, RNase H minus (3′-5′ exonuclease activity), Affinity Script (engineered; E69K/E302R/W313F/L435G/N454K; unspecified RNase H activity), ArrayScript™ (engineered; unspecified RNase H activity), BioScript™ (engineered; reduced RNase H activity), CycleScript™ (engineered), EnzScript™ (engineered; RNase H minus), EpiScript™ (engineered; RNase H minus), Expand™ reverse transcriptase (engineered; RNase H reduced), FIREScript (engineered; RNase H plus), GrandScript (engineered; RNase H plus), iScript™ (engineered; RNase H plus), Maxima™ RT (engineered; RNase H plus and minus), MonsterScript™ (engineered; RNase H minus), PrimeScript™ (engineered; RNase H minus), PrimeScript™ II (engineered; RNase H minus), PrimeScript™ III(engineered; RNase H minus), PrimeScript™ IV (engineered; RNase H minus), ProtoScript® (Engineered; RNase H plus), ProtoScript® II (engineered; RNase H reduced), qScript (engineered; RNase H plus), RevertAid™ (engineered; RNase H plus and minus), ReverTra Ace® (engineered; RNase H minus), RevertUp II™ (engineered; RNase H minus), Rocketscript™ (engineered; RNase H plus and minus), Script (engineered; RNase H minus), SMART® (engineered), SMARTScribe™ (engineered; unspecified RNase H activity), SuperScript™ II (engineered; 524G/D583N/E562Q; RNase H reduced), SuperScript™ III (engineered; 204R/V223H/T306K/F309N/D524G/D583N/E562Q; RNase H reduced), SuperScript™ IV (engineered; RNase H reduced), or Transcriptor reverse transcriptase (engineered; RNase H plus)), an HIV-1 reverse transcriptase (e.g., HIV-1 RT (wild type of group M subtype B; RNase H plus), Biotools high retrotranscriptase (engineered group O variant (K65R/V75I); RNase H plus), or Sunscript® (engineered group O variants with changes K358R/A359G/S360A; RNase H plus and minus)), a bacterial group II intron reverse transcriptase (e.g., Marathon RT (wild type (Eubacterium rectale); lacks RNase H domain) or TGIRT®-III RT (wild type (Geobacillus stearothermophilus); lacks RNase H domain), a bacterial DNA polymerase (e.g., BcaBEST polymerase (engineered (Bacillus caldotenax DNA polymerase without 5′-3′ and 3′-5′ exonuclease activity); lacks RNase H domain), Bst 3.0 DNA polymerase (G. stearothermophilus DNA polymerase I, large fragment; lacks 5′-3′ and 3′-5′ exonuclease activity; lacks RNase H domain), RapiDxFire™ reverse transcriptase (lacks RNase H domain), Volcano2G DNA polymerase (engineered Thermus aquaticus DNA polymerase; lacks RNase H domain), or Volcano3G DNA polymerase (engineered T. aquaticus DNA polymerase; lacks RNase H domain)), SOLIScript (engineered; RNase H reduced), Omniscript® (heterodimeric RT; RNase H plus), and SensiScript® (heterodimeric RT; RNase H plus).

In various embodiments, a reverse transcriptase is a retrovirus reverse transcriptase. In various embodiments, a reverse transcriptase is a murine leukemia virus (MLV) reverse transcriptase (RT) (e.g., an engineered MLV RT). In various embodiments, a reverse transcriptase is a bacterial group II intron RT.

In various embodiments, a prime editing enzyme or system includes a reverse transcriptase associated with a DNA binding domain such as a catalytically impaired nuclease domain. In various embodiments, the DNA binding domain can localize the reverse transcriptase to a target nucleic acid in which one or more nucleotides are substituted, inserted, and/or deleted.

DNA binding domains of prime editing enzymes can be RNA guided DNA binding domains, in that an RNA guide can direct the DNA binding domain to a target nucleic acid sequence. Catalytically impaired nuclease domains of a prime editing enzyme can bind nucleic acids and can localize the reverse transcriptase enzyme to a target nucleic acid in which one or more nucleotides are substituted, inserted, and/or deleted by the prime editing system.

Any nuclease of the CRISPR system can be engineered to produce a catalytically impaired nuclease domain (e.g., a nickase) and used within a prime editing enzyme or system. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, CasY, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

Other DNA binding nucleases can also be used in a prime editing enzyme. For example, prime editing systems can utilize zinc finger nucleases (ZFNs) (see, e.g., Urnov 2010 Nat Rev Genet. 11 (9): 636-46) and transcription activator like effector nucleases (TALENs) (see, e.g., Joung 2013 Nat Rev Mol Cell Biol. 14 (1): 49-55). For additional information regarding DNA-binding nucleases, see, e.g., US 2018/0312825.

In various embodiments, a prime editing system includes a prime editing gRNA (pegRNA) that specifies a target nucleic acid sequence and also specifies the sequence modification that the prime editing system introduces. The pegRNA includes a sequence complimentary to the target nucleic acid and recruits the prime editing enzyme to the target nucleic acid. A pegRNA includes, from 5′ to 3′: (a) a fragment that base pairs with a complementary target nucleic acid sequence (e.g., at least 80% identity between the fragment and the complement of the target nucleic acid, e.g., 80%, 85%, 90%, 91%, 92%, 93%, 94%, 95%, 96%, 97%, 98%, 99%, or 100% identity) (sometimes referred to as a “spacer”), wherein the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length); (b) a sequence that forms a stemloop structure and binds with and/or recruits the catalytically impaired nuclease domain of a prime editing enzyme; (c) a fragment that includes a sequence that includes one or more modifications (e.g., one or more substitutions, insertions, and/or deletions) relative to the target nucleic acid sequence (sometimes referred to as a “template sequence”), and is complementary (excepting modifications) to the same target nucleic acid strand as (d); and (d) a fragment that includes a sequence complimentary to a target sequence (sometimes referred to as a “binding region” or “primer binding site” (PBS)), e.g., where the target sequence is upstream of an appropriate PAM site. In various embodiments, a PBS can be 5 to 20 nucleotides, e.g., 8 to 15 nucleotides in length. In various embodiments, a template sequence can be 10 to 20 nucleotides in length, or longer. Because pegRNAs include components characteristic of sgRNAs, they are sometimes described as extended sgRNAs. Any two fragments of a pegRNA can be, independently, associated directly or via a linker fragment.

A catalytically impaired nuclease domain of a prime editing enzyme can nick a target nucleic acid that includes an appropriate PAM to expose a 3′ flap and a 5′ flap. After nicking of the target nucleic acid, the released 3′ flap can hybridize to the PBS of the pegRNA, priming reverse transcription of the template fragment of the pegRNA that includes a modification of the target sequence, directly introducing the modification into the target nucleic acid to the 3′ flap. The product of reverse transcription, an edited 3′ flap that is “redundant” with the 5′ flap sequence produced by the nick (which includes the original, unedited sequence of the target nucleic acid), can then compete with the original and redundant 5′ flap sequence for reincorporation into the DNA duplex. Although the perfectly complimentary 5′ would likely be thermodynamically favored for hybridization to the non-edited strand, the 5′ flap is preferentially degraded by cellular endonucleases that are ubiquitous during lagging-strand DNA synthesis. After 5′ flap excision and ligation of the edited strand, permanent installation of the edit occurs through DNA repair of the non-edited that relies on the edited strand as a template. DNA repair of the non-edited strand can be promoted by contact with a secondary sgRNA that directs nicking of the non-edited strand. This additional nick stimulates re-synthesis of the non-edited strand using the edited strand as a template, resulting in a fully edited duplex. Prime editing systems can introduce any of one or more of the 12 types of point mutations (all possible nucleotide transitions and transversions), as well as insertions and/or deletions.

In various embodiments, a prime editing system is engineered to disrupt a PAM site of a target nucleic acid. Disruption of a PAM site of a target nucleic acid can reduce the probability of repeated editing of the particular target nucleic acid. In various embodiments, disruption of a PAM site in edited target nucleic acids can increase the efficiency of prime editing and/or gene therapy that includes prime editing.

Exemplary prime editing systems include PE1, PE2, and PE3. Each of these prime editing enzymes include a mutant Streptococcus pyogenes Cas9 nickase domain (H840A mutant) and a Moloney murine leukemia virus (M-MLV) reverse transcriptase (e.g., engineered to include D200N/T306K/W313F/T330P/L603W). PE1 includes a pegRNA and a prime editing enzyme that includes a Cas9 H840A nickase and wild type MLV RT. The Cas9 nickase acts only on the strand to be edited by the RT. PE2 includes pegRNA and a prime editing enzyme that includes a Cas9 H840A nickase and engineered MLV RT (D200N/T306K/W313F/T330P/L603W) demonstrated to improve editing efficiency. PE3 includes the same prime editing enzyme as PE2 (as well as a pegRNA) but further includes an sgRNA that targets the non-edited strand for nicking 14-116 nucleotides away from the site of the pegRNA-induced nick (PE3), where cellular mismatch repair pathways can fix the information introduced in the edited strand. Compared with PE2, the PE3b strategy demonstrate increased editing efficiency and lower levels of indel formation. A variant of the PE3 system called PE3b uses a nicking sgRNA that targets only the edited sequence, resulting in decreased levels of indel products by preventing nicking of the non-edited DNA strand until the other strand has been converted to the edited sequence.

Those of skill in the art will appreciate that a pegRNA or other targeting elements to generate a selected nucleic acid sequence modification in a target nucleic acid can be readily designed and implemented, e.g., based on available sequence information. Various tools for designing pegRNAs are available. For example, pegFinder is a web-based tool for pegRNA design (see, e.g., Chow 2020 Nat. Biomed. Eng. doi: 10.1038/s41551-020-00622-8). Another example of a web-based tool for pegRNA design is PrimeDesign (see, e.g., Hsu 2020 bioRxiv doi: 10.1101/2020.05.04.077750).

Prime editing systems do not require double-stranded DNA breaks. Prime editing systems provide precise control of the site at which the editing system modifies a target nucleic acid. Prime editing systems can be multiplexed to achieve editing of multiple targets using a single editing enzyme, optionally including therapeutic targets. The present disclosure includes that a prime editing system can include a plurality of pegRNAs (e.g., two or more, e.g., two, three, four, or five pegRNAs). For example, in various embodiments a first pegRNA modifies (e.g., is engineered to modify or physically modifies) an EpoR-encoding nucleic acid sequence and a second pegRNA modifies a therapeutic target, e.g., a gene comprising a genetic lesion associated with a disease, disorder, or condition. In various embodiments, a first pegRNA modifies one or more nucleotide positions at a first locus within an EpoR-encoding nucleic acid and a second pegRNA modifies one or more different nucleotide positions at a second different locus within an EpoR-encoding nucleic acid, optionally where at least a third pegRNA can modify a therapeutic target.

III(A)(1)(c). CRISPR Payload Expression Products for Modification of Endogenous Nucleic Acids Encoding EpoR and/or Other Expression Products

A payload expression product of the present disclosure can be a CRISPR editing system or one or more components thereof. In various embodiments, the present disclosure includes editing systems that utilize CRISPR editing system for editing of nucleic acid targets, including in various embodiments modification of an EpoR-encoding nucleic acid to produce a modified nucleic acid encoding signaling-enhanced EpoR. The present disclosure includes, among other things, CRISPR editing agents and systems, and nucleic acids encoding the same, e.g., where the nucleic acid is present in a nucleic acid payload. A CRISPR editing system can include a CRISPR editing enzyme and/or at least one gRNA as components thereof. The CRISPR (Clustered Regularly Interspaced Short Palindromic Repeats)/Cas (CRISPR-associated protein) nuclease system is an engineered nuclease system used for genetic engineering that is based on a bacterial system. It is based in part on the adaptive immune response of many bacteria and archaea. When a virus or plasmid invades a bacterium, segments of the invader's DNA are converted into CRISPR RNAs (crRNA) by the bacteria's “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a 20-nucleotide complementary strand sequence contained within the crRNA transcript. In some instances, the Cas nuclease requires both the crRNA and the tracrRNA for site-specific DNA recognition and cleavage.

Guide RNAs (gRNAs) are an example of an element that can target CRISPR editing. In its simplest form, gRNA provides a sequence that targets a site within a genome based on complementarity (e.g., crRNA). As explained below, however, gRNA can also include additional components. For example, in particular embodiments, gRNA can include a targeting sequence (e.g., crRNA) and a component to link the targeting sequence to a cutting element. This linking component can be tracrRNA. In particular embodiments, gRNA including crRNA and tracrRNA can be expressed as a single molecule referred to as single gRNA (sgRNA). gRNA can also be linked to a cutting element through other mechanisms such as through a nanoparticle or through expression or construction of a dual or multi-purpose molecule. Those of skill in the art will appreciate that gRNA or other targeting elements that can be used to generate a selected nucleic acid sequence correction or modification, e.g., in a host cell of a nucleic acid payload of the present disclosure, can be readily designed and implemented, e.g., based on available sequence information.

In particular embodiments, targeting elements (e.g., gRNA) can include one or more modifications (e.g., a base modification, a backbone modification), to provide the nucleic acid with a new or enhanced feature (e.g., improved stability). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Examples of cutting elements include nucleases. CRISPR-Cas loci have more than 50 gene families and there are no strictly universal genes, indicating fast evolution and extreme diversity of loci architecture. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, CasY, Cpf1, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx100, Csx16, CsaX, Csx3, Csx1, Csx15, Csf1, Csf2, Csf3, Csf4, and variants thereof.

There are three main types of Cas nucleases (type I, type II, and type III), and 10 subtypes including 5 type I, 3 type II, and 2 type III proteins (see, e.g., Hochstrasser and Doudna, Trends Biochem Sci, 2015: 40 (1): 58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NCBI Accession No. NP_269215, and the amino acid sequence of Streptococcus thermophilus wild-type Cas9 polypeptide is set forth, e.g., in NCBI Accession No. WP_011681470.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species.

In some embodiments, crRNA and tracrRNA can be combined into one molecule called a single gRNA (sgRNA). In this engineered approach, the sgRNA guides Cas to target any desired sequence (see, e.g., Jinek et al., Science 337:816-821, 2012; Jinek et al., eLife 2: e00471, 2013; Segal, eLife 2: e00563, 2013). Thus, the CRISPR/Cas system can be engineered to create a double-strand break at a desired target in a genome of a cell, and harness the cell's endogenous mechanisms to repair the induced break by HDR, or NHEJ. Particular embodiments described herein utilize homology arms to promote HDR at defined integration sites.

In various embodiments, variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863 A. A double-strand break is introduced using a Cas9 nickase if at least two DNA-targeting RNAs that target opposite DNA strands are used. A double-nicked induced double-strand break is repaired by HDR or NHEJ. This gene editing strategy generally favors HDR and decreases the frequency of indel mutations at off-target DNA sites. The Cas9 nuclease or nickase, in some embodiments, is codon-optimized for the target cell or target organism.

III(A)(1)(d). Zinc finger nucleases for modification of endogenous nucleic acids encoding EpoR and/or other expression products

A payload expression product of the present disclosure can be a Zinc Finger Nuclease. Zinc finger nucleases (ZFNs) are artificial restriction enzymes made by associating a sequence-targeted zinc-finger DNA-binding unit with a nuclease domain (e.g., Fok1 nuclease domain) in a fusion protein. Each ZFN includes a nuclease domain (e.g., the cleavage domain of FokI) linked to an array of three to six zinc fingers (ZFs). For example, a ZFN can include several CyszHisz ZFs in which each unit includes about 30 amino acids and specifically binds about 3 nucleotides. The ZFs provide a ZFN with the ability to bind a particular nucleic acid sequence. Because the FokI cleavage domain must dimerize to cut DNA, a monomer is not active, and cleavage does not occur at single binding sites. Thus, for example, ZFNs including three ZFs that together bind a 9-bp target function as ZEN dimers that specifically bind 18 bp of DNA per cleavage site. In some embodiments, ZFNs can include up to six ZFs per ZFN.

Cleavage of a target nucleic acid by a ZFN induces cellular repair processes that can mediate modification of the nucleic acid. ZFN-induced double-strand breaks can lead to both targeted modification and targeted gene replacement. For example, if a ZFN-induced cleavage is resolved by non-homologous end joining, this can result in small deletions or insertions, which can lead to gene knockout. If a ZFN-induced cleavage is resolved by a homology-based process in the presence of a provided donor nucleic acid, small changes (e.g., one or a few nucleotides) or more (e.g., up to and including entire transgenes) can be introduced into the target nucleic acid.

III(A)(1)(e). TALENs for modification of nucleic acids for modification of endogenous nucleic acids encoding EpoR and/or other expression products

A payload expression product of the present disclosure can be a Transcription Activator-Like Effector Nuclease (TALEN) editing systems. Various editing enzymes and systems can include a transcription activator-like (TAL) effector DNA binding domain and an endonuclease enzyme. An editing enzyme including a TAL effector DNA binding domain and an endonuclease can be referred to as a TALEN.

TAL effector DNA binding domains includes a plurality of monomers, each of which monomers binds one nucleotide in the target nucleic acid sequence. Each monomer includes 34 amino acids. In each monomer, positions 12 and 13 (referred to as the repeat variable diresidue, RVD) are highly variable and contribute to specific recognition of different nucleotides. The final monomer of a TAL effector DNA binding domain, which binds the nucleotide at the 3′-end of the recognition site, can be only 20 amino acids in length and therefore is sometimes referred to as a half-repeat. RVD sequences can be degenerate, as certain RVD combinations can bind to two or more nucleotides, e.g., with distinct efficiency. For example, RVDs include Asn and Ile (NI), Asn and Gly (NG), Asn and Asn (NN), and His and Asp (HD), which bind A, T, G, and C nucleotides, respectively.

In various embodiments, a TAL effector DNA binding domain is isolated from Xanthomonas spp. In various embodiments, a TALEN includes an endonuclease domain (e.g., a FokI domain), e.g., C-terminal to the TAL effector DNA binding domain.

TALENs work as pairs, the two members having target binding site on opposite DNA strands of the target nucleic acid sequence, with the targets separated by a small fragment (e.g., 12-25 bp) that can be referred to as a spacer sequence. Once a pair of TALENs have bound their target sites, the endonuclease (e.g., FokI) domains dimerize and cause a double-strand break in a spacer sequence. Non-homologous end joining (NHEJ) to resolve a DSB directly ligates DNA from either side of the double-strand break where there is very little or no sequence overlap for annealing. This repair mechanism can cause indels (insertion or deletion), or chromosomal rearrangement, which can disrupt genes at that target nucleic acid sequence. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes

### III(A)(2). Signaling-Enhanced EpoR Transgenes

The present disclosure includes embodiments in which a nucleic acid sequence that encodes a signaling-enhanced EpoR of the present disclosure is provided to a cell (e.g., an HSC and/or erythroid progenitor) in the form of a heterologous transgene. In certain particular embodiments, methods and compositions of the present disclosure include a nucleic acid payload that includes a heterologous transgene that encodes signaling-enhanced EpoR. In various embodiments, the transgene encoding the signaling-enhanced EpoR can have the sequence of any signaling-enhanced EpoR-encoding nucleic acid provided herein and/or encode any signaling-enhanced EpoR provided herein. Further, the transgene coding sequence can be operably linked with regulatory elements provided herein, including without limitation a promoter, e.g., a promoter disclosed herein.

### III(A)(3). Binding Domain, Antibody, CAR, and TCR Payload Expression Products

The present disclosure includes payload expression products that include a variety of binding domains. Sequences that encode binding domains can encode, for example, antibodies, chimeric antigen receptors, TCRs, or other binding polypeptides.

Antibodies and antibody fragments are exemplary of binding domains. The term “antibody” can refer to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is polyclonal, monoclonal, monospecific, or multispecific antibodies (including bispecific antibodies). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F (ab′) 2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-Bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

The term “antibody fragment” can refer to a portion of an antibody or antibody agent as described herein, and typically refers to a portion that includes an antigen-binding portion or variable region thereof. An antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In various embodiments, a payload can encode a binding agent that is a checkpoint inhibitor such as an antibody that specifically binds an immune checkpoint protein. A number of immune checkpoint inhibitors are known. Immune checkpoint inhibitors can include peptides, antibodies, nucleic acid molecules and small molecules. Examples of immune checkpoints include PD-1, PD-L1, lymphocyte activation gene-3 (LAG-3), and T cell immunoglobulin and mucin domain-containing molecule 3 (TIM-3).

In various embodiments, a payload can encode an antibody or other binding domain that binds CD4, CD5, CD7, CD52, IL1, IL2, IL6, TCRs specifically present on autoreactive T cells, IL4, IL10, IL12, IL13, ILIRa, sIL1RI, sIL1RII, TNF, ABCA3, ABCD1, ADA, AK2, APP, arginase, arylsulfatase A, A1AT, CD3D, CD3E, CD3G, CD3Z, CFTR, CHD7, chimeric antigen receptor (CAR), CIITA, CLN3, complement factor, CORO1A, CTLA, C1 inhibitor, C9ORF72, DCLREIB, DCLRE1C, decoy receptors, DKC1, DRB1*1501/DQB1*0602, dystrophin, enzymes, Factor VIII, FANC family genes (FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), Fas L, FUS, GATA1, globin family genes (ie. γ-globin), F8, glutaminase, HBA1, HBA2, HBB, IL7RA, JAK3, LCK, LIG4, LRRK2, NHEJ1, NLX2.1, ORAI1, PARK2, PARK7, phox, PINK1, PNP, PRKDC, PSEN1, PSEN2, PTPN22, PTPRC, P53, pyruvate kinase, RAG1, RAG2, RFXANK, RFXAP, RFX5, RMRP, ribosomal proteins, SFTPB, SFTPC, SOD1, soluble CD40, STIM1, STNFRI, STNFRII, SLC46A1, SNCA, TDP43, TERT, TERC, TINF2, ubiquilin 2, WAS, WHN, ZAP70, γC, and other payload expression products described herein.

Payload expression products can include chimeric antigen receptors (CARs). HSCs and/or erythroid progenitors can be engineered to encode and/or express CAR constructs. CARs can include several distinct subcomponents that can cause cells to recognize and kill target cells such as cancer cells. In various embodiments, the subcomponents can include at least an extracellular component, a transmembrane domain, and an intracellular component.

An extracellular CAR component can include a binding domain that specifically binds a marker that is preferentially present on the surface of unwanted cells. When the binding domain binds such markers, the intracellular component directs a cell to destroy the bound cancer cell. The binding domain is typically a single-chain variable fragment (scFv) derived from a monoclonal antibody (mAb), but it can be based on other formats which include an antibody-like antigen binding site.

Intracellular CAR components provide activation signals based on the inclusion of an effector domain. First generation CARs utilized the cytoplasmic region of CD3ζ as an effector domain. Second generation CARs utilized CD32 in combination with cluster of differentiation 28 (CD28) or 4-1BB (CD137), while third generation CARs have utilized CD3ζ in combination with CD28 and 4-1BB within intracellular effector domains.

Intracellular or otherwise cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from a co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKD80 (KLRF1), CD127, CD160, CD19, CD4, CD8a, CD8B, IL2RB, IL2Ry, IL7Ra, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKD44, NKD30, or NKD46.

Intracellular signaling component sequences that act in a stimulatory manner may include ITAMs. Examples of ITAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3 chain, and/or co-receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119 (3): 696-706). Further examples of such co-stimulatory domain molecules include CDS, ICAM-1, GITR, BAFFR, HVEM (LIGHTR), SLAMF7, NKD80 (KLRF1), NKD44, NKD30, NKD46, CD160, CD19, CD4, CD8a, CD8B, IL2RB, IL2Ry, IL7Ra, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11lc, ITGB1, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD33 and a portion of the 4-1BB intracellular signaling component.

In particular embodiments, the intracellular signaling component includes (i) all or a portion of the signaling domain of CD3, (ii) all or a portion of the signaling domain of 4-1BB, or (iii) all or a portion of the signaling domain of CD33 and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family), G-protein-coupled receptors, GPCRs (Frizzled or Smoothened), serine/threonine kinase receptors (BMPR or TGFR), or cytokine receptors (ILIR, IL2R, IL7R, or IL15R).

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. A common spacer region used for this purpose is the IgG4 linker. More compact spacers or longer spacers can be used, depending on the targeted cell marker. Other potential CAR subcomponents are described in more detail elsewhere herein.

Transmembrane domains within a CAR molecule, often serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the a, B or (chain of a T-cell receptor, CD28, CD27, CD3 epsilon, CD45, CD4, CD5, CD8, CD9, CD16, CD22, CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKD80 (KLRF1), NKD44, NKD30, NKD46, CD160, CD19, IL2RB, IL2RY, IL7R a, ITGA1, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

TCRs refer to naturally occurring T cell receptors. Payloads of the present disclosure can encode a TCR or a CAR/TCR hybrid that includes an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

### III(A)(4). Small RNA Payload Expression Products

A payload expression product of the present disclosure can be a small RNA. Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50, 45, 40, 35, 30, 25, or 20 nucleotides in length. In particular embodiments, small RNAs are less than 20 nucleotides in length. In various embodiments, a small RNA has a length having a lower bound of 5, 10, 15, 20, 25, or 30 nucleotides and an upper bound of 20, 25, 30, 35, 40, 45, 50, 75, or 100 nucleotides. Small RNAs include but are not limited to microRNAs (miRNAs, Piwi-interacting RNAs (piRNAs), small interfering RNAs (siRNAs), small nucleolar RNAs (snoRNAs), tRNA-derived small RNAs (tsRNAs) small rDNA-derived RNAs (srRNAs), and small nuclear RNAs. Additional classes of small RNAs continue to be discovered.

In particular embodiments, interfering RNA molecules that are homologous to a target mRNA or to which the interfering RNA can hybridize can lead to degradation of the target mRNA molecule or reduced translation of the target mRNA, a process referred to as RNA interference (RNAi) (Carthew, Curr. Opin. Cell. Biol. 13:244-248, 2001). RNAi occurs in cells naturally to remove foreign RNAs (e.g., viral RNAs). In some instances, natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference in nature and/or in some embodiments is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs.

In a second step, an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA. Research indicates that each RISC contains a single siRNA and an RNase.

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III(Pol III) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides.

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp, 50-60 bp, or 51, 52, 53, 54, 55, 56, 57, 58, or 59 bp. In particular embodiments, shRNA sequences include 52 or 55 bp. In particular embodiments, siRNAs have 15-25 bp. In particular embodiments, siRNAs have 16, 17, 18, 19, 20, 21, 22, 23, or 24 bp. In particular embodiments, siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

In certain illustrative embodiments, the present disclosure includes a nucleic acid payload that encodes an shRNA targeted to the gene encoding BCL11A, where the shRNA causes decreased translation of BCL11A.

### III(A)(5). Selection Sequences

In various embodiments, in addition to including a nucleic acid sequence that encodes and/or expresses signaling-enhanced EpoR, a nucleic acid payload of the present disclosure can further include a nucleic acid sequence that encodes and/or expresses an agent that enables selection of modified cells (a selectable marker) by conferring to the modified cell resistance to a selecting agent or selecting condition. In particular embodiments, a selectable marker is encoded by and/or expressed from a selection cassette that includes a promoter, a cDNA that adds or confers resistance to a selection agent, and/or a poly A sequence.

In various embodiments, a nucleic acid payload includes a transgene that encodes the selectable marker MGMT^{P140K}. MGMT is a DNA repair enzyme that can repair damaged guanine nucleotides by transferring the methyl at the 06 site of guanine to its cysteine residues, which can counteract the genotoxicity of alkylating agents. A reference MGMT polypeptide can have a sequence according to GenBank Accession No. NP_002403.3. The present disclosure includes certain variants of MGMT that are resistant to MGMT inhibitors. Exposure to MGMT inhibitors can sensitize cells that express wild type MGMT to elimination by alkylating agents. MGMT functions, at least in part, as a DNA repair enzyme that protects cells from alkylating agents. However, the protective functions of wild type MGMT are antagonized by MGMT inhibitors (e.g., O6-benzylguanine (O6BG), Lomeguatrib [6-(4-bromo-2-thienyl) methoxy]purin-2-amine], and others, rendering cells vulnerable to elimination by alkylating agents. In various embodiments, an inhibitor-resistant MGMT is an MGMT polypeptide that includes the mutation P140K (e.g., MGMTP140K). In various embodiments, cells that encode and/or express transgenic inhibitor-resistant MGMT are less likely to be eliminated by a selection regimen that includes one or more MGMT inhibitors and optionally one or more alkylating agents. Accordingly, administration of a selection regimen including one or more MGMT inhibitors and optionally one or more alkylating agents can positively select modified cells (e.g., MGMT-modified cells).

In various embodiments, an MGMT inhibitor is or includes O⁶-meG or an analog or derivative thereof. In various embodiments, an MGMT inhibitor is or includes O⁶-benzylguanine (O⁶BG) or an analog or derivative thereof. O⁶BG donates an alkyl group to MGMT, inactivating it and initiating degradation. In various embodiments, an analog or derivative of O⁶-meG and/or O⁶BG can inhibit MGMT through alkyl group transfer. In various embodiments, an analog or derivative of O⁶-meG and/or O⁶BG can be or include O⁶-(3-bromobenzyl) guanine, O⁶-2-fluoropyridinylmethylguanine (O⁶FPG), O6-3-iodobenzylguanine (O⁶IBG), O⁶-(4-bromothenyl) guanine (O⁶BTG; PaTrin-2), O⁶-5-iodothenylguanine (O⁶ITG), 8-aza-O6-benzylguanine (8-aza-BG), O⁶-benzyl-8-bromoguanine (8-bromo-BG), 2-amino-4-benzyloxy-5-nitropyrimidine (4-desamino-5-nitro-BP), O⁶-[p-(hydroxymethyl)benzyl] guanine (HN-BG), O⁶-benzyl-8-methylguanine (8-methyl-BG), O⁶-benzyl-7, 8-dihydro-8-oxoguanine (8-oxo-BG), 2,4,5,-triamino-6-benzyloxyprimidine (5-amino-BP), O⁶-benzyl-9-[(3-oxo-5 α-androstan-17β-yloxycarbonyl)methyl] guanine (DHT-BG), O⁶-benzyl-9-(3-oxo-4-androsten-17β-yloxycarbonyl)methyl] guanine (AND-BG), and/or 8-amino-O⁶-benzylguanine (8-amino-BG). In various embodiments, an MGMT inhibitor is or includes diethylamine NONOate, Lomeguatrib, 2,4-diamino-6-benzyloxy-5-nitrosopyrimidine (5-nitroso-BP), and/or 2,4-diamino-6-benzyloxy-5-nitropyrimidine (5-nitro-BP).

Various alkylating agents are known in the art. Alkylating agents include, without limitation, a nitrosoureas (e.g., nimustine (ACNU), carmustine (bis-chloroethylnitrosourea; BCNU), lomustine (CCNU), streptozocin, or semustine (methyl CCNU)), a nitrogen mustard (e.g., mechlorethamine, cyclophosphamide, ifosfamide, melphalan, chlormethine, ramustine or uracil mustard, bendamustine, or chlorambucil), ethylenamine and methylenamine derivatives (e.g., altretamine, thiotepa), an aziridine or epoxide (e.g., thiotepa, mitomycin C, or diaziquone (AZQ)), an alkyl sulfonate (e.g., busulfan or hepsulfam), a triazine or hydrazine (e.g., mitozolomide, temozolomide, procarbazine or dacarbazine), hexamethylmelamine, melphalan, and chlorambucil. As one example of an alkylating agent, BCNU promotes DNA alkylation at the 06 position of guanine, leading to DNA interstrand crosslinking and altered fidelity of DNA replication and transcription. This induced interstrand crosslinking involves formation of a chloroethyl adduct at the guanine residue that undergoes an intramolecular rearrangement to produce an unstable intermediate that reacts with the cross strand cytosine residue. The result is an N′-guanine, N3-cytosine-ethanol crosslink.

Examples of selectable markers can include one or more proteins that (a) confer resistance to antibiotics or other toxins, (b) complement auxotrophic deficiencies, or (c) supply critical nutrients not available from complex media, e.g., the gene encoding D-alanine racemase for Bacilli. In particular embodiments, a selectable marker for positive selection can be a protein that confers resistance to neomycin, hygromycin, ampicillin, puromycin, phleomycin, zeomycin, blasticidin, or viomycin. In particular embodiments, a selectable marker for positive selection can be DHFR (dihydrofolate reductase; providing resistance to methotrexate), MGMT^{P140K} (providing resistance to O⁶BG/BCNU), HPRT (Hypoxanthine phosphoribosyl transferase; transformation of specific bases present in the HAT selection medium (aminopterin, hypoxanthine, thymidine)), and other proteins for detoxification of particular drugs. In various appropriate embodiments, a selecting agent can be neomycin, hygromycin, puromycin, phleomycin, zeomycin, blasticidin, viomycin, ampicillin, O⁶BG/BCNU, methotrexate, tetracycline, aminopterin, hypoxanthine, thymidine kinase, DHFR, Gln synthetase, or ADA.

In various embodiments, a selectable marker for negative selection can be an expression product that transforms a substrate present in (e.g., delivered to) a subject or system (e.g., a culture medium) into a toxic substance, thereby sensitizing cells that expresses the selectable marker. In various embodiments, for example, a nucleic acid payload is engineered such that proper integration of all or a portion of the payload into a target genome disrupts expression of the gene encoding a negative selectable marker. A negative selectable marker can include a diptheria toxin A-fragment (DTA) (Yagi et al., Anal Biochem. 214 (1): 77-86, 1993; Yanagawa et al., Transgenic Res. 8 (3): 215-221, 1999) or a thymidine kinase of the Herpes virus (HSV TK) (sensitive to the presence of ganciclovir or FIAU). In various embodiments, a negative selectable marker can include HPRT (negative selection in the presence of 6-thioguanine (6TG)).

In particular embodiments, a selectable marker is MGMT^{P140K} (see, e.g., Olszko Gene Therapy 22:591-595, 2015). In particular elements, the selecting agent includes O⁶BG/BCNU. The MGMT gene encodes human alkyl guanine transferase (hAGT), a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG but retain their ability to repair DNA damage (Maze et al., J. Pharmacol. Exp. Ther. 290:1467-1474, 1999). MGMT^{P140K} has been shown to confer selection in subjects and systems including mouse, canine, rhesus macaques, and human cells, specifically hematopoietic cells (Zielske et al., J. Clin. Invest. 112:1561-1570, 2003; Pollok et al., Hum. Gene Ther. 14:1703-1714, 2003; Gerull et al., Hum. Gene Ther. 18:451-456, 2007; Neff et al., Blood 105:997-1002, 2005; Larochelle et al., J. Clin. Invest. 119:1952-1963, 2009; Sawai et al., Mol. Ther. 3:78-87, 2001).

In particular embodiments, in vivo selection can be useful to increase efficacy of gene therapy for diseases in which therapeutically modified cells are not otherwise conferred a selective advantage. For example, in SCID and some other immunodeficiencies and FA, corrected cells have an advantage and only transducing a condition-corrective nucleic acid payload into a “few” HSCs and/or erythroid progenitors is sufficient for therapeutic efficacy without further selection. For other diseases like hemoglobinopathies (i.e., sickle cell disease and thalassemia) in which therapeutically modified cells are not understood to confer a competitive advantage, in vivo selection of the modified cells, e.g., based on inclusion of a nucleic acid encoding a selectable marker such as MGMT^{P140K} together with a therapeutic gene in a therapeutic nucleic acid payload, selects for the initially transduced cells and causes an increase in the prevalence of cells carrying the therapeutic nucleic acid payload, improving therapeutic efficacy.

### III(B). Payload Regulatory Sequences

A payload expression product of the present disclosure can be expressed from a coding sequence that is operably linked with one or more regulatory sequences, including without limitation a promoter, enhancer, insulator, termination signal, polyadenylation signal, splicing signal, and/or the like. Those of skill in the art will appreciate that methods and techniques for operably linking a regulatory sequence and a coding sequence are known. Various exemplary regulatory sequences, such as exemplary promoters, are provided herein by way of example.

Promoters can include general promoters, tissue-specific promoters, cell-specific promoters, and/or promoters specific for the cytoplasm, examples of each of which are well known in the art. Promoters may include strong promoters, weak promoters, constitutive expression promoters, and/or inducible (conditional) promoters. Inducible promoters direct or control expression in response to certain conditions, signals, or cellular events. For example, a promoter can be an inducible promoter that requires a particular ligand, small molecule, transcription factor, hormone, or hormone protein in order to effect transcription from the promoter

In various embodiments, a promoter sequence can be a native promoter sequence. A native promoter sequence, or minimal promoter sequence, can refer to a sequence derived from a single contiguous sequence positioned 5′ of a coding sequence in a reference genome. A native promoter sequence can include a core promoter and an associated 5′UTR. In particular embodiments, a 5′UTR can include an intron. In various embodiments, a promoter sequence can be a composite promoter sequence. In various embodiments, a composite promoter sequence can refer to a promoter sequence that includes portions derived from at least two distinct sources, e.g., from two non-contiguous portions of a reference genome, from two distinct genomes, or from any two distinct source sequences. For example, in certain embodiments, a composite promoter sequence includes a sequence derived from a single contiguous sequence positioned 5′ of a coding sequence in a reference genome and a sequence derived from another portion of the reference genome, e.g., an enhancer (e.g., a distal enhancer).

In particular embodiments, a promoter can be a wild type promoter sequence or a sequence with one or more changes relative to a reference promoter (e.g., one or more insertions, point mutations, or deletions). In particular embodiments, a promoter sequence differs from a wild type or other reference promoter sequence by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, a promoter sequence can differ from a wild type or reference sequence by 1, 2, 3, 4, 5, 6, 7, 8, 9, or 10 nucleotide differences. A promoter can have a length of, e.g., 50 to 3,000 or more nucleotides, e.g., 100-1,000, 100-2,000, 100-3,000, 500-1,000, 500-2,000, 500-3,000, 1,000-2,000, or 1,000-3,000 nucleotides.

Particular examples of promoters include the AFP (α-fetoprotein) promoter, amylase 1C promoter, aquaporin-5 (AP5) promoter, αl-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, COX-2 promoter, CXCR4 promoter, desmin promoter, E2F-1 promoter, human elongation factor 1α promoter (EF1α), CMV (cytomegalovirus viral) promoter, minCMV promoter, SV40 (simian virus 40) immediately early promoter, EGR1 promoter, eIF4A1 promoter, elastase-1 promoter, endoglin promoter, FerH promoter, FerL promoter, fibronectin promoter, Flt-1 promoter, GAPDH promoter, GFAP promoter, GPIIb promoter, GRP78 promoter, GRP94 promoter, HE4 promoter, hGR1/1 promoter, hNIS promoter, Hsp68 promoter, the Hsp68 minimal promoter (proHSP68), HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, NphsI promoter, OG-2 promoter, PGK (Phospho Glycerate kinase) promoters, PGK-1 promoter, polymerase III(Pol III) promoter, PSA promoter, ROSA promoter, SP-B promoter, Survivn promoter, SYN1 promoter, SYT8 gene promoter, TRP1 promoter, Tyr promoter, ubiquitin B promoter, WASP promoter, and the Rous Sarcoma Virus (RSV) long-terminal repeat (LTR) promoter.

In some embodiments, the promoter is a non-specific promoter. In particular embodiments, a non-specific promoter includes CMV promoter, RSV promoter, SV40 promoter, mammalian elongation factor 1α (EF1α) promoter, β-act promoter, EGR1 promoter, eIF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

In various embodiments, a promoter is non-specific in that it causes expression of an operably linked coding sequence in cells or tissues of diverse types. In various embodiments, a promoter is a ubiquitous promoter. In various embodiments, a ubiquitous promoter can be selected from, e.g., a CMV promoter, RSV promoter, or SV40 promoter.

In various embodiments, a coding sequence is operably linked to a microRNA (or miRNA) control system. An miRNA control system can refer to a method or composition in which expression of a coding sequence is regulated by the presence of microRNA sites (e.g., nucleic acid sequences with which a microRNA can interact). In various embodiments, the present disclosure includes payload in which a nucleic acid sequence encoding an expression product is operably linked to an miRNA target site such that expression of the expression product is controlled by presence, level, activity, and/or contact with a corresponding miRNA. For the avoidance of doubt, the present disclosure contemplates that a nucleic acid sequence operably linked with an miRNA site, e.g., as disclosed herein can be a nucleic acid sequence that encodes, e.g., any of one or more expression products provided herein.

Coding sequences of the present disclosure can additionally be associated with sequences that enhance the stability of mRNA transcripts, such as an insulator and/or a polyA tail.

### III(C). Integrating and Non-Integrating Nucleic Acid Payload Elements

In various embodiments, a nucleic acid payload of the present disclosure includes a fragment that integrates (or is engineered to integrate) into genomic DNA of a target or recipient subject, cell, or system. Like other adenoviral vectors, typical HDAd genomes generally remain episomal and do not integrate with a host genome (e.g., other than where engineered to include an integrating fragment). The present disclosure includes that, in various embodiments, integration of all, or an integrating fragment of, a nucleic acid payload into the genome of a target cell contributes substantially to effective gene therapy. The present disclosure also includes that, in various embodiments, non-integration of all, or a non-integrating fragment of, a nucleic acid payload into the genome of a target cell contributes substantially to effective gene therapy. A variety of systems can be designed and/or used for integration of a nucleic acid into a host or target cell genome. Various such systems can include one or more of certain payload sequence features that cause (or prevent) integration, and can further include support vectors and support genomes (support genomes) as further disclosed herein.

In various embodiments, a payload can include a nucleic acid sequence engineered for integration into a host cell genome (an “integrating payload”), e.g., by recombination or transposition. In various embodiments, a payload can include a nucleic acid sequence that is engineered for integration into a host cell genome in that it is flanked by sequences for recombination with genomic DNA and/or for transposition into genomic DNA (e.g., is not flanked by transposon inverted repeats). In various embodiments, a nucleic acid payload of the present disclosure includes a fragment that does not integrate (and/or is not engineered to integrate) into genomic DNA of a target or recipient subject, cell, or system (a “non-integrating payload”). In various embodiments, a payload can include a nucleic acid sequence that is not engineered for integration into a host cell genome in that it is not flanked by sequences for recombination with genomic DNA and/or transposition into genomic DNA (e.g., is not flanked by transposon inverted repeats). In various embodiments, a payload this is not specifically associated with and/or engineered to include sequences that cause integration into genomic DNA can be referred to as a non-integrating payload, e.g., when present in a vector or vector nucleic acid sequence (e.g., a viral vector genome) that is not characterized by an ability to integrate into genomic DNA. A nucleic acid payload of the present disclosure can include an “integrating” portion that is engineered to integrate into genomic DNA of a target or recipient subject, cell, or system and a “non-integrating” portion that is not engineered to integrate into genomic DNA of a target or recipient subject, cell, or system.

In various embodiments, a fragment of a nucleic acid payload that encodes an editing system engineered to modify an endogenous EpoR nucleic acid to produce a modified nucleic acid encoding signaling-enhanced EpoR (an “EpoR editing payload”) is present in a nucleic acid payload that includes at least one therapeutic payload (e.g., a therapeutic payload that does not encode an editing system), where the therapeutic payload is integrating and the EpoR editing payload is non-integrating. In various embodiments, an integrating payload can encode one or more expression products for which permanent, long-term, or lineage-enduring expression is desired. For example, a gene that provides an expression product that counteracts a deficiency of endogenous cells (e.g., an enzyme deficiency and/or a deficiency resulting from a genomic mutation and/or a deficiency associated with a disease, disorder, or condition) can be included in an integrating fragment of a nucleic acid payload of the present disclosure. In various embodiments, an editing enzyme or editing system of the present disclosure is encoded by a non-integrating portion of a nucleic acid payload of the present disclosure, e.g., to minimize the level and/or duration of expression and/or activity of an encoded agent, and, where applicable, genotoxicity, fitness costs, or other undesired effects resulting the One means of engineering a nucleic acid payload that integrates into a host cell genome is to include genetic elements known (e.g., naturally utilized by one or more organisms) to cause stable integration of associated nucleic acids into a new nucleic acid context (e.g., into a host cell genome and/or different position within the same genome). The present disclosure also includes polypeptides that act upon certain such genetic elements to cause integration. In various embodiments, an “integration system” can include a polypeptide that acts upon (or “targets”) certain genetic elements to cause integration of associated nucleic acid sequences, and the genetic elements targeted by the polypeptide, e.g., when present in a nucleic acid payload. In various embodiments, an “integration system” can include certain genetic elements that, when introduced into a target cell, are sufficient to cause integration of associated nucleic acid sequences without further introduction (e.g., transduction) into the target cell of a polypeptide. Integration systems of the present disclosure can include, e.g., bacteriophage integrase PHiC31, a retrotransposon, a retrovirus (e.g., LTR-mediated or retrovirus integrate-mediated), a zinc-finger nuclease, a DNA-binding domain-retroviral integrase fusion protein, an AAV sequence (e.g., an AAV-ITR or sequence for AAV-Rep protein-mediated integration), or a transposase (e.g., a Sleeping Beauty (SB) transposase). Those of skill in the art will appreciate that any of these exemplary integration systems, and others known in the art, can be used to engineer an integrating payload.

The present disclosure includes that in various embodiments a nucleic acid payload can be engineered to a position a fragment of the payload between transposase target sites (transpose inverted terminal repeats; “ITRs”), such that the flanked fragment can be transposed into the genome of a host cell in the presence of a corresponding transposase. A transposition reaction includes a transposon and a transposase or an integrase enzyme. Transposons include terminal repeat sequences upstream and downstream of a larger segment of DNA. Transposases bind the terminal repeat sequences and catalyze movement of the transposon form a first nucleic acid context to a different nucleic acid context. Transposases can include integrases from retrotransposons or of retroviral origin, as well as an enzyme that is a component of a functional nucleic acid-protein complex capable of transposition.

A number of transposases have been described in the art that facilitate insertion of nucleic acids into the genome of vertebrates, including humans. Examples of such transposases include sleeping beauty (“SB”, e.g., derived from the genome of salmonid fish), piggyback (e.g., derived from lepidopteran cells and/or the Myotis lucifugus), mariner (e.g., derived from Drosophila), frog prince (e.g., derived from Rana pipiens), Tol1 or Tol2 (e.g., derived from medaka fish), TcBuster (e.g., derived from the red flour beetle Tribolium castaneum), Helraiser, Himar1, Passport, Minos, Ac/Ds, PIF, Harbinger, Harbinger3-DR, HSmar1, and spinON.

The PiggyBac (PB) transposase is a compact functional transposase protein that is described in, for example, Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

Additional information on DNA transposons can be found, for instance, in Muñoz-López & García Pérez, Curr Genomics, 11 (2): 115-128, 2010.

Sleeping Beauty transpose systems are described, e.g., in Ivics et al. Cell 91, 501-510, 1997; Izsvak et al., J. Mol. Biol., 302 (1): 93-102, 2000; Geurts et al., Molecular Therapy, 8 (1): 108-117, 2003; Mates et al. Nature Genetics 41:753-761, 2009; and U.S. Pat. Nos. 6,489,458; 7,148,203; and 7,160,682; US Publication Nos. 2011/117072; 2004/077572; and 2006/252140. SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase. Systematic mutagenesis studies have been undertaken to increase the activity of the SB transposase. For example, Yant et al., undertook the systematic exchange of the N-terminal 95 AA of the SB transposase for alanine (Mol. Cell Biol. 24:9239-9247, 2004). Ten of these substitutions caused hyperactivity between 200-400% as compared to SB10 as a reference. SB16, described in Baus et al. (Mol. Therapy 12:1148-1156, 2005) was reported to have a 16-fold activity increase as compared to SB10. Additional hyperactive SB variants are described in Zayed et al. (Molecular Therapy 9 (2): 292-304, 2004) and U.S. Pat. No. 9,840,696. In certain embodiments, the Sleeping Beauty transposase enzyme is a Hyperactive Sleeping Beauty SB100x transposase enzyme. SB transposons are most efficiently transposed when present in circularized nucleic acid molecules (Yant et al., Nature Biotechnology, 20:999-1005, 2002).

In certain exemplary embodiments, a nucleic acid payload includes a fragment that includes SB 100x transposon inverted repeats that flank a nucleic acid sequence that includes at least one coding sequence that encodes a β-globin expression product or a γ-globin expression product.

In certain embodiments, a nucleic acid payload includes an integrating element engineered to integrate into a host cell genome in the presence of a particular polypeptide (e.g., the transposase of a transposition system including a payload fragment flanked by transposase ITRs). In certain such embodiments, the particular polypeptide may be naturally present in, endogenously expressed by the target cell, or otherwise present in the target cell. In certain embodiments, the particular polypeptide that facilitates or causes integration is delivered to the target cell, e.g., by gene therapy. In certain embodiments, the particular polypeptide that facilitates or causes integration is delivered to the target cell by inclusion of a nucleic acid encoding the particular polypeptide in the same nucleic acid payload that encodes the integrating fragment. In certain embodiments, the particular polypeptide that facilitates or causes integration is delivered to the target cell by inclusion of a nucleic acid encoding the particular polypeptide in a different nucleic acid payload than the nucleic acid payload that encodes the integrating fragment, which further nucleic acid payload is separately administered to a (e.g., the same) recipient subject, cell, or system. A further nucleic acid payload that encodes a particular polypeptide that facilitates or causes integration of an integrating fragment present in another nucleic acid payload can be referred to as a support nucleic acid payload. Thus, for the avoidance of doubt, methods and compositions of the present disclosure can include a first vector that includes or encodes a first nucleic acid payload and a second vector that includes or encodes a second nucleic acid payload, where the first nucleic acid payload includes an integrating element and the second, support nucleic acid payload encodes a polypeptide that facilitates or causes integration of the integrating fragment. Vectors including the nucleic acid payloads can be administered together or separately.

Particular embodiments disclosed herein also use site-specific recombinase systems. In these embodiments, the transposon including transposase-recognized inverted repeats also includes at least one recombinase-recognized site. Thus, in particular embodiments, The present disclosure also provides methods of integrating a nucleic acid payload into the genome including delivering to a target cell: (a) a nucleic acid payload including an integrating fragment that is a transposon flanked by (i) an inverted repeat sequence recognized by a transposase and (ii) recombinase-recognized sites; and b) a transposase and recombinase that serve to excise the nucleic acid payload from a larger nucleic acid in which it is present and integrate the nucleic acid payload into the host cell genome. In some embodiments, the protein(s) of (b) are provided by delivering to the target cell support nucleic acid payload(s) that encode the transposase and recombinase. In some embodiments, the transposon and the nucleic acids encoding the protein(s) of (b) are present on separate vectors. In some embodiments, the transposon and nucleic acid encoding the protein(s) of (b) are present on the same vector. When present on the same vector, the portion of the vector encoding the protein(s) of (b) are located outside the integrating fragment. In other words, the transposase and/or recombinase encoding region is located external to the region flanked by the inverted repeats and/or recombinase-recognition site. In the aforementioned methods, the transposase protein recognizes the inverted repeats that flank an inserted nucleic acid, such as a nucleic acid that is to be inserted into a target cell genome.

Examples of recombinase systems include the Flp/Frt system, the Cre/loxP system, the Dre/rox system, the Vika/vox system, and the PhiC31 system. The Flp/Frt DNA recombinase system was isolated from Saccharomyces cerevisiae. The Flp/Frt system includes the recombinase Flp (flippase) that catalyzes DNA-recombination on its Frt recognition sites. Variants of the Flp protein include GenBank accession no. ABD57356.1 and GenBank accession no. ANW61888.1.

The Cre/loxP system is described in, for example, EP 02200009B1. Cre is a site-specific DNA recombinase isolated from bacteriophage P1. The recognition site of the Cre protein is a nucleotide sequence of 34 base pairs, the loxP site. Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: lox2272, lox511, lox66, lox71, loxM2, and lox5171. The VCre/VloxP recombinase system was isolated from Vibrio plasmid p0908. The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 and the rox recognition site. The Vika/vox system is described in U.S. Pat. No. 10,253,332. Additionally, the PhiC31 recombinase recognizes the AttB/AttP binding sites.

In various embodiments, an integrating fragment is engineered for integration into a target cell genome by homologous recombination. Particular embodiments utilize homology arms to facilitate targeted insertion of an integrating fragment by homology directed repair. Homology arms can be any length with sufficient homology to a genomic sequence at a cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. Homology arms are generally identical to the genomic sequence, for example, to the genomic region in which the double stranded break (DSB) occurs. However, as indicated, absolute identity is not required.

Particular embodiments can utilize homology arms with 25, 50, 100, or 200 nucleotides (nt), or more than 200 nt of sequence homology between a homology-directed repair template and a targeted genomic sequence (or any integral value between 10 and 200 nucleotides, or more). In particular embodiments, homology arms are 40-1000 nt in length. In particular embodiments, homology arms are 500-2500 base pairs, 700-2000 base pairs, or 800-1800 base pairs. In particular embodiments, homology arms include at least 800 base pairs or at least 850 base pairs. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

For additional information regarding homology arms, see Richardson et al., Nat Biotechnol. 34 (3): 339-44, 2016.

In particular embodiments, genetic constructs (e.g., genes leading to expression of a therapeutic product within a cell) are precisely inserted within genomic safe harbors. Genomic safe harbor sites can be intragenic or extragenic regions of the genome that are able to accommodate the predictable expression of newly integrated DNA without substantial adverse effects on the host cell. A useful safe harbor can permit sufficient transgene expression to yield desired levels of the encoded protein. A genomic safe harbor site can be a site at which insertion of an integrating fragment does not substantially and/or substantially detrimentally alter cellular functions. Exemplary methods for identifying genomic safe harbor sites are described in Sadelain et al., Nature Reviews 12:51-58, 2012; and Papapetrou et al., Nat Biotechnol. 29 (1): 73-8, 2011. In particular embodiments, a genomic safe harbor site can be a site that meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, a genomic safe harbor must be >150 kb away from a known oncogene, and/or >30 kb away from a known transcription start site, and/or have no overlap with coding mRNA. In particular embodiments, a genomic safe harbor must be >200 kb away from a known oncogene, and/or >40 kb away from a known transcription start site, and/or have no overlap with coding mRNA. In particular embodiments, a genomic safe harbor must be >300 kb away from a known oncogene, and/or >50 kb away from a known transcription start site, and/or have no overlap with coding mRNA. In particular embodiments, a genomic safe harbor must be >150 kb, >200 kb or >300 kb away from a known transcription start site, and/or have no overlap with coding mRNA, and/or be >40 kb or >50 kb away from a known transcription start site with no overlap with coding mRNA, and/or have 100% homologous between an animal of a relevant animal model and the human genome to permit rapid clinical translation of relevant findings.

In particular embodiments, a genomic safe harbor demonstrates a 1:1 ratio of forward: reverse orientations of lentiviral integration further demonstrating the locus does not impact surrounding genetic material.

Particular genomic safe harbors sites include CCR5, HPRT, AAVS1, Rosa and albumin. See also, e.g., U.S. Pat. Nos. 7,951,925, 8,110,379, U.S. 2008/0159996, U.S. 2010/00218264, U.S. 2012/0017290, U.S. 2011/0265198, U.S. 2013/0137104, U.S. 2013/0122591, U.S. 2013/0177983, and U.S. 2013/0177960 for additional information and options for appropriate genomic safe harbor integration sites.

Various technologies known in the art can be used to direct integration of an integrating fragment at specific genomic loci such as genomic safe harbors. For example AAV-mediated gene targeting, as well as homologous recombination enhanced by the introduction of DNA double-strand breaks using site-specific endonucleases (zinc-finger nucleases, meganucleases, transcription activator-like effector (TALE) nucleases), and CRISPR/Cas systems are all tools that can mediate targeted insertion of foreign DNA at predetermined genomic loci such as genomic safe harbors.

In certain embodiments, integration of an integrating fragment at specific genomic loci such as genomic safe harbors can include homology-directed integration using CRISPR enzyme-mediated cleavage of a target genome. CRISPR enzyme (e.g., Cas9) cleaves double stranded DNA at a site specified by a guide RNA (gRNA). The double strand break can be repaired by homology-directed repair (HDR) when a donor template (such as a payload integrating fragment including left and right homology arms) is present. In various such methods, an integrating fragment is a “repair template” in that it includes left and right homology arms (e.g., of 500-3,000 bp) for insertion into a cleaved target genome. CRISPR-mediated gene insertion can be several orders of magnitude more efficient compared with spontaneous recombination of DNA template, demonstrating that CRISPR-mediated gene insertion can be an effective tool for genome editing. Exemplary methods of homology-directed integration of a nucleic acid sequence into a specified genomic locus are known in the art, e.g., in Richardson et al. (Nat Biotechnol. 34 (3): 339-44, 2016).

To provide a non-limiting example, in certain embodiments a method or composition of the present disclosure includes a viral vector (e.g., an adenoviral vector) where a first vector includes a genome that includes a nucleic acid payload that includes an integrating fragment, and a second viral vector (e.g., an adenoviral vector of the same or different serotype) (a “support vector”) includes a genome (a “support genome”) that encodes (e.g., includes a nucleic acid payload that encodes) a polypeptide that facilitates or causes integration of the integrating fragment. For completeness, a support vector or genome that encoding a polypeptide that facilitates or causes integration of an integrating fragment can further encode one or more additional polypeptides, which can in various embodiments support therapeutic efficacy in other ways, e.g., by causing excision and/or circularization of the other nucleic acid payload.

In certain particular embodiments, a support vector is an adenoviral vector that can include (i) an adenoviral capsid; and (ii) an adenoviral support genome including a nucleic acid sequence encoding a transposase that corresponds to the inverted repeats that flank the integrating fragment. Accordingly, in various embodiments, at least one function of a support vector or support genome can be to encode, express, and/or deliver to a target cell a transposase for transposition of an integrating fragment present in a nucleic acid payload administered to a target cell. For instance, in some embodiments, a nucleic acid payload includes SB100x transposon inverted repeats that flank an integrating fragment that includes at least one coding sequence that encodes a β-globin expression product or a γ-globin expression product, and a support vector or support genome includes a coding sequence that encodes SB100x transposase. In certain embodiments, an integrating fragment is flanked by recombinase direct repeats, e.g., where the integrating fragment is flanked by transposon inverted repeats and the transposon inverted repeats are flanked by recombinase direct repeats. In certain such embodiments, at least one function of a support vector or support genome can be to encode, express, and/or deliver to a target cell a recombinase for recombination of recombinase sites present in a nucleic acid payload administered to the target cell. In various embodiments, a support vector or support genome can encode, express, and/or deliver to a target cell a recombinase for recombination of recombinase sites present in a nucleic acid payload administered to the target cell and also encode, express, and/or deliver to a target cell a transposase for transposition of an integrating fragment present in a nucleic acid payload administered to the target cell.

Various methods and compositions provided herein provide stable integration of an integrating fragment into a target cell genome. Stable integration includes, for example, that the integrating fragment remains present in the target cell genome for more than a transient period of time, e.g., in that it is passed on a part of the chromosomal genetic material to progeny of the target cell.

### IV. Vectors

The present disclosure includes various vectors that can include, encode, and/or deliver to a target cell a nucleic acid payload of the present disclosure, e.g., a nucleic acid payload encoding (e.g., among other things) an editing system engineered to modify an endogenous EpoR nucleic acid to produce a modified EpoR nucleic acid that encodes signaling-enhanced EpoR. The present disclosure includes various vectors that can include, encode, and/or deliver to a target cell a support nucleic acid payload of the present disclosure. Vectors of the present disclosure include agents for delivery of a nucleic acid to a subject, cell, or system. In various embodiments, a nucleic acid payload of the present disclosure is delivered by a vector such as a nanoparticle, lipid nanoparticle, liposome, plasmid, cosmid, virus, or phage.

In various embodiments, a nucleic acid payload of the present disclosure is included in and/or associated with a nanoparticle. Nanoparticles (NPs) can range in size from 10 to 1000 nm. Various nanoparticles that can include nucleic acids are known in the art. Examples include noble metal NPs, nanorods (NRs), nanoclusters (NCs), semiconductor quantum dots (QDs), and carbon allotropes such as single-wall carbon nanotubes (SWCNTs) and graphene. Particular examples further include gold NPs, silver NPs, gold NRs, gold/silver hybrids, silver NCs, magnetic nanoparticles, platinum NPs, palladium NPs, graphene oxide, micelles, polyacrylamide NPs, viral NPs, ferritin NPs, upconversion NPs, chalcogenide NPs, alkaline earth metal NPs, and DNA NPs. The present disclosure further includes lipid nanoparticles (LNPs, e.g., solid lipid nanoparticles (SLNs) and nanostructured lipid carriers (NLCs)).

In various embodiments, a nucleic acid payload of the present disclosure is encapsulated in an LNP. LNPs can include cationic lipids together with other components such as neutral phospholipids, phosphatidylcholines, sterols such as cholesterol, and/or PEGylated phospholipids. SLNs, produced using lipids that are solid at room temperature and at body temperature, are colloidal nanoparticles with a solid lipophilic core. The solid lipid core of an SLN can include triglycerides (e.g., tri-stearin), glyceride mixtures or partial glycerides (e.g., Imwitor), fatty acids (e.g., stearic acid or palmitic acid), steroids (e.g., cholesterol), and/or waxes (e.g., cetyl palmitate) that are solid at both room temperature and human body temperature. Lipid nano-emulsions (LNE) are colloidal nanoparticles with a core that is liquid at room temperature. NLCs include a mixture of solid and liquid lipids, such as glyceryl tricaprylate, ethyl oleate, isopropyl myristate, and/or glyceryl dioleate.

In various embodiments, a nucleic acid payload of the present disclosure is encapsulated in a liposome. Liposomes can include one or more phospholipid bilayers. Phospholipids can be organized in a bilayer structure due to their amphipathic properties, forming vesicles. Phospholipids can include, for example, phosphatidyl choline (lecithin; PC), phosphatidyl ethanolamine (cephalin; PE), phosphatidyl serine (PS), phosphatidyl inositol (PI), and/or phosphatidyl glycerol (PG). Liposomes can further include additional agents such as cholesterol, lipid chains, and/or surfactants. In various embodiments, cholesterol does not form a bilayer by itself, but can incorporate into phospholipid membranes. In various embodiments, a liposome can include a hydrophilic carbohydrate or polymer, such as a lipid derivative of polyethylene glycol (PEG). Liposomes include conventional liposomes, pH sensitive liposomes, cationic liposomes, immune liposomes, and long circulating liposomes. Liposomes include multilamellar vesicles and unilamellar vesicles (e.g., large and small unilamellar vesicles).

In various embodiments, a nucleic acid payload of the present disclosure is present in a viral genome and/or encapsidated in a viral particle of a virus. Various types of viruses can be used for delivery of nucleic acids. Viruses for delivery of a nucleic acid payload of the present disclosure can be adenoviruses, adeno-associated viruses, alphaviruses, flaviviruses, herpes simplex viruses (HSV), measles viruses, rhabdoviruses, retroviruses, lentiviruses, Newcastle disease virus (NDV), poxviruses, and picornaviruses.

In certain embodiments, a nucleic acid payload of the present disclosure is present in an adenoviral genome and/or encapsidated in a viral particle of an adenovirus. Adenoviruses are large, icosahedral-shaped, non-enveloped viruses. Natural adenoviral capsids include three types of proteins: fiber, penton, and hexon. The hexon makes up the majority of the viral capsid, forming 20 triangular faces. A penton base is located at each of the 12 vertices of the capsid, and a fiber (also referred to as a knobbed fiber) protrudes from each penton base. Penton and fiber, and in particular the fiber knob, are of particular importance in receptor binding and internalization as they facilitate the attachment of the capsid to host cells. In various embodiments, the adenovirus is a helper-dependent adenovirus. Exemplary adenoviral vectors are described herein.

The present disclosure includes Ad3, 5, 7, 11, 14, 16, 21, 34, 35, 37, or 50 genomes. In various embodiments, an Ad3, 5, 7, 11, 14, 16, 21, 34, 35, 37, or 50 genome is a single-stranded or double-stranded DNA sequence that includes ITRs of an Ad3, 5, 7, 11, 14, 16, 21, 34, 35, 37, or 50 vector (e.g., a 5′ ITR according to SEQ ID NO: 210, 19, 37, 55, 73, 91, 109, 127, 145, 163, or 181 and a 3′ ITR according to SEQ ID NO: 211, 20, 38, 56, 74, 92, 110, 128, 146, 164, or 182), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is a single-stranded or double-stranded DNA sequence that includes a packaging sequence of an Ad3, 5, 7, 11, 14, 16, 21, 34, 35, 37, or 50 vector (e.g., a packaging sequence according to SEQ ID NO: 212, 21, 39, 57, 75, 93, 111, 129, 147, 165, or 183), or a packaging sequence having at least 75% sequence identity (e.g., at least 75%, 80%, 85%, 90%, 95%, 96%, 97%, 98%, 99%, or 100% identity) to the entirety or a portion thereof. In various embodiments, an Ad3, 5, 7, 11, 14, 16, 21, 34, 35, 37, or 50 genome is a single-stranded or double-stranded DNA sequence that includes a sequence with at least 75% sequence identity (e.g., at least 75%, 80%, 85%, 90%, 95%, 96%, 97%, 98%, 99%, or 100% identity) to all, a portion of, or a contiguous corresponding portion of, or a discontiguous corresponding portion of a reference Ad3, 5, 7, 11, 14, 16, 21, 34, 35, 37, or 50 genome (e.g., SEQ ID NO: 199, 200, 201, 202, 203, 204, 205, 206, 207, 208, or 209).

In various embodiments, an Ad3, 5, 7, 11, 14, 16, 21, 34, 35, 37, or 50 genome is any nucleotide sequence that includes at least ITRs of an Ad3, 5, 7, 11, 14, 16, 21, 34, 35, 37, or 50 vector (e.g., a 5′ ITR according to SEQ ID NO: 210, 19, 37, 55, 73, 91, 109, 127, 145, 163, or 181 and a 3′ ITR according to SEQ ID NO: 211, 20, 38, 56, 74, 92, 110, 128, 146, 164, or 182), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is an Ad3, 5, 7, 11, 14, 16, 21, 34, 35, 37, or 50 genome from which one or more nucleotides, coding sequences, and/or genes are completely or partially deleted as compared to a reference sequence. For example, in some embodiments, an Ad3, 5, 7, 11, 14, 16, 21, 34, 35, 37, or 50 genome can be a genome that does not include one or more of E1, E2, E3, and E4. In certain embodiments, an Ad3, 5, 7, 11, 14, 16, 21, 34, 35, 37, or 50 genome is a genome that does not include any coding sequences of an Ad3, 5, 7, 11, 14, 16, 21, 34, 35, 37, or 50 genome (e.g., a “gutless” vector that includes ITRs having at least 75% sequence identity to Ad3, 5, 7, 11, 14, 16, 21, 34, 35, 37, or 50 genome ITRs but includes none of the coding sequences present in a reference Ad3, 5, 7, 11, 14, 16, 21, 34, 35, 37, or 50 genome).

In various embodiments, an Ad3, 5, 7, 11, 14, 16, 21, 34, 35, 37, or 50 genome includes, does not include, or includes a deletion of, all or a portion of an E1 sequence according to SEQ ID NO: 213, 22, 40, 58, 76, 94, 112, 130, 148, 166, or 184, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E2 sequence according to SEQ ID NO: 5, 23, 41, 59, 77, 95, 113, 131, 149, 167, or 185, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E3 sequence according to SEQ ID NO: 6, 24, 42, 60, 78, 96, 114, 132, 150, 168, or 186, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, or does not include, a sequence that encodes a fiber, wherein the sequence has at least 75% sequence identity (e.g., at least 75%, 80%, 85%, 90%, 95%, 96%, 97%, 98%, 99%, or 100% identity) to SEQ ID NO: 7, 25, 43, 61, 79, 97, 115, 133, 151, 169, or 187.

In various embodiments, an Ad3, 5, 7, 11, 14, 16, 21, 34, 35, 37, or 50 genome includes, or does not include, a sequence that encodes a fiber shaft, wherein the sequence has at least 75% sequence identity (e.g., at least 75%, 80%, 85%, 90%, 95%, 96%, 97%, 98%, 99%, or 100% identity) to SEQ ID NO: 9, 27, 45, 63, 81, 99, 117, 135, 153, 171, or 189.

In various embodiments, an Ad3, 5, 7, 11, 14, 16, 21, 34, 35, 37, or 50 genome includes, or does not include, a sequence that encodes a fiber knob, wherein the sequence has at least 75% sequence identity (e.g., at least 75%, 80%, 85%, 90%, 95%, 96%, 97%, 98%, 99%, or 100% identity) to SEQ ID NO: 10, 28, 46, 64, 82, 100, 118, 136, 154, 172, or 190.

In various embodiments, an Ad3, 5, 7, 11, 14, 16, 21, 34, 35, 37, or 50 genome includes, or does not include, a sequence that encodes a fiber tail, wherein the sequence has at least 75% sequence identity (e.g., at least 75%, 80%, 85%, 90%, 95%, 96%, 97%, 98%, 99%, or 100% identity) to SEQ ID NO: 8, 26, 44, 62, 80, 98, 116, 134, 152, 170, or 188.

In various embodiments, an Ad3, 5, 7, 11, 14, 16, 21, 34, 35, 37, or 50 genome includes, or does not include, a sequence that encodes a penton, wherein the sequence has at least 75% sequence identity (e.g., at least 75%, 80%, 85%, 90%, 95%, 96%, 97%, 98%, 99%, or 100% identity) to SEQ ID NO: 11, 29, 47, 65, 83, 101, 119, 137, 155, 173, or 191.

In various embodiments, an Ad3, 5, 7, 11, 14, 16, 21, 34, 35, 37, or 50 genome includes, or does not include, a sequence that encodes a hexon, wherein the sequence has at least 75% sequence identity (e.g., at least 75%, 80%, 85%, 90%, 95%, 96%, 97%, 98%, 99%, or 100% identity) to SEQ ID NO: 12, 30, 48, 66, 84, 102, 120, 138, 156, 174, or 192.

The present disclosure includes Ad3, 5, 7, 11, 14, 16, 21, 34, 35, 37, or 50 vectors that include a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 13, 31, 49, 67, 85, 103, 121, 139, 157, 175, or 193).

The present disclosure includes Ad3, 5, 7, 11, 14, 16, 21, 34, 35, 37, or 50 vectors that include a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 18, 36, 54, 72, 90, 108, 126, 144, 162, 180, or 198, e.g., where the fiber tail is the portion of the fiber including all amino acids N-terminal to the fiber shaft).

The present disclosure includes Ad3, 5, 7, 11, 14, 16, 21, 34, 35, 37, or 50 vectors that include a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 14, 32, 50, 68, 86, 104, 122, 140, 158, 176, or 194).

The present disclosure includes Ad3, 5, 7, 11, 14, 16, 21, 34, 35, 37, or 50 vectors that include a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 15, 33, 51, 69, 87, 105, 123, 141, 159, 177, or 195).

The present disclosure includes Ad3, 5, 7, 11, 14, 16, 21, 34, 35, 37, or 50 vectors that include a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 16, 34, 52, 70, 88, 106, 124, 142, 160, 178, or 196).

The present disclosure includes Ad3, 5, 7, 11, 14, 16, 21, 34, 35, 37, or 50 vectors that include a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 17, 35, 53, 71, 89, 107, 125, 143, 161, 179, or 197).

In various embodiments, an Ad3, 5, 7, 11, 14, 16, 21, 34, 35, 37, or 50 vector is any adenoviral vector that includes at least a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 13, 31, 49, 67, 85, 103, 121, 139, 157, 175, or 193).

In various embodiments, an Ad3, 5, 7, 11, 14, 16, 21, 34, 35, 37, or 50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 18, 36, 54, 72, 90, 108, 126, 144, 162, 180, or 198, e.g., where the fiber tail is the portion of the fiber including all amino acids N-terminal to the fiber shaft).

In various embodiments, an Ad3, 5, 7, 11, 14, 16, 21, 34, 35, 37, or 50 vector is any adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 14, 32, 50, 68, 86, 104, 122, 140, 158, 176, or 194).

In various embodiments, an Ad3, 5, 7, 11, 14, 16, 21, 34, 35, 37, or 50 vector is any adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 15, 33, 51, 69, 87, 105, 123, 141, 159, 177, or 195).

In various embodiments, an Ad3, 5, 7, 11, 14, 16, 21, 34, 35, 37, or 50 vector is any adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 16, 34, 52, 70, 88, 106, 124, 142, 160, 178, or 196).

In various embodiments, an Ad3, 5, 7, 11, 14, 16, 21, 34, 35, 37, or 50 vector is any adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 17, 35, 53, 71, 89, 107, 125, 143, 161, 179, or 197).

Thus, an Ad3, 5, 7, 11, 14, 16, 21, 34, 35, 37, or 50 vector can be a chimeric adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob and at least one protein or portion thereof (such as a fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft and at least one protein or portion thereof (such as a fiber knob, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail and at least one protein or portion thereof (such as a fiber knob, fiber shaft, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton and at least one protein or portion thereof (such as a fiber knob, fiber shaft, fiber tail, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon and at least one protein or portion thereof (such as a fiber knob, fiber shaft, fiber tail, or penton) that has at least 75% sequence identity (e.g., at least 75%, 80%, 85%, 90%, 95%, 96%, 97%, 98%, 99%, or 100% identity) to a different adenoviral serotype.

Exemplary sequences of Ad3, 5, 7, 11, 14, 16, 21, 34, 35, 37, or 50 components (e.g., ITRs, packaging sequences, genes, and proteins) are provided in the following tables. Viral polypeptides include proteins that are components of viral vectors and portions or fragments thereof, including for example a fiber, fiber knob, fiber shaft, fiber tail, penton, or hexon.

In various embodiments, an Ad35 fiber knob of an Ad35 vector or chimeric Ad vector that includes an Ad35 fiber knob is a mutant Ad35 fiber knob. In particular embodiments, a mutant Ad35 fiber knob is an Ad35++ mutant fiber knob (alternatively referred to herein as an Ad35++ fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593 (24): 3623-3648, 2019). In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession no. AP_000601 or an amino acid sequence corresponding thereto, e.g., where position 207 is Glu or Asp. In various embodiments, an Ad35 fiber has an amino acid sequence according to GenBank accession no. AP_000601. Further description of Ad35++ fiber knob mutations is found in Wang 2008 J. Virol. 82 (21): 10567-10579, which is incorporated herein by reference in its entirety and with respect to fiber knobs. The present disclosure includes, for example, a recombinant Ad35 vector with a mutant Ad35 fiber knob or an Ad5/35 vector with a mutant Ad35 fiber knob.

In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

Various sequences corresponding to accession numbers disclosed herein, including e.g., accession numbers referred to herein as SEQ ID NOs: 199, 200, 201, 202, 203, 204, 205, 206, 207, 208, and/or 209 as indicated in Tables 2-23, are provided herein in the below listing of accession sequences. Those of skill in the art will appreciate that such sequences, including the sequences disclosed in the below listing of accession sequences, can be referenced in whole (e.g., by an accession number) or in part (e.g., by reference to a nucleotide position and/or a set or range of nucleotide positions of a sequence and/or accession number).

TABLE 2
Ad3 Genomic Sequences
Reference Ad3 Genome Sequence: GenBank
accession no. NC_011203 (SEQ ID NO: 199)

| | Exemplary Sequence | |
|---|---|---|
| Component | (position in reference) | SEQ ID NO: |
| Ad3 5′ (left) ITR | 1-136 | 210 |
| Ad3 3′ (right) ITR | 35208-35343 | 211 |
| Ad3 Packaging | 137-479 | 212 |
| Sequence | | |
| Ad3 E1 | 480-3918 | 213 |
| Ad3 E2 | 26643-3947 | 5 |
| Ad3 E3 | 27085-31186 | 6 |
| Ad3 fiber | 31368-32327 | 7 |
| Ad3 fiber tail | 31368-31493 | 8 |
| Ad3 fiber shaft | 31494-31763 | 9 |
| Ad3 fiber knob | 31764-32324 | 10 |
| Ad3 penton | 13905-15539 | 11 |
| Ad3 hexon | 18418-21252 | 12 |

TABLE 3
Ad3 Amino Acid Sequences

| | Exemplary Sequence | |
|---|---|---|
| Component | (position in reference) | SEQ ID NO: |
| Ad3 fiber | 1-319 (GenBank accession | 13 |
| | no. YP_002213796) | |
| Ad3 fiber | 43-132 (GenBank accession | 14 |
| shaft | no. YP_002213796) | |
| Ad3 fiber | 134-319 (GenBank accession | 15 |
| knob | no. YP_002213796) | |
| Ad3 penton | 1-544 (GenBank accession | 16 |
| | no. YP_002213774) | |
| Ad3 hexon | 1-944 (GenBank accession | 17 |
| | no. YP_002213779) | |
| Ad3 fiber | 1-42 (GenBank accession | 18 |
| tail | no. YP_002213796) | |

TABLE 4
Ad7 Genomic Sequences
Reference Ad7 Genome Sequence: GenBank
accession no. AC_000018 (SEQ ID NO: 200)

| | Exemplary Sequence | |
|---|---|---|
| Component | (position in reference) | SEQ ID NO: |
| Ad7 5′ (left) ITR | 1-136 | 19 |
| Ad7 3′ (right) ITR | 35379-35514 | 20 |
| Ad7 Packaging | 137-479 | 21 |
| Sequence | | |
| Ad7 E1 | 480-3919 | 22 |
| Ad7 E2 | 26867-3947 | 23 |
| Ad7 E3 | 27308-31345 | 24 |
| Ad7 fiber | 31529-32506 | 25 |
| Ad7 fiber tail | 31529-31654 | 26 |
| Ad7 fiber shaft | 31655-31927 | 27 |
| Ad7 fiber knob | 31928-32503 | 28 |
| Ad7 penton | 14153-15787 | 29 |
| Ad7 hexon | 18666-21470 | 30 |

TABLE 5
Ad7 Amino Acid Sequences

| | Exemplary Sequence | |
|---|---|---|
| Component | (position in reference) | SEQ ID NO: |
| Ad7 fiber | 1-325 (GenBank accession | 31 |
| | no. AP_000564) | |
| Ad7 fiber | 43-133 (GenBank accession | 32 |
| shaft | no. AP_000564) | |
| Ad7 fiber | 134-325 (GenBank accession | 33 |
| knob | no. AP_000564) | |
| Ad7 penton | 1-544 (GenBank accession | 34 |
| | no. AP_000543) | |
| Ad7 hexon | 1-934 (GenBank accession | 35 |
| | no. AP_000548) | |
| Ad7 fiber | 1-42 (GenBank accession | 36 |
| tail | no. AP_000564) | |

TABLE 6
Ad11 Genomic Sequences
Reference Ad11 Genome Sequence: GenBank
accession no. NC_011202 (SEQ ID NO: 201)

| | Exemplary Sequence | |
|---|---|---|
| Component | (position in reference) | SEQ ID NO: |
| Ad11 5′ (left) ITR | 1-137 | 37 |
| Ad11 3′ (right) ITR | 34658-34794 | 38 |
| Ad11 Packaging | 138-479 | 39 |
| Sequence | | |
| Ad11 E1 | 480-3931 | 40 |
| Ad11 E2 | 25445-3963 | 41 |
| Ad11 E3 | 26866-30624 | 42 |
| Ad11 fiber | 30811-31788 | 43 |
| Ad11 fiber tail | 30811-30936 | 44 |
| Ad11 fiber shaft | 30937-31209 | 45 |
| Ad11 fiber knob | 31210-31785 | 46 |
| Ad11 penton | 13682-15367 | 47 |
| Ad11 hexon | 18254-21100 | 48 |

TABLE 7
Ad11 Amino Acid Sequences

| | Exemplary Sequence | |
|---|---|---|
| Component | (position in reference) | SEQ ID NO: |
| Ad11 fiber | 1-325 (GenBank accession | 49 |
| | no. YP_002213828) | |
| Ad11 fiber | 43-133 (GenBank accession | 50 |
| shaft | no. YP_002213828) | |
| Ad11 fiber | 134-325 (GenBank accession | 51 |
| knob | no. YP_002213828) | |
| Ad11 penton | 1-561 (GenBank accession | 52 |
| | no. YP_002213807) | |
| Ad11 hexon | 1-948 (GenBank accession | 53 |
| | no. YP_002213812) | |
| Ad11 fiber | 1-42 (GenBank accession | 54 |
| tail | no. YP_002213828) | |

TABLE 8
Ad14 Genomic Sequences
Reference Ad14 Genome Sequence: GenBank
accession no. AY803294 (SEQ ID NO: 202)

| | Exemplary Sequence | |
|---|---|---|
| Component | (position in reference) | SEQ ID NO: |
| Ad14 5′ (left) ITR | 1-137 | 55 |
| Ad14 3′ (right) ITR | 34628-34764 | 56 |
| Ad14 Packaging | 138-479 | 57 |
| Sequence | | |
| Ad14 E1 | 480-3947 | 58 |
| Ad14 E2 | 23389-3963 | 59 |
| Ad14 E3 | 26854-30601 | 60 |
| Ad14 fiber | 30788-31765 | 61 |
| Ad14 fiber tail | 30788-30913 | 62 |
| Ad14 fiber shaft | 30914-31186 | 63 |
| Ad14 fiber knob | 31187-31762 | 64 |
| Ad14 penton | 13698-15374 | 65 |
| Ad14 hexon | 18252-21089 | 66 |

TABLE 9
Ad14 Amino Acid Sequences
Ad14 Amino Acid Sequences

| | Exemplary Sequence | SEQ |
|---|---|---|
| Component | (position in reference) | ID NO: |
| Ad14 fiber | 1-325 (GenBank accession no. AAW33140) | 67 |
| Ad14 fiber shaft | 43-133 (GenBank accession no. AAW33140) | 68 |
| Ad14 fiber knob | 134-325 (GenBank accession no. AAW33140) | 69 |
| Ad14 penton | 1-558 (GenBank accession no. AAW33119) | 70 |
| Ad14 hexon | 1-945 (GenBank accession no. AAW33124) | 71 |
| Ad14 fiber tail | 1-42 (GenBank accession no. AAW33140) | 72 |

TABLE 10
Ad16 Genomic Sequences
Ad16 Genomic Sequences
Reference Ad16 Genome Sequence: GenBank accession no. AY601636
(SEQ ID NO: 203)

| | Exemplary Sequence | SEQ |
|---|---|---|
| Component | (position in reference) | ID NO: |
| Ad16 5′ (left) ITR | 1-114 | 73 |
| Ad16 3′ (right) ITR | 35409-35522 | 74 |
| Ad16 Packaging | 115-479 | 75 |
| Sequence | | |
| Ad16 E1 | 480-3910 | 76 |
| Ad16 E2 | 23580-3954 | 77 |
| Ad16 E3 | 27107-31263 | 78 |
| Ad16 fiber | 31448-32509 | 79 |
| Ad16 fiber tail | 31448-31573 | 80 |
| Ad16 fiber shaft | 31574-31933 | 81 |
| Ad16 fiber knob | 31934-32506 | 82 |
| Ad16 penton | 13902-17534 | 83 |
| Ad16 hexon | 18450-21272 | 84 |

TABLE 11
Ad16 Amino Acid Sequences
Ad16 Amino Acid Sequences

| | Exemplary Sequence | SEQ |
|---|---|---|
| Component | (position in reference) | ID NO: |
| Ad16 fiber | 1-353 (GenBank accession no. AAW33461) | 85 |
| Ad16 fiber shaft | 43-172 (GenBank accession no. AAW33461) | 86 |
| Ad16 fiber knob | 173-353 (GenBank accession no. AAW33461) | 87 |
| Ad16 penton | 1-555 (GenBank accession no. AAW33439) | 88 |
| Ad16 hexon | 1-940 (GenBank accession no. AAW33444) | 89 |
| Ad16 fiber tail | 1-42 (GenBank accession no. AAW33461) | 90 |

TABLE 12
Ad21 Genomic Sequences
Ad21 Genomic Sequences
Reference Ad21 Genome Sequence: GenBank accession no. AY601633
(SEQ ID NO: 204)

| | Exemplary Sequence | SEQ |
|---|---|---|
| Component | (position in reference) | ID NO: |
| Ad21 5′ (left) ITR | 1-114 | 91 |
| Ad21 3′ (right) ITR | 35269-35382 | 92 |
| Ad21 Packaging | 115-479 | 93 |
| Sequence | | |
| Ad21 E1 | 480-3911 | 94 |
| Ad21 E2 | 23611-3924 | 95 |
| Ad21 E3 | 27441-31208 | 96 |
| Ad21 fiber | 31406-32377 | 97 |
| Ad21 fiber tail | 31406-31531 | 98 |
| Ad21 fiber shaft | 31532-31804 | 99 |
| Ad21 fiber knob | 31805-32374 | 100 |
| Ad21 penton | 13878-15563 | 101 |
| Ad21 hexon | 18454-21303 | 102 |

TABLE 13
Ad21 Amino Acid Sequences
Ad21 Amino Acid Sequences

| | Exemplary Sequence | SEQ |
|---|---|---|
| Component | (position in reference) | ID NO: |
| Ad21 fiber | 1-323 (GenBank accession no. AAW33370) | 103 |
| Ad21 fiber shaft | 43-133 (GenBank accession no. AAW33370) | 104 |
| Ad21 fiber knob | 134-323 (GenBank accession no. AAW33370) | 105 |
| Ad21 penton | 1-561 (GenBank accession no. AAW33349) | 106 |
| Ad21 hexon | 1-949 (GenBank accession no. AAW33354) | 107 |
| Ad21 fiber tail | 1-42 (GenBank accession no. AAW33370) | 108 |

TABLE 14
Ad34 Genomic Sequences
Ad34 Genomic Sequences
Reference Ad34 Genome Sequence: GenBank accession no. AY737797
(SEQ ID NO: 205)

| | Exemplary Sequence | SEQ |
|---|---|---|
| Component | (position in reference) | ID NO: |
| Ad34 5′ (left) ITR | 1-137 | 109 |
| Ad34 3′ (right) ITR | 34639-34775 | 110 |
| Ad34 Packaging | 138-479 | 111 |
| Sequence | | |
| Ad34 E1 | 480-3929 | 112 |
| Ad34 E2 | 23399-3945 | 113 |
| Ad34 E3 | 27185-30625 | 114 |
| Ad34 fiber | 30812-31783 | 115 |
| Ad34 fiber tail | 30812-30937 | 116 |
| Ad34 fiber shaft | 30938-31210 | 117 |
| Ad34 fiber knob | 31211-31780 | 118 |
| Ad34 penton | 13681-15357 | 119 |
| Ad34 hexon | 18244-21099 | 120 |

TABLE 15
Ad34 Amino Acid Sequences
Ad34 Amino Acid Sequences

| | | | SEQ |
|---|---|---|---|
| | Exemplary Sequence | | ID |
| Component | (position in reference) | | NO: |
| Ad34 fiber | 1-323 | (GenBank accession no. AAW33501) | 121 |
| Ad34 fiber shaft | 43-133 | (GenBank accession no. AAW33501) | 122 |
| Ad34 fiber knob | 134-323 | (GenBank accession no. AAW33501) | 123 |
| Ad34 penton | 1-558 | (GenBank accession no. ABC49791) | 124 |
| Ad34 hexon | 1-951 | (GenBank accession no. AAW33485) | 125 |
| Ad34 fiber tail | 1-42 | (GenBank accession no. AAW33501) | 126 |

TABLE 16
Ad37 Genomic Sequences
Ad37 Genomic Sequences
Reference Ad37 Genome Sequence: GenBank accession no. DQ900900
(SEQ ID NO: 206)

| | Exemplary Sequence | SEQ |
|---|---|---|
| Component | (position in reference) | ID NO: |
| Ad37 5′ (left) ITR | 1-159 | 127 |
| Ad37 3′ (right) ITR | 35055-35213 | 128 |
| Ad37 Packaging | 160-479 | 129 |
| Sequence | | |
| Ad37 E1 | 480-3867 | 130 |
| Ad37 E2 | 22777-3902 | 131 |
| Ad37 E3 | 26198-30771 | 132 |
| Ad37 fiber | 31038-32135 | 133 |
| Ad37 fiber tail | 31038-31163 | 134 |
| Ad37 fiber shaft | 31164-31592 | 135 |
| Ad37 fiber knob | 31593-32132 | 136 |
| Ad37 penton | 13530-15089 | 137 |
| Ad37 hexon | 17775-20624 | 138 |

TABLE 17
Ad37 Amino Acid Sequences
Ad37 Amino Acid Sequences

| | Exemplary Sequence | SEQ |
|---|---|---|
| Component | (position in reference) | ID NO: |
| Ad37 fiber | 1-361 (GenBank accession no. ABK59080) | 139 |
| Ad37 fiber shaft | 43-185 (GenBank accession no. ABK59080) | 140 |
| Ad37 fiber knob | 186-361 (GenBank accession no. ABK59080) | 141 |
| Ad37 penton | 1-519 (GenBank accession no. ABK59086) | 142 |
| Ad37 hexon | 1-949 (GenBank accession no. ABK59070) | 143 |
| Ad37 fiber tail | 1-42 (GenBank accession no. ABK59080) | 144 |

TABLE 18
Ad50 Genomic Sequences
Ad50 Genomic Sequences
Reference Ad50 Genome Sequence: GenBank accession no. AY737798
(SEQ ID NO: 207)

| | Exemplary Sequence | SEQ |
|---|---|---|
| Component | (position in reference) | ID NO: |
| Ad50 5′ (left) ITR | 1-114 | 145 |
| Ad50 3′ (right) ITR | 35272-35385 | 146 |
| Ad50 Packaging | 115-479 | 147 |
| Sequence | | |
| Ad50 E1 | 480-3910 | 148 |
| Ad50 E2 | 23590-3923 | 149 |
| Ad50 E3 | 27102-31222 | 150 |
| Ad50 fiber | 31409-32380 | 151 |
| Ad50 fiber tail | 31409-31534 | 152 |
| Ad50 fiber shaft | 31535-31807 | 153 |
| Ad50 fiber knob | 31808-32377 | 154 |
| Ad50 penton | 13888-15570 | 155 |
| Ad50 hexon | 18460-21282 | 156 |

TABLE 19
Ad50 Amino Acid Sequences
Ad50 Amino Acid Sequences

| | Exemplary Sequence | SEQ |
|---|---|---|
| Component | (position in reference) | ID NO: |
| Ad50 fiber | 1-323 (GenBank accession no. AAW33547) | 157 |
| Ad50 fiber shaft | 43-133 (GenBank accession no. AAW33547) | 158 |
| Ad50 fiber knob | 134-323 (GenBank accession no. AAW33547) | 159 |
| Ad50 penton | 1-560 (GenBank accession no. AAW33525) | 160 |
| Ad50 hexon | 1-940 (GenBank accession no. AAW33530) | 161 |
| Ad50 fiber tail | 1-42 (GenBank accession no. AAW33547) | 162 |

TABLE 20
Ad5 Genomic Sequences
Ad5 Genomic Sequences
Reference Ad5 Genome Sequence: GenBank accession no. AC 000008
(SEQ ID NO: 208)

| | Exemplary Sequence | SEQ |
|---|---|---|
| Component | (position in reference) | ID NO: |
| Ad5 5′ (left) ITR | 1-103 | 163 |
| Ad5 3′ (right) ITR | 35836-35938 | 164 |
| Ad5 Packaging | 104-479 | 165 |
| Sequence | | |
| Ad5 E1 | 480-3509 | 166 |
| Ad5 E2 | 4091-24032 | 167 |
| Ad5 E3 | 27174-30839 | 168 |
| Ad5 fiber | 31042-32787 | 169 |
| Ad5 fiber tail | 31042-31170 | 170 |
| Ad5 fiber shaft | 31171-32241 | 171 |
| Ad5 fiber knob | 32242-32784 | 172 |
| Ad5 penton | 14156-15871 | 173 |
| Ad5 hexon | 18842-21700 | 174 |

TABLE 21
Ad5 Amino Acid Sequences
Ad5 Amino Acid Sequences

| | | | SEQ |
|---|---|---|---|
| | Exemplary Sequence | | ID |
| Component | (position in reference) | | NO: |
| Ad5 fiber | 1-581 | (GenBank accession no. AP_000226) | 175 |
| Ad5 fiber shaft | 44-400 | (GenBank accession n. AP_000226) | 176 |
| Ad5 fiber knob | 401-581 | (GenBank accession no. AP_000226) | 177 |
| Ad5 penton | 1-571 | (GenBank accession no. AP_000206) | 178 |
| Ad5 hexon | 1-952 | (GenBank accession no. AP_000211) | 179 |
| Ad5 fiber tail | 1-43 | (GenBank accession no. AP_000226) | 180 |

TABLE 22
Ad35 Genomic Sequences
Ad35 Genomic Sequences
Reference Ad5 Genome Sequence: GenBank accession no. AY128640
(SEQ ID NO: 209)

| | Exemplary Sequence | SEQ |
|---|---|---|
| Component | (position in reference) | ID NO: |
| Ad35 5′ (left) ITR | 1-137 | 181 |
| Ad35 3′ (right) ITR | 34658-34794 | 182 |
| Ad35 Packaging | 138-479 | 183 |
| Sequence | | |
| Ad35 E1 | 480-3400 | 184 |
| Ad35 E2 | 3966-23415 | 185 |
| Ad35 E3 | 27198-30622 | 186 |
| Ad35 fiber | 30826-31797 | 187 |
| Ad35 fiber tail | 30826-30951 | 188 |
| Ad35 fiber shaft | 30952-31224 | 189 |
| Ad35 fiber knob | 31225-31797 | 190 |
| Ad35 penton | 13690-15375 | 191 |
| Ad35 hexon | 18255-21113 | 192 |

TABLE 23
Ad35 Amino Acid Sequences
Ad35 Amino Acid Sequences

| | Exemplary Sequence | SEQ |
|---|---|---|
| Component | (position in reference) | ID NO: |
| Ad35 fiber | 1-323 (GenBank accession no. AP_000601) | 193 |
| Ad35 fiber shaft | 43-133 (GenBank accession no. AP_000601) | 194 |
| Ad35 fiber knob | 134-323 (GenBank accession no. AP_000601) | 195 |
| Ad35 penton | 1-561 (GenBank accession no. AP_000580) | 196 |
| Ad35 hexon | 1-952 (GenBank accession no. AP_000585) | 197 |
| Ad35 fiber tail | 1-42 (GenBank accession no. AP_000601) | 198 |

In various embodiments, an adenoviral vector or genome can be a helper dependent adenoviral vector (“HDAd” vector or genome). In various embodiments, an adenoviral vector or genome includes modifications that render viral replication dependent upon polypeptides that are not encoded by the viral genome, which can instead by provided by a “helper.” Helper dependency can reduce and/or eliminate replication of the virus in recipients (e.g., in the absence of a helper vector or helper vector genome). Broadly, there are three recognized “generations” of adenoviral vectors and genomes engineered to reduce and/or eliminate replication of the virus in recipients. Adenoviral vectors of the present disclosure can include vectors according to any of these three generations.

In various embodiments, an Adenoviral genome differs from a reference Ad sequence (e.g., one or more canonical, representative, exemplary, or wild-type sequence of an adenovirus of a serotype of interest) at least in that the regulatory E1 gene (E1a and E1b) is removed from the Ad genome (“first generation” vector modifications). E1a and E1b are the first transcriptional regulatory factors produced during the adenoviral replication cycle. E1 deletion reduces or eliminates expression of certain viral genes controlled by E1, and E1-deleted helper viruses are replication-defective. Thus, first generation adenoviral vectors are deficient for replication in a recipient. In some embodiments, first-generation adenoviral vectors are engineered to remove E1 and E3 genes. Retained portions of the reference genome can be identical in sequence to a reference genome or can have less than 100% identity with a reference genome, e.g., at least 99%, 98%, 97%, 96%, 95%, 90%, 85%, 80%, or 75% identity. Without these E1 (or E1 and E3) genes, adenoviral vectors cannot replicate on their own but can be produced in mammalian cell lines that express E1 (e.g., of the same serotype) or another protein sufficient to restore expression of the certain viral genes. For illustration, where an E1-deficient Ad5 vector encodes an Ad5 E4orf6, the helper vector can be propagated in a cell line that expresses Ad5 E1. In one exemplary cell type for adenoviral vector production, HEK293 cells express Ad5 E1b55k, which is known to form a complex with Ad5 E4 protein ORF6.

In various embodiments, an adenoviral genome differs from a reference Ad sequence at least in that the E1 gene (E1a and E1b) and one or more of non-structural genes E2, E3 and/or E4 are deleted (“second generation” modifications). Second generation Ads have greater payload capacity than first generation Ads and are more deficient for replication than first generation viruses. In some embodiments, second-generation adenoviral vectors, in addition to E1/E3 removal, are engineered to remove non-structural genes E2 and E4, resulting in increased capacity and reduced immunogenicity. Retained portions of the reference genome can be identical in sequence to a reference genome or can have less than 100% identity with a reference genome, e.g., at least 99%, 98%, 97%, 96%, 95%, 90%, 85%, 80%, or 75% identity.

In various embodiments, an adenoviral genome differs from a reference Ad sequence at least in that it is engineered to remove all viral coding sequences from the Ad genome, and retain only the ITRs of the genome and the packaging sequence of the genome or a functional fragment thereof (“third generation” modifications). Third generation adenoviral vectors can also be referred to as gutless, high capacity adenoviral vectors, or helper-dependent adenoviral vectors (HdAds). Retained portions of the reference genome can be identical in sequence to a reference genome or can have less than 100% identity with a reference genome, e.g., at least 99%, 98%, 97%, 96%, 95%, 90%, 85%, 80%, or 75% identity.

Because third generation Ad genomes do not encode the proteins necessary for viral production, they are helper-dependent: a helper-dependent genome can only be packaged into a vector if they are present in a cell that includes a nucleic acid sequence that provides viral proteins in trans. These helper-dependent vectors are also characterized by still greater capacity than first and second generation vectors and decreased immunogenicity. Because HDAd vectors do not express viral genes when used as a vector, the risk of cytotoxicity or interferon response in recipients is reduced.

Helper-dependent adenoviral vectors engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and packaging (v), cellular immune response against the Ad vector is reduced. HDAd vectors have a large cloning capacity of up to about 37 kb, allowing for the delivery of large payloads.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes all of the proteins (e.g., all of the structural viral proteins) required for replication but has a conditional defect in the packaging sequence, making it less likely to be packaged into a vector under certain vector production conditions (e.g., in the presence of an agent that reduces function of the conditionally defective packaging sequence). Thus, the HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor viral genome to be selectively packaged into HDAd viral vectors produced from structural components expressed from the helper vector genome. In other words, Adenoviral helper vectors can be used for production of Adenoviral donor vectors. Production of HD Adenoviral vectors can include co-transfection of a plasmid containing the HDAd vector genome and a packaging-defective helper virus that provides structural and non-structural viral proteins. The helper virus genome can rescue propagation of the Adenoviral donor vector and Adenoviral donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, NJ: 2009. pp. 33-53. In some embodiments, a helper genome is E1-deficient.

In some HDAd vector systems, a helper genome utilizes a recombinase system (e.g., a Cre/loxP system) for conditional packaging. In certain such HDAd vector systems, a helper genome can include a packaging sequence or functional fragment thereof (e.g., a fragment of the packaging sequence that is sufficient for packaging, required for packaging, or required for efficient packaging of the Ad genome into the capsid) flanked by recombinase (e.g., loxP) sites so that contact with a corresponding recombinase (e.g., Cre recombinase) excises the packaging sequence or functional fragment thereof from the helper genome by recombinase-mediated (e.g., Cre-mediated) site-specific recombination between the recombinase sites (e.g., loxP sites). The present disclosure includes, among other things, Adenoviral helper vectors and genomes that include two recombination sites that flank a packaging sequence or functional fragment thereof, where the two recombination sites are sites corresponding to (i.e., for, or acted upon by) the same recombinase.

In various embodiments, a helper genome can include deletion of E1, e.g., where the helper genome includes all of the viral genes except for E1, as E1 expression products can be supplied by complementary expression from the genome of a producer cell line. In some embodiments, to prevent generation of replication competent Ad (RCA) as a consequence of homologous recombination between the helper and HDAd donor genomes present in producer cells, a “stuffer” sequence can be inserted into the E3 region to render any recombinants too large to be packaged and/or efficiently packaged.

For production of HDAd vectors, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence or functional fragment thereof flanked by recombinase sites (e.g., loxP sites). Thus, producer cells can be transfected with the HDAd donor genome and transduced with a helper genome bearing a packaging sequence or a functional fragment thereof flanked by recombinase sites (e.g., loxP sites), where the cells express a recombinase (e.g., Cre) corresponding to the recombinase sites such that excision of the packaging sequence or functional fragment thereof renders the helper virus genome deficient for packaging (e.g., unpackageable), but still able to provide all of the necessary trans-acting factors for production of HDAd donor vector including the HDAd donor genome.

Similar HDAd production systems have been developed using FLP (e.g., FLPe)/frt site-specific recombination, where FLP-mediated recombination between frt sites flanking the packaging sequence or functional fragment thereof of the helper genome reduces or eliminates packaging of helper genomes in producer cells that express FLP.

HDAd vectors including the donor vector genome including the payload can be isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

HDAd3, 5, 7, 11, 14, 16, 21, 34, 35, 37, and 50 donor vectors, donor genomes, helper vectors, and helper genomes are also exemplary of compositions provided herein and can be used in various methods of the present disclosure. An HDAd3, 5, 7, 11, 14, 16, 21, 34, 35, 37, or 50 vector or genome is a helper-dependent Ad3, 5, 7, 11, 14, 16, 21, 34, 35, 37, or 50 vector or genome. An Ad3, 5, 7, 11, 14, 16, 21, 34, 35, 37, or 50 helper vector is a vector that includes a helper genome that includes a conditionally expressed (e.g., frt-site or loxP-site flanked) packaging sequence or fragment thereof and encodes all of the necessary trans-acting factors for production of Ad3, 5, 7, 11, 14, 16, 21, 34, 35, 37, or 50 virions into which the donor genome can be packaged.

The present disclosure further includes an HDAd3, 5, 7, 11, 14, 16, 21, 34, 35, 37, or 50 donor vector production system including a cell including an HDAd3, 5, 7, 11, 14, 16, 21, 34, 35, 37, or 50 donor genome and an Ad3, 5, 7, 11, 14, 16, 21, 34, 35, 37, or 50 helper genome.

In certain such cells, viral proteins encoded and expressed by the helper genome can be utilized in production of HDAd3, 5, 7, 11, 14, 16, 21, 34, 35, 37, or 50 donor vectors in which the HDAd3, 5, 7, 11, 14, 16, 21, 34, 35, 37, or 50 donor genome is packaged. Accordingly, the present disclosure includes methods of production of HDAd3, 5, 7, 11, 14, 16, 21, 34, 35, 37, or 50 donor vectors by culturing cells that include an HDAd3, 5, 7, 11, 14, 16, 21, 34, 35, 37, or 50 donor genome and an Ad3, 5, 7, 11, 14, 16, 21, 34, 35, 37, or 50 helper genome. In some embodiments, the cells encode and express a recombinase that corresponds to recombinase direct repeats that flank a packaging sequence of the Ad3, 5, 7, 11, 14, 16, 21, 34, 35, 37, or 50 helper vector. In some embodiments, the flanked packaging sequence of the Ad3, 5, 7, 11, 14, 16, 21, 34, 35, 37, or 50 helper genome has been excised.

In some embodiments, the Ad3, 5, 7, 11, 14, 16, 21, 34, 35, 37, or 50 helper genome encodes all Ad3, 5, 7, 11, 14, 16, 21, 34, 35, 37, or 50 coding sequences. In some embodiments, the Ad3, 5, 7, 11, 14, 16, 21, 34, 35, 37, or 50 helper genome encodes and/or expresses all Ad3, 5, 7, 11, 14, 16, 21, 34, 35, 37, or 50 coding sequences except for one or more coding sequences of E1 and/or an E3 coding sequence and/or an E4 coding sequence. In various embodiments, a helper genome that does not encode and/or express an Ad3, 5, 7, 11, 14, 16, 21, 34, 35, 37, or 50 E1 gene does not encode and/or express an Ad3, 5, 7, 11, 14, 16, 21, 34, 35, 37, or 50 E4 gene. In various embodiments, as will be appreciate by those of skill in the art, cells of compositions and methods for production of HDAd donor vectors can be cells that express an E1 expression product.

The present disclosure includes, among other things, HDAd3, 5, 7, 11, 14, 16, 21, 34, 35, 37, or 50 donor vectors and genomes that include Ad3, 5, 7, 11, 14, 16, 21, 34, 35, 37, or 50 ITRs (a 5′ Ad3, 5, 7, 11, 14, 16, 21, 34, 35, 37, or 50 ITR and a 3′ ITR of the same serotype), e.g., where two Ad3, 5, 7, 11, 14, 16, 21, 34, 35, 37, or 50 ITRs flank a packaging sequence and a payload. The present disclosure includes, among other things, HDAd3, 5, 7, 11, 14, 16, 21, 34, 35, 37, or 50 donor vectors and genomes in which E1 or a fragment thereof is deleted. The present disclosure includes, among other things, HDAd3, 5, 7, 11, 14, 16, 21, 34, 35, 37, or 50 vectors and genomes in which E3 or a fragment thereof is deleted.

In various embodiments, excision of a packaging sequence or functional fragment thereof from an Ad3, 5, 7, 11, 14, 16, 21, 34, 35, 37, or 50 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9% or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type Ad3, 5, 7, 11, 14, 16, 21, 34, 35, 37, or 50 vector under comparable conditions.

An additional optional engineering consideration can be engineering of a helper genome having a size that permits separation of helper vector from HDAd3, 5, 7, 11, 14, 16, 21, 34, 35, 37, or 50 donor vector by centrifugation, e.g., by CsCl ultracentrifugation. One means of achieving this result is to increase the size of the helper genome as compared to a typical Ad3, 5, 7, 11, 14, 16, 21, 34, 35, 37, or 50 genome. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure can include a selection of components each selected from, or having at least 75% sequence identity (e.g., at least 75%, 80%, 85%, 90%, 95%, 96%, 97%, 98%, 99%, or 100% identity) to, a corresponding sequence of a single particular serotype. To provide an illustrative example, all components can correspond to (e.g., have at least 75% sequence identity to sequences of) Ad34, excepting sequences otherwise indicated (e.g., a payload, e.g., a heterologous payload).

It has also been observed that the certain HDAd vector genomes can be most efficiently packaged when the genome has at least a minimum a total length, e.g., a minimum to total length of at least 20 kb (e.g., 20, 21, 22, 23, 24, 25, 26, 27, 28, 29, 30, 31, 32, 33, 34, or 35 kb) which length can include, e.g., a therapeutic payload and/or a “stuffer” sequence. Where a payload does not utilize a number of nucleotides that causes the adenoviral genome to have at least a target length, a stuffer sequence can be used to achieve or surpass the target length. The present disclosure includes that a minimum length for efficient packaging is not required for beneficial use of vectors provided herein, such that meeting any target length may be advantageous but not required for use of compositions and methods provided herein.

In particular embodiments, the vector includes a stuffer sequence. In particular embodiments, the stuffer sequence may be added to render the genome at a size near that of wild-type length. Stuffer is a term generally recognized in the art intended to define functionally inert sequence intended to extend the length of the genome. The stuffer sequence is used to achieve efficient packaging and stability of the vector. In particular embodiments, the stuffer sequence is used to render the genome size between 70% and 110% of that of the wild type virus. The stuffer sequences can be any DNA, preferably of mammalian origin. In a preferred embodiment of the invention, stuffer sequences are non-coding sequences of mammalian origin, for example intronic fragments. The stuffer sequence, when used to keep the size of the vector a predetermined size, can be any non-coding sequence or sequence that allows the genome to remain stable in dividing or nondividing cells. These sequences can be derived from other viral genomes (e.g., Epstein bar virus) or organism (e.g., yeast). For example, these sequences could be a functional part of centromeres and/or telomeres.

In various embodiments, and adenovirus of the present disclosure is an Ad35, Ad5/35, or Ad5 adenovirus. In various embodiments, the present disclosure includes Ad35 genomes, Ad35 vectors, Ad5/35 genomes, Ad5/35 vectors, Ad5 genomes, and Ad5 vectors. In various embodiments, and adenovirus of the present disclosure is an HDAd35, HDAd5/35, or HDAd5 adenovirus. In various embodiments, the present disclosure includes HDAd35 genomes, HDAd35 vectors, HDAd5/35 genomes, HDAd5/35 vectors, Ad5 genomes, and HDAd5 vectors. In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes (e.g., Ad35 and Ad5/35 vectors and genomes).

In various embodiments, an Ad35 fiber knob of an Ad35 vector or chimeric Ad vector that includes an Ad35 fiber knob is a mutant Ad35 fiber knob. In particular embodiments, a mutant Ad35 fiber knob is an Ad35++ mutant fiber knob (alternatively referred to herein as an Ad35++ fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593 (24): 3623-3648, 2019). In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession no. AP_000601 or an amino acid sequence corresponding thereto, e.g., where position 207 is Glu or Asp. In various embodiments, an Ad35 reference fiber has an amino acid sequence according to GenBank accession no. AP_000601. Further description of Ad35++ fiber knob mutations is found in Wang 2008 J. Virol. 82 (21): 10567-10579, which is incorporated herein by reference in its entirety and with respect to fiber knobs.

In various embodiments, a vector of the present disclosure is an HDAd5/35 vector that includes Ad5 capsid proteins except that the fibers are chimeric in that they include an Ad5 fiber tail, an Ad35 fiber shaft, and an Ad35 fiber knob, optionally wherein the Ad35 fiber knob is mutated for increased affinity to CD46 (e.g., Ad5/35++). In particular embodiments, an Ad5/35++ vector is a chimeric Ad5/35 vector with a mutant Ad35++ fiber knob (see, e.g., Wang et al. 2008. J. Virol. 82 (21): 10567-79, which is incorporated herein by reference in its entirety and particularly with respect to fiber knob mutations). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593 (24): 3623-3648, 2019). In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

In various embodiments, a nucleic acid payload of the present disclosure (e.g., a nucleic acid payload encoding a gene editing systems as disclosed herein, can be too large for inclusion in certain vector systems, but the large capacity of certain adenoviral vectors and genomes can permit inclusion of such nucleic acid payloads in adenoviral vectors and genomes of the present disclosure. An additional advantage of adenoviral vectors and genomes in various embodiments is that adenoviral genomes do not naturally integrate into host cell genomes, which facilitates transient expression of gene editing systems and components (e.g., when present in a non-integrating fragment), which can be desirable, e.g., to avoid immunogenicity and/or genotoxicity (e.g., in connection with expression of a gene editing system).

### V. Applications

In vivo, in vitro, and or ex vivo modification of endogenous EpoR-encoding nucleic acids to encode signaling-enhanced EpoR can result in enrichment of modified cells (i.e., cells including the modified nucleic acids) by conferring a competitive advantage. The present disclosure includes the recognition that cells conferred a competitive advantage by expression of signaling-enhanced EpoR can survive and/or proliferate at a greater rate and/or frequency than reference cells that do not express signaling-enhanced EpoR. For example, in various embodiments HSCs and/or erythroid progenitors that express signaling-enhanced EpoR demonstrate a competitive advantage over reference cells, where the reference cells are HSCs and/or erythroid progenitors that do not express signaling-enhanced EpoR, such that the prevalence of modified cells expressing signaling-enhanced EpoR increases over time (e.g., as measured by the ratio of modified cells to reference cells, e.g., the ratio of modified HSCs and/or erythroid progenitors to reference and/or non-modified HSCs and/or erythroid progenitors). For at least this reason, modification of endogenous EpoR-encoding nucleic acids as disclosed herein is useful in increasing the in vivo, in vitro, and or ex vivo prevalence of modified cells (e.g., therapeutic cells) as compared to reference and/or non-modified cells, which enrichment can, in various embodiments, improve therapeutic efficacy.

In various embodiments, methods of the present disclosure that include modification of endogenous EpoR-encoding nucleic acids of target cells can include delivery of a nucleic acid payload disclosed herein to a subject, system, or cell. In various embodiments, methods of the present disclosure include delivery of a transgene encoding signaling-enhanced EpoR to a subject, system, or cell. In various embodiments, methods of the present disclosure that include modification of endogenous EpoR-encoding nucleic acids of target cells can include delivery of a nucleic acid payload encoding an editing system disclosed herein to a subject, system, or cell. In various embodiments, methods of the present disclosure that include modification of endogenous EpoR-encoding nucleic acids of target cells can include delivery of an editing system disclosed herein to a subject, system, or cell.

In various embodiments, a signaling-enhanced EpoR transgene is present in a nucleic acid payload that includes a therapeutic payload. In various embodiments, the signaling-enhanced EpoR transgene is in an integrating fragment or where the signaling enhanced EpoR transgene is in a non-integrating fragment. In various embodiments, an EpoR editing payload is present in a nucleic acid payload that includes a therapeutic payload. In various embodiments, the EpoR editing payload is present in a non-integrating fragment. In various embodiments, the therapeutic payload includes one or more components of an editing system that causes, elicits, or contributes to a desired pharmacological and/or physiological effect by editing a target nucleic acid sequence (a “therapeutic editing payload”). In various embodiments, the therapeutic payload includes one or more transgenes encoding a therapeutic polypeptide that is not a signaling-enhanced EpoR polypeptide and does not cause or contribute to editing of an endogenous EpoR nucleic acid.

At least in part because editing systems disclosed herein can be engineered to cause a wide variety of nucleic acid changes, editing systems disclosed herein are capable of treating a wide variety of genetic diseases, disorders, and conditions. To provide just one example, editing systems of the present disclosure can be used to treat a disease, disorder, or condition caused by a point mutation in the genomic DNA of a subject. Examples of diseases, disorders, and conditions that can result from point mutations include, without limitation Cystic Fibrosis, Sickle Cell Anemia, phenylketonuria, and Tay-Sachs. Those of skill in the art will appreciate, however, that therapeutic editing payloads can have many other types of targets.

As further examples of therapeutic editing targets, various therapeutic editing systems can target one or more nucleic acid sequences to cause increased expression of a globin polypeptide. In some embodiments a therapeutic gene editing payload encodes one or more components of a therapeutic gene editing system engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with a-globin polypeptide subunits. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34 (12): 927-940, 2018, which is incorporated herein by reference in its entirety and with respect to mutations that increase expression of γ-globin). Certain such mutations are found in the HBG1 promoter or HBG2 promoter.

In various embodiments, a therapeutic gene editing system that is designed to increase expression of γ-globin targets an HBG1/2 promoter and is designed to increase expression of γ-globin coding by modification and/or inactivation of a BCL11A repressor protein binding site. In various embodiments, a therapeutic gene editing system that is designed to increase expression of γ-globin targets the erythroid bcl11a enhancer and is designed to increase expression of γ-globin by modification and/or inactivation of the erythroid bcl11a enhancer to reduce BCL11A repressor protein expression in erythroid cells. In various embodiments, a therapeutic gene editing system that is designed to increase expression of γ-globin is targeted to cause a loss of function mutation in the gene encoding BCL11A.

In various embodiments, an EpoR editing payload is present in a nucleic acid that includes a therapeutic editing payload, where the EpoR editing system and therapeutic editing system are “multiplexed” in that the EpoR editing system and therapeutic editing system include and/or utilize the same editing enzyme encoded by the same nucleic acid fragment. Thus, in various embodiments, a nucleic acid encoding editing systems for both EpoR editing and therapeutic editing can encode a single editing enzyme that participates in and/or causes both the EpoR editing and the therapeutic editing. To provide one example, in various multiplexed embodiments, (i) an EpoR editing payload encodes an EpoR editing system that includes a base editing enzyme and a base editing gRNA, (ii) a therapeutic editing payload encodes a therapeutic base editing gRNA, and (iii) the EpoR editing and the therapeutic editing utilize (and/or the EpoR editing system and therapeutic editing system include) the same base editing enzyme. To provide another example, in various multiplexed embodiments, (i) an EpoR editing payload encodes an EpoR editing system that includes a prime editing enzyme and a pegRNA, (ii) a therapeutic editing payload encodes a therapeutic pegRNA, and (iii) the EpoR editing and the therapeutic editing utilize (and/or the EpoR editing system and therapeutic editing system include) the same prime editing enzyme. In various embodiments, one or more components of a multiplexed system can be operably linked to distinct regulatory elements (e.g., distinct promoters), operably linked to a single regulatory element (e.g., a single promoter), or each operably linked to separate copies of the same regulatory element (e.g., separate copies of the same promoter).

The present disclosure includes embodiments in which an EpoR editing payload is present in a nucleic acid that includes a therapeutic editing payload, where the EpoR editing system and therapeutic editing system are not multiplexed, in that the EpoR editing system and therapeutic editing system do not include and/or utilize any of the same editing system components (i.e., have no shared components, e.g., no shared editing enzyme). Thus, for example, a nucleic acid of the present disclosure can include an EpoR editing payload that encodes a base editing system and a therapeutic editing payload that encodes a prime editing system. To provide another example, an editing payload of the present disclosure can include an EpoR editing payload that encodes a prime editing system and a therapeutic editing payload that encodes a base editing system.

In various embodiments, an EpoR editing payload is present in a nucleic acid that also includes a therapeutic payload that does not encode an editing system. Exemplary expression products include proteins, including without limitation replacement therapy proteins for treatment of diseases or conditions characterized by low expression or activity of a biologically active protein as compared to a reference level. Exemplary expression products include antibodies, CARs, and TCRs. Exemplary expression products include small RNAs.

For the avoidance of doubt, in various embodiments a nucleic acid payload of the present disclosure does not include or encode any editing system, e.g., where a nucleic acid payload of the present disclosure includes a transgene that encodes signaling-enhanced EpoR and further includes a therapeutic payload that encodes a therapeutic agent that is not an editing system or component thereof.

In various embodiments, methods and compositions of the present disclosure (e.g., vectors and/or nucleic acid payloads) are delivered to target cells, where the target cells hare HSCs. For example, certain vectors target HSCs by binding CD46. In various embodiments, HSCs or subsets thereof can be identified by the presence of CD46. In various embodiments, HSCs or subsets thereof can be identified by any of the following marker profiles: CD34⁺; Lin⁻/CD34⁺/CD38⁻/CD45RA⁻/CD90⁺/CD49f⁺ (HSC1); CD34+/CD38⁻/CD45RA⁻/CD90⁻/CD49f⁺/(HSC2). In various embodiments, human HSCs can be identified by any of the following profiles: CD34⁺/CD38⁻/CD45RA⁻/CD90⁺ or CD34⁺/CD45RA⁻/CD90⁺ and mouse LT⁻ HSC can be identified by Lin⁻/Sca1⁺/ckit⁺/CD150⁺/CD48⁻/Flt3⁻/CD34− (where Lin⁻ represents the absence of expression of any marker of mature cells including CD3, CD4, CD8, CD11b, CD11c, NK1.1, Gr1, and TER119). In particular embodiments, HSCs are identified by a CD164⁺ profile. In particular embodiments, HSCs are identified by a CD34⁺/CD164⁺ profile. For additional information regarding HSC marker profiles, see WO 2017/218948, which is incorporated herein by reference.

Methods and compositions provided herein are disclosed at least in part for use in in vivo gene therapy. However, for the avoidance of doubt, the present disclosure expressly includes the use of compositions and methods provided herein for ex vivo engineering of cells and/or tissues, as well as in vitro uses including the engineering of cells and/or tissues for research purposes. Gene therapy includes use of a nucleic acid payload, vector, genome, or system of the present disclosure in a method of introducing exogenous DNA into a host cell (such as a target cell) and/or a nucleic acid (such as a target nucleic acid, such as a target genome, e.g., the genome of a target cell), which introducing of exogenous DNA can be referred to as genetic modification of the host cell or nucleic acid. Gene therapy can therefore be referred to herein, e.g., as a method of genetically modifying a host cell or nucleic acid. The present disclosure includes description and exemplification of compositions and methods relating to in vivo, in vitro, and ex vivo therapy. Those of skill in the art will appreciate from the present disclosure that that cells modified in vivo to encode and/or express signaling-enhanced EpoR will have a competitive advantage in the organism in which they were modified, and that cells modified in vitro, or ex vivo to encode and/or express signaling-enhanced EpoR can be subsequently administered to an organism in which they will have a competitive advantage over reference cells of the organism.

### V(A). In Vivo Gene Therapy

The present disclosure includes methods and compositions for in vivo gene therapy, which includes the direct delivery of a vector disclosed herein (e.g., without limitation, a viral vector, e.g., an adenoviral vector) to a subject. In vivo gene therapy is an attractive approach because it may not require any genotoxic conditioning (or could require less genotoxic conditioning) or ex vivo cell processing, and thus presents fewer barriers to adoption (e.g., at institutions worldwide, including those in developing countries). For example, various methods and compositions for in vivo gene therapy provided herein can include delivery of a vector (e.g., a viral vector, e.g., an adenoviral vector) by injection to a subject, which administration is similar to that already practice worldwide for the delivery of vaccines. In various embodiments, methods of in vivo gene therapy of the present disclosure can include one or more steps of (i) target cell mobilization, (ii) immunosuppression, (iii) administration of a vector, genome, system or formulation provided herein, and/or (iv) in certain embodiments in which a nucleic acid payload encodes a selection marker, selection of transduced cells and/or cells that have integrated an integrating fragment that encodes the selection marker.

In various embodiments, methods and compositions disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts of one or more vectors, genomes, or systems of the present disclosure.

Vectors disclosed herein can be administered in coordination with mobilization factors. In certain embodiments, a vector described herein can be administered in concert with HSC mobilization. In particular embodiments, administration of vector occurs concurrently with administration of one or more mobilization factors. In particular embodiments, administration of vector follows administration of one or more mobilization factors. In particular embodiments, administration of vector follows administration of a first one or more mobilization factors and occurs concurrently with administration of a second one or more mobilization factors. Agents for HSC mobilization include, for example, granulocyte-colony stimulating factor (G-CSF), granulocyte macrophage colony stimulating factor (GM-CSF), AMD3100, SCF, S-CSF, a CXCR4 antagonist, a CXCR2 agonist, and Gro-Beta (GRO-β). In various embodiments, a CXCR4 antagonist is AMD3100 and/or a CXCR2 agonist is GRO-β.

G-CSF is a cytokine whose functions in HSC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and compositions as disclosed herein, for example, Filgrastim (Neupogen®, Amgen Inc., Thousand Oaks, CA) and PEGylated Filgrastim (Pegfilgrastim, NEULASTA®, Amgen Inc., Thousand Oaks, CA).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and compositions as disclosed herein, for example, Sargramostim (Leukine, Bayer Healthcare Pharmaceuticals, Seattle, WA) and molgramostim (Schering-Plough, Kenilworth, NJ).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSC mobilization. AMD3100 is approved to be used in combination with G-CSF for HSC mobilization in patients with myeloma and lymphoma.

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and compositions as disclosed herein, for example, recombinant human SCF (Ancestim, STEMGEN®, Amgen Inc., Thousand Oaks, CA).

Chemotherapy used in intensive myelosuppressive treatments also mobilizes HSCs to the peripheral blood as a result of compensatory neutrophil production following chemotherapy-induced aplasia. In particular embodiments, chemotherapeutic agents that can be used for mobilization of HSCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (S1P) agonists (e.g., SEW2871, Juarez et al. Blood 119:707-716, 2012); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren et al. Blood 111:3893-3895, 2008); BIO5192, a small molecule inhibitor of VLA-4 (Ramirez et al. Blood 114:1340-1343, 2009)); parathyroid hormone (Brunner et al. Exp Hematol. 36:1157-1166, 2008); proteasome inhibitors (e.g., Bortezomib, Ghobadi et al. ASH Annual Meeting Abstracts. p. 583, 2012); Groβ, a member of CXC chemokine family which stimulates chemotaxis and activation of neutrophils by binding to the CXCR2 receptor (e.g., SB-251353, King et al. Blood 97:1534-1542, 2001); stabilization of hypoxia inducible factor (HIF) (e.g., FG-4497, Forristal et al. ASH Annual Meeting Abstracts. p. 216, 2012); Firategrast, an α4β1 and α4β7 integrin inhibitor (α4β1/7) (Kim et al. Blood 128:2457-2461, 2016); Vedolizumab, a humanized monoclonal antibody against the α4β7 integrin (Rosario et al. (lin Drug Investig 36:913-923, 2016); and BOP (N-(benzenesulfonyl)-L-prolyl-L-O-(1-pyrrolidinylcarbonyl) tyrosine) which targets integrins α9β1/α4β1 (Cao et al. Nat Commun 7:11007, 2016). Additional agents that can be used for HSC mobilization are described in, for example, Richter R et al. Transfus Med Hemother 44:151-164, 2017, Bendall & Bradstock, Cytokine & Growth Factor Reviews 25:355-367, 2014, WO 2003043651, WO 2005017160, WO 2011069336, U.S. Pat. Nos. 5,637,323, 7,288,521, 9,782,429, US 2002/0142462, and US 2010/02268.

In particular embodiments, a therapeutically effective amount of G-CSF includes 0.1 ug/kg to 100 μg/kg. In particular embodiments, a therapeutically effective amount of G-CSF includes 0.5 ug/kg to 50 μg/kg. In particular embodiments, a therapeutically effective amount of G-CSF includes 0.5 ug/kg, 1 μg/kg, 2 μg/kg, 3 μg/kg, 4 μg/kg, 5 μg/kg, 6 μg/kg, 7 g/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before vector delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to vector administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 ug/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before vector delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to vector administration. A dosing regimen for Sargramostim can include 200 ug/m2, 210 ug/m2, 220 ug/m2, 230 ug/m2, 240 g/m2, 250 ug/m2, 260 ug/m2, 270 ug/m2, 280 ug/m2, 290 ug/m2, 300 ug/m2, or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 ug/m2/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to vector administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 ug/kg 6 to 11 hours prior to vector delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to vector delivery.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 ug/kg/day, 1 μg/kg/day, 2 μg/kg/day, 3 μg/kg/day, 4 μg/kg/day, 5 g/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to vector administration.

In particular embodiments, growth factors GM-CSF and G-CSF can be administered to mobilize HSC in the bone marrow niches to the peripheral circulating blood to increase the fraction of HSCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US20140193376 describes mobilization protocols utilizing a CXCR4 antagonist with a SIP receptor 1 (SIPR1) modulator agent. US20110044997 describes mobilization protocols utilizing a CXCR4 antagonist with a vascular endothelial growth factor receptor (VEGFR) agonist.

Adenoviral vectors (e.g. Adenoviral vectors) are exemplary of vectors that can be administered in concert with HSC mobilization. In particular embodiments, administration of an adenoviral vector occurs concurrently with administration of one or more mobilization factors. In particular embodiments, administration of an Adenoviral vector follows administration of one or more mobilization factors. In particular embodiments, administration of an Adenoviral vector follows administration of a first one or more mobilization factors and occurs concurrently with administration of a second one or more mobilization factors.

In particular embodiments, an HSC enriching agent, such as a CD19 immunotoxin or 5-FU can be administered to enrich for HSCs. CD19 immunotoxin can be used to deplete all CD19 lineage cells, which accounts for 30% of bone marrow cells. Depletion encourages exit from the bone marrow. By forcing HSCs to proliferate (whether via, e.g., CD19 immunotoxin of 5-FU), this stimulates their differentiation and exit from the bone marrow and increases transgene marking in peripheral blood cells.

Therapeutically effective amounts of HSC mobilization factors and/or HSC enriching agents can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

In particular embodiments, methods of the present disclosure can include selection for cells modified to express a selection marker (e.g., a mutant form of MGMT that is resistant to inactivation by 6-BG, but retains the ability to repair DNA damage). For example, particular embodiments include regimens that combine mobilization (e.g., a mobilization protocol described herein) with administration of a vector described herein and administration

BCNU or benzylguanine and temozolomide in the case of an vector including a MGMT^{P140K} selection marker. In particular embodiments, the in vivo selection marker can include MGMT^{P140K} as described in Olszko et al., Gene Therapy 22:591-595, 2015. Thus, selection for cells that express MGMT^{P140K} can select for transduced cells and/or contribute to therapeutic efficacy.

A selection agent (e.g., an agent including an MGMT inhibitor, an alkylating agent, or a combination thereof) of the present disclosure can be formulated such that it is pharmaceutically acceptable for administration to cells or animals, e.g., to humans. A selection agent may be administered in vitro, ex vivo, or in vivo. The selection agents described herein can be formulated for administration to a subject. Formulations can include one or more pharmaceutically acceptable carriers.

Therapeutically effective amounts of a selection agent can include a dose of an MGMT inhibitor such as O⁶BG or an analog or derivative thereof that ranges from, for example, 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of MGMT inhibitor includes 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). Therapeutically effective amounts of a selection agent can include a dose of an alkylating agent such as BCNU or an analog or derivative thereof that ranges from, for example, 0.001 to 100 mg/kg (e.g., 1-5, 1-10, 1-20, 1-50, 5-10, 5-20, or 5-50 mg/kg). Therapeutically effective amounts of a selection agent can include a dose of an alkylating agent such as temozolamide or an analog or derivative thereof that ranges from, for example, 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of an alkylating agent includes 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of a selection agent can be administered subcutaneously or intravenously. In particular embodiments, a therapeutically effective amount of selection agent can be administered before, at the same time as, or after administration of one or more immunosuppression agents or immunosuppression regimens, one or more mobilization factors, one or more vectors, and/or one or more nucleic acids of the present disclosure.

Vectors can be administered concurrently with or following administration of one or more immunosuppression agents or immunosuppression regimens.

### V(B). In Vitro and Ex Vivo Gene Therapy

In vitro gene therapy includes use of a vector, genome, or system of the present disclosure in a method of introducing exogenous DNA into a host cell (such as a target cell), system (e.g., a plurality of cells including one or more target and/or host cells), and/or a nucleic acid (such as a target nucleic acid, such as a target genome), where the host cell, system, or nucleic acid is not present in a multicellular organism (e.g., in a laboratory). In some embodiments, a target cell, system, or nucleic acid is derived (e.g., as a biological sample or portion thereof) from a multicellular organism, such as a mammal (e.g., a mouse, rat, human, or non-human primate). In various embodiments, a system can include a plurality of cell types, including for example a plurality of hematopoietic cell types. In vitro engineering of a cell derived from a multicellular organism can be referred to as ex vivo engineering, and can be used in ex vivo therapy. In various embodiments, methods and compositions of the present disclosure are utilized, e.g., as disclosed herein, to modify a target cell or nucleic acid derived from a first multicellular organism and the engineered target cell or nucleic acid is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy.

Ex vivo cell therapies can include isolation of hematopoietic cells (e.g., stem, progenitor or differentiated cells) from a donor (e.g., a mammalian donor, e.g., a human donor) such as a patient or a normal and/or healthy donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue or to deliver a therapeutic agent to a disease site. In various ex vivo therapies there is no direct exposure of the subject to the gene transfer vector, and the target cells of transduction can be selected, expanded and/or differentiated, before or after any genetic engineering, to improve efficacy and safety.

Ex vivo therapies include haematopoietic cell transplantation. Autologous hematopoietic cell gene therapy represents a therapeutic option for several monogenic diseases of the blood and the immune system as well as for storage disorders, and it may become a first-line treatment option for selected disease conditions.

Applications of ex-vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo gene transfer to correct the deficiency. An example is provided by SCIDs, in which a deficiency in any one of several genes blocks the development of mature lymphoid cells. Transplantation of non-manipulated normal donor hematopoietic cells, which can in various embodiments allow generation of donor-derived functional haematopoietic cells of various lineages in the host, represents a therapeutic option for SCIDs, as well as many other diseases that affect the blood and immune system. Autologous hematopoietic cell gene therapy, which can include engineering of a target hematopoietic cell population and, similarly to allogenic hematopoietic cell transplantation, can provide a steady supply of functional hematopoietic cells (e.g., progeny of engineered hematopoietic stem and/or progenitor cells), may have several advantages, including reduced risk of graft versus host disease (GvHD), reduced risk of graft rejection, and reduced need for post-transplant immunosuppression.

Applications of ex-vivo therapy include augmenting therapeutic gene dosage. In some applications, hematopoietic cell gene therapy may augment the therapeutic efficacy of allogenic hematopoietic cell transplantation. Therapeutic gene dosage can be engineered to supra-normal levels in transplanted cells.

Applications of ex-vivo therapy include introducing novel function and targeting gene therapy. Ex vivo gene therapy can confer a novel function to hematopoietic cells (e.g., one or more particular types of hematopoietic cells) or their progeny, such as establishing drug resistance to allow administration of a high-dose antitumor chemotherapy regime or establishing resistance to a pre-established infection with a virus, such as HIV, or other pathogen by expressing RNA-based agents (for example, ribozymes, RNA decoys, antisense RNA, RNA aptamers and small interfering RNA) and protein-based agents (for example, dominant-negative mutant viral proteins, fusion inhibitors and engineered nucleases that target the pathogen's genome).

### V(C). Conditions Treatable by Gene Therapy

At least in part because vectors of the present disclosure (e.g. adenoviral vectors) can be used in vivo, in vitro, or ex vivo for modification of host and/or target cells, and further because a vector can include payloads encoding a wide variety of expression products, it will be clear from the present specification that various technologies provided herein have broad applicability and can be used to treat a wide variety of conditions. Examples of conditions treatable by administration of an adenoviral vector, genome, or system of the present disclosure include, without limitation genetic conditions (e.g., hemoglobinopathies) and conditions treatable by expression of a therapeutic polypeptide (e.g., cancer).

In various embodiments, methods and compositions of the present disclosure can be used to treat a genetic condition (e.g., a condition arising from and/or caused by a mutation present in the genome of one or more cells of a subject). In various embodiments, methods and compositions of the present disclosure can be used to treat a genetic condition arising from and/or caused by a single point mutation present in the genome of one or more cells of a subject (e.g., a heterozygous or homozygous single point mutation). In various embodiments, methods and compositions of the present disclosure can be used to treat a protein deficiency. In various embodiments, methods and compositions of the present disclosure can be used to treat an enzyme deficiency. In various embodiments, methods and compositions of the present disclosure can be used to treat a blood condition (e.g., a condition characterized by a blood cell abnormality). Examples of genetic (e.g., point mutation) conditions, protein deficiencies, enzyme deficiencies, and/or blood conditions that can be treated by methods and compositions of the present disclosure include adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenia purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III(Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, and/or β-thalassemia, β-thalassemia major.

In various embodiments, methods and compositions of the present disclosure can be used to treat an inborn error of metabolism. In various embodiments, methods and compositions of the present disclosure can be used to treat a hyperproliferative condition

In various embodiments, methods and compositions of the present disclosure can be used to treat a cancer (e.g., a cancer characterized by abnormal blood cells). Examples of cancers that can be treated by methods and compositions of the present disclosure include acute lymphoblastic leukemia (ALL), acute myelogenous leukemia (AML), agnogenic myeloid metaplasia, astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, chronic lymphocytic leukemia (CLL), chronic myelogenous leukemia (CML), clear cell sarcoma of soft tissue, diffuse large B-cell lymphoma, ependymoma, epithelioid sarcoma, Ewing sarcoma, extragonadal germ cell tumor, extrarenal rhabdoid tumor, follicular lymphoma, gastrointestinal stromal tumor, glioblastoma, HBV-induced hepatocellular carcinoma, head and neck cancer, Hodgkin's lymphoma, juvenile myelomonocytic leukemia, kidney cancer, lung cancer, lymphoma, malignant rhabdoid tumor, medulloblastoma, melanoma, meningioma, mesothelioma, multiple myeloma, myeloma, neuroglial tumor, non-Hodgkin's lymphoma, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullary carcinoma, rhabdomyosarcoma, sarcoma, schwannoma, skin squamous cell carcinoma, and/or stem cell cancer.

In various embodiments, methods and compositions of the present disclosure can be used to treat a hemoglobinopathy, red blood cell disorder, platelet disorder, and/or bone marrow disorder (e.g., a bone marrow failure condition).

In various embodiments, methods and compositions of the present disclosure can be used to treat an immune condition (e.g., an autoimmune condition). Examples of immune conditions (e.g., autoimmune conditions) that can be treated by methods and compositions of the present disclosure include acquired immunodeficiency syndrome (AIDS), acquired thrombotic thrombocytopenia purpura (aTTP), an autoimmune hematology, graft versus host disease (GVHD), Grave's Disease, inflammatory bowel disease, Multiple Sclerosis (MS), rheumatoid arthritis, severe aplastic anemia, and systemic lupus erythematosus (SLE).

In various embodiments, methods and compositions of the present disclosure can be used to treat an immunodeficiency (e.g., a primary immune deficiency, secondary immune deficiency, acquired immune deficiency, and/or an immune deficiency caused by trauma), an inflammatory condition, an IgG subclass deficiency, a complement disorders, or a specific antibody deficiency). In various embodiments, methods and compositions of the present disclosure can be used to eliminate or inhibit one or more subsets of lymphocytes (e.g., induce apoptosis in lymphocytes, inhibit lymphocyte activation, inhibit T cell activation, and/or inhibit Th-2 activity, and/or Th-1 activity), eliminate or inhibit autoreactive T cells, improve kinetics and/or clonal diversity of lymphocyte reconstitution, restore normal T lymphocyte development, restore thymic output, induce selective tolerance to an inciting agent, provide function to immune and other blood cells or treat an immune-mediated condition, In various embodiments, methods and compositions of the present disclosure can be used to normalize primary and secondary antibody responses to immunization.

In various embodiments, methods and compositions of the present disclosure can be used to treat and/or prevent an infection. In various embodiments, a composition of the present disclosure is a vaccine in that it encodes, and/or expresses in one or more cells of a subject, an antigen characteristic of an infectious agent (e.g., a viral or bacterial pathogen). In various embodiments, a method of the present disclosure is a method of vaccination in that it delivers to one or more cells of a subject an antigen characteristic of an infectious agent (e.g., a viral or bacterial pathogen) and/or induces an immune responses against the antigen and/or infectious agent. In various embodiments, a method or composition of the present disclosure delivers (e.g., causes transient expression of) an antigen in a subject. In various embodiments, a method or composition of the present disclosure is used to treat a subject that has the infection. In various embodiments, a method or composition of the present disclosure is used to treat a subject that is at risk of infection. In particular embodiments, the infectious disease is human immunodeficiency virus (HIV). A payload expression product can be, for example, an agent that renders a subject resistant to HIV infection, or which enables immune cells to effectively neutralize HIV. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

In various embodiments, a method or composition of the present disclosure delivers to one or more cells of a subject in need thereof a coding sequence that encodes and/or expresses a replacement polypeptide (i.e., a wild type, reference, and/or functional polypeptide that corresponds to a disease variant encoded by the genome of the subject). In various embodiments, a method or composition of the present disclosure delivers to one or more cells of a subject in need thereof an editing system that modifies a nucleic acid of the subject (e.g., a genome of the subject) to express and/or increase expression of a wild type, reference, and/or functional polypeptide, e.g., by correction of a disease mutation present in the nucleic acid of the subject.

Particular examples of conditions that can be treated by methods and compositions of the present disclosure include conditions in which mutation of a globin gene results in expression of an abnormal form of hemoglobin (e.g., as in sickle cell disease (SCD) or hemoglobin C, D, or E disease) or results in reduced production of the α or β polypeptides (and thus an imbalance of the globin chains in the cell). These latter conditions are termed α- or β-thalassemias, depending on which globin chain is impaired. 5% of the world population carries a significant hemoglobin variant with the sickle cell mutation in the b-globin (HBB) gene (a glutamate to valine conversion; historically E6V, contemporaneously E7V) being by far the most common (40% of carriers). The high prevalence and severity of hemoglobin disorders presents a substantial burden, impacting not only the lives of those affected but also health-care systems, since lifelong patient care is costly.

There are two forms of hemoglobin, fetal (HbF), which includes two alpha (α) and two gamma (γ) chains, and adult (HbA), which includes two a and two beta (β) chains. The natural switch from HbF to HbA occurs shortly after birth and is regulated by transcriptional repression of γ globin genes by factors including a master regulator, bcl11a. Critically, a variety of clinical observations demonstrate that the severity of β-hemoglobinopathies such as sickle cell disease and β-thalassemia are ameliorated by increased production of HbF.

In particular embodiments, a therapeutically effective treatment induces or increases expression of HbF, induces or increases production of hemoglobin and/or induces or increases production of β-globin. In particular embodiments, a therapeutically effective treatment improves blood cell function, and/or increases oxygenation of cells.

In various embodiments, the present disclosure includes treatment of a blood disorder using a vector of the present disclosure that includes a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein.

ET3 can have or include the following amino acid sequence: SEQ ID NO: 215. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to the SEQ ID NO: 215

(MQLELSTCVFLCLLPLGFSAIRRYYLGAVELSWDYRQSELLRELHVDTRFPATAPGALP
LGPSVLYKKTVFVEFTDQLFSVARPRPPWMGLLGPTIQAEVYDTVVVTLKNMASHPVSL
HAVGVSFWKSSEGAEYEDHTSQREKEDDKVLPGKSQTYVWQVLKENGPTASDPPCLTY
SYLSHVDLVKDLNSGLIGALLVCREGSLTRERTQNLHEFVLLFAVFDEGKSWHSARNDS
WTRAMDPAPARAQPAMHTVNGYVNRSLPGLIGCHKKSVYWHVIGMGTSPEVHSIFLEG
HTFLVRHHRQASLEISPLTFLTAQTFLMDLGQFLLFCHISSHHHGGMEAHVRVESCAEEP
QLRRKADEEEDYDDNLYDSDMDVVRLDGDDVSPFIQIRSVAKKHPKTWVHYIAAEEED
WDYAPLVLAPDDRSYKSQYLNNGPQRIGRKYKKVRFMAYTDETFKTREAIQHESGILGP
LLYGEVGDTLLIIFKNQASRPYNIYPHGITDVRPLYSRRLPKGVKHLKDFPILPGEIFKYK
WTVTVEDGPTKSDPRCLTRYYSSFVNMERDLASGLIGPLLICYKESVDQRGNQIMSDKR
NVILFSVFDENRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMSMENPGLWIL
GCHNSDFRNRGMTALLKVSSCDKNTGDYYEDSYEDISAYLLSKNNAIEPRSFAQNSRPP
SASAPKPPVLRRHQRDISLPTFQPEEDKMDYDDIFSTETKGEDFDIYGEDENQDPRSFQK
RTRHYFIAAVEQLWDYGMSESPRALRNRAQNGEVPRFKKVVFREFADGSFTQPSYRGE
LNKHLGLLGPYIRAEVEDNIMVTFKNQASRPYSFYSSLISYPDDQEQGAEPRHNFVQPNE
TRTYFWKVQHHMAPTEDEFDCKAWAYFSDVDLEKDVHSGLIGPLLICRANTLNAAHGR
QVTVQEFALFFTIFDETKSWYFTENVERNCRAPCHLQMEDPTLKENYRFHAINGYVMDT
LPGLVMAQNQRIRWYLLSMGSNENIHSIHFSGHVFSVRKKEEYKMAVYNLYPGVFETV
EMLPSKVGIWRIECLIGEHLQAGMSTTFLVYSKKCQTPLGMASGHIRDFQITASGQYGQ
WAPKLARLHYSGSINAWSTKEPFSWIKVDLLAPMIIHGIKTQGARQKFSSLYISQFIIMYS
LDGKKWQTYRGNSTGTLMVFFGNVDSSGIKHNIFNPPIIARYIRLHPTHYSIRSTLRMEL
MGCDLNSCSMPLGMESKAISDAQITASSYFTNMFATWSPSKARLHLQGRSNAWRPQVN
NPKEWLQVDFQKTMKVTGVTTQGVKSLLTSMYVKEFLISSSQDGHQWTLFFQNGKVK
VFQGNQDSFTPVVNSLDPPLLTRYLRIHPQSWVHQIALRMEVLGCEAQDLYV).

β-globin can have or include the following amino acid sequence: SEQ ID NO: 216. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 216

(MVHLTPEEKSAVTALWGKVNVDEVGGEALGRLLVVYPWTQRFFESFGD
LSTPDAVMGNPKVKAHGKKVLGAFSDGLAHLDNLKGTFATLSELHCDKL
HVDPENFRLLGNVLVCVLAHHFGKEFTPPVQAAYQKVVAGVANALAHKY
H).

γ-globin can have or include the following amino acid sequence: SEQ ID NO: 217. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 217

(MGHFTEEDKATITSLWGKVNVEDAGGETLGRLLVVYPWTQRFFDSFGN
LSSASAIMGNPKVKAHGKKVLTSLGDATKHLDDLKGTFAQLSELHCDKL
HVDPENFKLLGNVLVTVLAIHFGKEFTPEVQASWQKMVTAVASALSSRY
H).

### VI. Dosages, Formulations, and Administration

A vector can be formulated such that it is pharmaceutically acceptable for administration to cells or animals, e.g., to humans. A vector may be administered in vitro, ex vivo, or in vivo. Vectors described herein can be formulated for administration to a subject. Formulations can include a vector including a nucleic acid payload of the present disclosure and one or more pharmaceutically acceptable carriers. In various embodiments, the amount of vector and/or nucleic acid payload that is administered and/or introduced into a cell or organism is a therapeutically effective amount, is sufficient to provide one or more intended nucleic acid edits to at least one endogenous nucleic acid sequence, and/or is sufficient to provide an effective amount of an encoded expression product.

As disclosed herein, a vector can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Formulations disclosed herein can be formulated for administration by, for example, injection. For injection, a formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can include any other pharmaceutically acceptable carriers, e.g., other pharmaceutically acceptable carriers that do not produce significantly adverse, allergic, or other undesirable reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Therapeutically effective amounts of a viral vector (e.g., a viral vector that includes a nucleic acid payload of the present disclosure; e.g., an adenoviral vector) can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of a vector associated with a therapeutic gene includes 4×10⁸ IU. In particular embodiments, a therapeutically effective amount of a vector can be administered subcutaneously or intravenously. In particular embodiments, a therapeutically effective amount of a vector can be administered following administration with one or more mobilization factors.

In various embodiments, a unit dose, daily dose, or total dose of a vector, such as a viral vector or support vector, or the total combined dose of a viral vector and a support vector (if present and/or utilized), can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral vector or support vector, or the total combined dose of a viral vector and a support vector (if present and/or utilized), can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

In various embodiments, a viral vector is administered at a unit dose, daily dose, or total dose of at least 1E10, 5E10, 1E11, 5E11, 1E12, 5E12, 1E13, 5E13, 1E14, or 1E15 vp/kg and a support vector (if present and/or utilized) is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the viral vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the support vector (if present and/or utilized) is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various embodiments, a support vector is administered at a unit dose, daily dose, or total dose of at least 1E10, 5E10, 1E11, 5E11, 1E12, 5E12, 1E13, 5E13, 1E14, or 1E15 vp/kg and a supported viral vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the support vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the supported viral vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various compositions and methods including a supported vector and a support vector, the support vector is administered in an amount sufficient to provide a level of one or more encoded polypeptides (e.g., one or both of a transposase and/or a recombinase) that is sufficient to achieve one or more intended nucleic acid edits to at least one endogenous nucleic acid sequence, and/or to cause expression of an effective amount of an encoded expression product. In various embodiments, a supported vector and a support vector are administered in a pre-defined ratio. In various embodiments, the ratio is in the range of 2:1 to 1:2, e.g., 1:1. In various embodiments, a supported vector and a support vector are administered in a 1:1, 1:2, or 1:3 ratio of supported vector to support vector.

In in vivo gene therapy including more than one vector species, such as a first vector that is a supported vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments, a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments of the present disclosure, in vivo gene therapy includes administration of at least one vector to a subject in combination with at least one immune suppression regimen.

### VII. Kits

The present disclosure provides kits that include a nucleic acid payload of the present disclosure or a vector including the same (e.g., in a pharmaceutically acceptable formulation). A kit can optionally further include at least one additional composition for use in a method of gene therapy. In some embodiments, a kit of the present disclosure can include one or more hematopoietic cell mobilization agents. In some embodiments, a kit of the present disclosure can include one or more immunosuppression agents. In various embodiments, a kit can include instructions for administering a nucleic acid payload of the present disclosure or a vector including the same (e.g., in a pharmaceutically acceptable formulation) to a subject or system, e.g., to a mammalian subject.

### EXAMPLES

The present Examples illustrate the advantageous modification of endogenous nucleic acids that encode EpoR to produce modified nucleic acids that encode signaling-enhanced EpoR. As disclosed herein, such modified cells are characterized by a competitive advantage that causes an increase in the prevalence of modified cells (e.g., number of modified cells in a subject or system compared to a reference subject or system, ratio of modified cells to non-modified reference cells in a subject or system as compared to a reference subject or system, and/or number of modified cells in a subject or system compared to non-modified reference cells in the same subject or system). Because methods and compositions provided herein can increase the prevalence of modified cells, they are particularly useful e.g., in gene therapy as set forth herein.

### Example 1

The present Example includes use of base editing to modify an endogenous nucleic acid sequence that encodes EpoR to produce a modified nucleic acid that encodes signaling-enhanced EpoR. In particular, a G to A transition in the middle nucleotide of the W439 codon of the EpoR gene converts a TGG codon for tryptophan into a TAG stop codon, generating a truncation of the 70 C-terminal amino acids of EpoR. Cytosine base editors, which convert C⋅G base pairs to T⋅A base pairs, will be able to introduce this modification upon in vivo delivery and/or expression in HSCs or their progeny. In the event of a bystander mutation caused by the CBE base editor, the adjacent G would be converted to an A. This would result in a TAA stop codon. Monoallelic conversion is expected to be sufficient to confer a competitive advantage, as benign erythrocytosis and elevated hematocrit levels have been observed in subjects heterozygous for the truncating EpoR variant. Exemplary cytosine base editor reagents for producing the G to A transition in the middle nucleotide of the W439 codon can include:
CRISPR-Cas orthologue: SpCas9-EQR from Streptococcus pyogenes: 5′-NGAG-3′
CRISPR Target (5′ to 3′): GTCCATGGACGCAAGAGCTGGGAG (SEQ ID NO: 219) (Underline: PAM site; Bold: Editing window)
   Editing outcome: Trp439Ter (Ter=stop codon)

### Example 2

The present Example includes use of prime editing to modify an endogenous nucleic acid sequence that encodes EpoR to produce a modified nucleic acid that encodes signaling-enhanced EpoR. In particular, a 42-residue deletion from the C-terminus can be produced via prime editing to introduce a stop codon. The present Example illustrates, among other things, that more than one prime editor system can be used to generate a particular EpoR modification.

pegRNAs can be generated by introducing user-designed target-specific nucleic acids into a pegRNA acceptor plasmid. The present Example utilizes the publicly available acceptor plasmid pU6-pegRNA-GG-acceptor (Addgene plasmid #132777). The pegRNA acceptor plasmid encodes and can express a pegRNA following introduction into the plasmid of a spacer and a 3′ extension (including the PBS and RT template).

A secondary nicking sgRNA can also optionally be included to stimulate re-synthesis of the non-edited strand using the edited strand as a template, resulting in a fully edited duplex. sgRNAs can be generated by introducing user-designed target-specific nucleic acids into a sgRNA acceptor plasmid. The present Example utilizes the publicly available sgRNA acceptor plasmid, PE3. The sgRNA acceptor plasmid encodes and can express a sgRNA following introduction into the plasmid of a spacer sequence targeting the site to be nicked.

Exemplary prime editor reagents can be selected from either of the following examples.

In certain embodiments, the prime editor is designed using Cas9-NG to generate Asp467Ter mutation using TAG as stop codon according to Tables 24-27. The present Example includes pegRNAs characterized by a spacer selected from Table 24, an RT template selected from Table 25, and a PBS sequence selected from Table 26 in the following tables. Information presented in Table 24 additionally includes the strand orientation of the spacer sequence (whether a portion of a sequence that corresponds to the spacer sequence is present in a “sense” or “antisense” strand, e.g., relative to a sequence encoding all or a portion of an EpoR polypeptide), the distance between the spacer sequence and the nearest edited nucleotide of the target sequence, and whether the PAM site is disrupted. The present Example additionally includes secondary nicking sgRNAs characterized by a sequence selected from Table 27.

TABLE 24
Spacers

| | | Distance | | SEQ |
|---|---|---|---|---|
| | | to Edit | Seed/PAM | ID |
| Sequence | Orientation | Start | Disrupt | NO: |
| AGTCCCCTGAGCTGTAGTC | antisense | 0 | 1 | 219 |
| CTGGGAGTCCCCTGAGCTGT | antisense | 4 | 1 | 220 |
| TCTGACTCTGGCATCTCAAC | sense | 4 | 1 | 221 |
| TCCCTGGGAGTCCCCTGAGC | antisense | 7 | 0 | 222 |
| GGCTCCCTGGGAGTCCCCTG | antisense | 10 | 0 | 223 |
| TGGGCTCCCTGGGAGTCCCC | antisense | 12 | 0 | 224 |
| ACCTTGTGGTATCTGACTCT | sense | 15 | 0 | 225 |
| TACCTTGTGGTATCTGACTC | sense | 16 | 0 | 226 |
| GCCCCCTTGGGCTCCCTGGG | antisense | 19 | 0 | 227 |
| AAGCCCCCTTGGGCTCCCTG | antisense | 21 | 0 | 228 |
| TAAGCCCCCTTGGGCTCCCT | antisense | 22 | 0 | 229 |
| TACCTGTACCTTGTGGTATC | sense | 22 | 0 | 230 |
| ATAAGCCCCCTTGGGCTCCC | antisense | 23 | 0 | 231 |
| CTAAAGTACCTGTACCTTGT | sense | 28 | 0 | 232 |
| CCTAAAGTACCTGTACCTTG | sense | 29 | 0 | 233 |
| CCATCGGATAAGCCCCCTTG | antisense | 30 | 0 | 234 |
| CACCTAAAGTACCTGTACCT | sense | 31 | 0 | 235 |
| GCCATCGGATAAGCCCCCTT | antisense | 31 | 0 | 236 |
| GGCCATCGGATAAGCCCCCT | antisense | 32 | 0 | 237 |
| CCCACCCCACCTAAAGTACC | sense | 38 | 0 | 238 |
| GGAGTAGGGGCCATCGGATA | antisense | 40 | 0 | 239 |
| CCCTACCCCACCCCACCTAA | sense | 44 | 0 | 240 |

TABLE 25
RT Templates

| | | SEQ ID |
|---|---|---|
| Sequence | Length | NO: |
| CTCAACTTAG | 10 | 241 |
| TCTCAACTTAG | 11 | 242 |
| ATCTCAACTTAG | 12 | 243 |
| CATCTCAACTTAG | 13 | 244 |
| GCATCTCAACTTAG | 14 | 245 |
| GGCATCTCAACTTAG | 15 | 246 |
| TGGCATCTCAACTTAG | 16 | 247 |

TABLE 26
PBS Sequences

| | | SEQ ID |
|---|---|---|
| Sequence | Length | NO: |
| TACAGCTC | 8 | 248 |
| TACAGCTCA | 9 | 249 |
| TACAGCTCAG | 10 | 250 |
| TACAGCTCAGG | 11 | 251 |
| TACAGCTCAGGG | 12 | 252 |
| TACAGCTCAGGGG | 13 | 253 |
| TACAGCTCAGGGGA | 14 | 254 |
| TACAGCTCAGGGGAC | 15 | 255 |
| TACAGCTCAGGGGACT | 16 | 256 |
| TACAGCTCAGGGGACTC | 17 | 257 |

TABLE 27
Secondary Nicking sgRNA Sequences

| | | | Distance | |
|---|---|---|---|---|
| | | | to | |
| | Nick | | pegRNA | SEQ ID |
| Sequence | Position | Orientation | Nick | NO: |
| GCATCTCAACTTAGTAC | 118 | sense | 0 | 258 |
| TGACTCTGGCATCTCAACTT | 113 | sense | 5 | 259 |
| ATCTCAACTTAGTACAGCTC | 123 | sense | −5 | 260 |
| TCTCAACTTAGTACAGCTCA | 124 | sense | −6 | 261 |
| CCCTACCCCACCCCACCTAA | 71 | sense | 45 | 262 |
| ATGGACACTGTGCCCTGAGC | 47 | sense | 69 | 263 |
| TCCATGGACACTGTGCCCTG | 44 | sense | 72 | 264 |
| CGTCCATGGACACTGTGCCC | 42 | sense | 74 | 265 |
| TCTTGCGTCCATGGACACTG | 37 | sense | 79 | 266 |
| GCTCTTGCGTCCATGGACAC | 35 | sense | 81 | 267 |
| CTCCCAGCTCTTGCGTCCAT | 29 | sense | 87 | 268 |
| GCTCCCAGCTCTTGCGTCCA | 28 | sense | 88 | 269 |
| ACCCCAGCTCCCAGCTCTTG | 22 | sense | 94 | 270 |
| GGACCCCAGCTCCCAGCTCT | 20 | sense | 96 | 271 |
| GGAGCCCAAGGGGGCTTATC | 156 | sense | −40 | 272 |
| GCCCAAGGGGGCTTATCCGA | 159 | sense | −43 | 273 |
| CCCAAGGGGGCTTATCCGAT | 160 | sense | −44 | 274 |

In certain embodiments, the prime editor is designed using Cas9-SpRY to generate Asp467Ter mutation using TAG as stop codon according to Tables 28-31. The present Example includes pegRNAs characterized by a spacer selected from Table 28, an RT template selected from Table 29, and a PBS sequence selected from Table 30 in the following tables. Information presented in Table 28 additionally includes the strand orientation of the spacer sequence (whether a portion of a sequence that corresponds to the spacer sequence is present in a “sense” or “antisense” strand, e.g., relative to a sequence encoding all or a portion of an EpoR polypeptide), the distance between the spacer sequence and the nearest edited nucleotide of the target sequence, and whether the PAM site is disrupted. The present Example additionally includes secondary nicking sgRNAs characterized by a sequence selected from Table 31.

TABLE 28
Spacers

| | | Distance | | SEQ |
|---|---|---|---|---|
| | | to Edit | Seed/PAM | ID |
| Sequence | Orientation | Start | Disrupt | NO: |
| GAGTCCCCTGAGCTGTAGTC | antisense | 0 | 1 | 275 |
| ACTCTGGCATCTCAACTGAC | sense | 0 | 1 | 276 |
| GGAGTCCCCTGAGCTGTAGT | antisense | 1 | 1 | 277 |
| GACTCTGGCATCTCAACTGA | sense | 1 | 1 | 278 |
| GGGAGTCCCCTGAGCTGTAG | antisense | 2 | 1 | 279 |
| TGACTCTGGCATCTCAACTG | sense | 2 | 1 | 280 |
| CTGACTCTGGCATCTCAACT | sense | 3 | 0 | 281 |
| TGGGAGTCCCCTGAGCTGTA | antisense | 3 | 0 | 282 |
| CTGGGAGTCCCCTGAGCTGT | antisense | 4 | 0 | 283 |
| TCTGACTCTGGCATCTCAAC | sense | 4 | 0 | 284 |
| CCTGGGAGTCCCCTGAGCTG | antisense | 5 | 0 | 285 |
| ATCTGACTCTGGCATCTCAA | sense | 5 | 0 | 286 |
| CCCTGGGAGTCCCCTGAGCT | antisense | 6 | 0 | 287 |
| TATCTGACTCTGGCATCTCA | sense | 6 | 0 | 288 |
| TCCCTGGGAGTCCCCTGAGC | antisense | 7 | 0 | 289 |
| GTATCTGACTCTGGCATCTC | sense | 7 | 0 | 290 |
| GGTATCTGACTCTGGCATCT | sense | 8 | 0 | 291 |
| CTCCCTGGGAGTCCCCTGAG | antisense | 8 | 0 | 292 |
| GCTCCCTGGGAGTCCCCTGA | antisense | 9 | 0 | 293 |
| TGGTATCTGACTCTGGCATC | sense | 9 | 0 | 294 |
| GTGGTATCTGACTCTGGCAT | sense | 10 | 0 | 295 |
| GGCTCCCTGGGAGTCCCCTG | antisense | 10 | 0 | 296 |
| TGTGGTATCTGACTCTGGCA | sense | 11 | 0 | 297 |
| GGGCTCCCTGGGAGTCCCCT | antisense | 11 | 0 | 298 |
| TGGGCTCCCTGGGAGTCCCC | antisense | 12 | 0 | 299 |
| TTGTGGTATCTGACTCTGGC | sense | 12 | 0 | 300 |
| TTGGGCTCCCTGGGAGTCCC | antisense | 13 | 0 | 301 |
| CTTGTGGTATCTGACTCTGG | sense | 13 | 0 | 302 |
| CCTTGTGGTATCTGACTCTG | sense | 14 | 0 | 303 |
| CTTGGGCTCCCTGGGAGTCC | antisense | 14 | 0 | 304 |
| CCTTGGGCTCCCTGGGAGTC | antisense | 15 | 0 | 305 |
| ACCTTGTGGTATCTGACTCT | sense | 15 | 0 | 306 |
| CCCTTGGGCTCCCTGGGAGT | antisense | 16 | 0 | 307 |
| TACCTTGTGGTATCTGACTC | sense | 16 | 0 | 308 |
| CCCCTTGGGCTCCCTGGGAG | antisense | 17 | 0 | 309 |
| GTACCTTGTGGTATCTGACT | sense | 17 | 0 | 310 |
| TGTACCTTGTGGTATCTGAC | sense | 18 | 0 | 311 |
| CCCCCTTGGGCTCCCTGGGA | antisense | 18 | 0 | 312 |

TABLE 29
RT Templates

| | | SEQ ID |
|---|---|---|
| Sequence | Length | NO: |
| CTCAACTTAG | 10 | 313 |
| TCTCAACTTAG | 11 | 314 |
| ATCTCAACTTAG | 12 | 315 |
| CATCTCAACTTAG | 13 | 316 |
| GCATCTCAACTTAG | 14 | 317 |
| GGCATCTCAACTTAG | 15 | 318 |
| TGGCATCTCAACTTAG | 16 | 319 |

TABLE 30
PBS Sequences

| | | SEQ ID |
|---|---|---|
| Sequence | Length | NO: |
| TACAGCTC | 8 | 320 |
| TACAGCTCA | 9 | 321 |
| TACAGCTCAG | 10 | 322 |
| TACAGCTCAGG | 11 | 323 |
| TACAGCTCAGGG | 12 | 324 |
| TACAGCTCAGGGG | 13 | 325 |
| TACAGCTCAGGGGA | 14 | 326 |
| TACAGCTCAGGGGAC | 15 | 327 |
| TACAGCTCAGGGGACT | 16 | 328 |
| TACAGCTCAGGGGACTC | 17 | 329 |

TABLE 31
Secondary Nicking sgRNA Sequences

| | | | Distance | |
|---|---|---|---|---|
| | | | to | SEQ |
| | Nick | | pegRNA | ID |
| Sequence | Position | Orientation | Nick | NO: |
| CTGGCATCTCAACTTAGTAC | 118 | sense | 0 | 330 |
| TCTGGCATCTCAACTTAGTA | 117 | sense | 1 | 331 |
| TGGCATCTCAACTTAGTACA | 119 | sense | −1 | 332 |
| CTCTGGCATCTCAACTTAGT | 116 | sense | 2 | 333 |
| GGCATCTCAACTTAGTACAG | 120 | sense | −2 | 334 |
| ACTCTGGCATCTCAACTTAG | 115 | sense | 3 | 335 |
| GCATCTCAACTTAGTACAGC | 121 | sense | −3 | 336 |
| GACTCTGGCATCTCAACTTA | 114 | sense | 4 | 337 |
| CATCTCAACTTAGTACAGCT | 122 | sense | −4 | 338 |
| ATCTCAACTTAGTACAGCTC | 123 | sense | −5 | 339 |
| TGACTCTGGCATCTCAACTT | 113 | sense | 5 | 340 |
| TCTCAACTTAGTACAGCTCA | 124 | sense | −6 | 341 |
| CTGCCCCCTACCCCACCCCA | 66 | sense | 50 | 342 |
| GCTGCCCCCTACCCCACCCC | 65 | sense | 51 | 343 |
| TGCCCCCTACCCCACCCCAC | 67 | sense | 49 | 344 |
| AGCTGCCCCCTACCCCACCC | 64 | sense | 52 | 345 |
| GCCCCCTACCCCACCCCACC | 68 | sense | 48 | 346 |
| GAGCTGCCCCCTACCCCACC | 63 | sense | 53 | 347 |
| CCCCCTACCCCACCCCACCT | 69 | sense | 47 | 348 |
| CCCCTACCCCACCCCACCTA | 70 | sense | 46 | 349 |
| TGAGCTGCCCCCTACCCCAC | 62 | sense | 54 | 350 |
| CTGAGCTGCCCCCTACCCCA | 61 | sense | 55 | 351 |
| CCCTACCCCACCCCACCTAA | 71 | sense | 45 | 352 |
| CCTACCCCACCCCACCTAAA | 72 | sense | 44 | 353 |
| CCTGAGCTGCCCCCTACCCC | 60 | sense | 56 | 354 |
| CCCTGAGCTGCCCCCTACCC | 59 | sense | 57 | 355 |
| CTACCCCACCCCACCTAAAG | 73 | sense | 43 | 356 |
| TACCCCACCCCACCTAAAGT | 74 | sense | 42 | 357 |
| GCCCTGAGCTGCCCCCTACC | 58 | sense | 58 | 358 |
| TGCCCTGAGCTGCCCCCTAC | 57 | sense | 59 | 359 |
| ACCCCACCCCACCTAAAGTA | 75 | sense | 41 | 360 |
| CCCCACCCCACCTAAAGTAC | 76 | sense | 40 | 361 |
| GTGCCCTGAGCTGCCCCCTA | 56 | sense | 60 | 362 |
| TGTGCCCTGAGCTGCCCCCT | 55 | sense | 61 | 363 |
| CTGTGCCCTGAGCTGCCCCC | 54 | sense | 62 | 364 |
| ACTGTGCCCTGAGCTGCCCC | 53 | sense | 63 | 365 |
| CACTGTGCCCTGAGCTGCCC | 52 | sense | 64 | 366 |
| ACACTGTGCCCTGAGCTGCC | 51 | sense | 65 | 367 |
| GACACTGTGCCCTGAGCTGC | 50 | sense | 66 | 368 |
| GGACACTGTGCCCTGAGCTG | 49 | sense | 67 | 369 |
| TGGACACTGTGCCCTGAGCT | 48 | sense | 68 | 370 |
| ATGGACACTGTGCCCTGAGC | 47 | sense | 69 | 37 |
| CATGGACACTGTGCCCTGAG | 46 | sense | 70 | 372 |
| CCATGGACACTGTGCCCTGA | 45 | sense | 71 | 373 |
| TCCATGGACACTGTGCCCTG | 44 | sense | 72 | 374 |
| GTCCATGGACACTGTGCCCT | 43 | sense | 73 | 375 |
| CGTCCATGGACACTGTGCCC | 42 | sense | 74 | 376 |
| GCGTCCATGGACACTGTGCC | 41 | sense | 75 | 377 |
| TGCGTCCATGGACACTGTGC | 40 | sense | 76 | 378 |
| TTGCGTCCATGGACACTGTG | 39 | sense | 77 | 379 |
| CTTGCGTCCATGGACACTGT | 38 | sense | 78 | 380 |
| TCTTGCGTCCATGGACACTG | 37 | sense | 79 | 381 |
| CTCTTGCGTCCATGGACACT | 36 | sense | 80 | 382 |
| GCTCTTGCGTCCATGGACAC | 35 | sense | 81 | 383 |
| AGCTCTTGCGTCCATGGACA | 34 | sense | 82 | 384 |
| CAGCTCTTGCGTCCATGGAC | 33 | sense | 83 | 385 |
| CCAGCTCTTGCGTCCATGGA | 32 | sense | 84 | 386 |
| CCCAGCTCTTGCGTCCATGG | 31 | sense | 85 | 387 |
| TCCCAGCTCTTGCGTCCATG | 30 | sense | 86 | 388 |
| CTCCCAGCTCTTGCGTCCAT | 29 | sense | 87 | 389 |
| GCTCCCAGCTCTTGCGTCCA | 28 | sense | 88 | 390 |
| AGCTCCCAGCTCTTGCGTCC | 27 | sense | 89 | 391 |
| CAGCTCCCAGCTCTTGCGTC | 26 | sense | 90 | 392 |
| CCAGCTCCCAGCTCTTGCGT | 25 | sense | 91 | 393 |
| CCCAGCTCCCAGCTCTTGCG | 24 | sense | 92 | 394 |
| CCCCAGCTCCCAGCTCTTGC | 23 | sense | 93 | 395 |
| ACCCCAGCTCCCAGCTCTTG | 22 | sense | 94 | 396 |
| GACCCCAGCTCCCAGCTCTT | 21 | sense | 95 | 397 |
| GGACCCCAGCTCCCAGCTCT | 20 | sense | 96 | 398 |
| TGGACCCCAGCTCCCAGCTC | 19 | sense | 97 | 399 |
| CTGGACCCCAGCTCCCAGCT | 18 | sense | 98 | 400 |
| GGAGCCCAAGGGGGCTTATC | 156 | sense | −40 | 401 |
| GAGCCCAAGGGGGCTTATCC | 157 | sense | −41 | 402 |
| AGCCCAAGGGGGCTTATCCG | 158 | sense | −42 | 403 |
| GCCCAAGGGGGCTTATCCGA | 159 | sense | −43 | 404 |
| CCCAAGGGGGCTTATCCGAT | 160 | sense | −44 | 405 |
| CCAAGGGGGCTTATCCGATG | 161 | sense | −45 | 406 |
| CAAGGGGGCTTATCCGATGG | 162 | sense | −46 | 407 |
| AAGGGGGCTTATCCGATGGC | 163 | sense | −47 | 408 |
| AGGGGGCTTATCCGATGGCC | 164 | sense | −48 | 409 |
| GGGGGCTTATCCGATGGCCC | 165 | sense | −49 | 410 |
| GGGGCTTATCCGATGGCCCC | 166 | sense | −50 | 411 |
| GGGCTTATCCGATGGCCCCT | 167 | sense | −51 | 412 |
| GGCTTATCCGATGGCCCCTA | 168 | sense | −52 | 413 |
| GCTTATCCGATGGCCCCTAC | 169 | sense | −53 | 414 |
| CTTATCCGATGGCCCCTACT | 170 | sense | −54 | 415 |

### Example 3

The present Example includes use of base editing to modify an endogenous nucleic acid sequence that encodes EpoR to produce a modified nucleic acid that encodes signaling-enhanced EpoR. In particular, a 41-residue truncation that deletes the tyrosine at position 468 can be produced using the CGBE1 C-to-G editor. The present Example illustrates, among other things, that more than one base editor system can be used to generate a particular EpoR modification. Exemplary cytosine base editor reagents for producing a 41-residue truncation can include either of:

(i)
CRISPR-Cas orthologue: SpCas9: 5′-NGG-3′
CRISPR Target (5′ to 3′):
(SEQ ID NO: 652)
ATCTCAACTGACTACAGCTCAGG
Underline: PAM site
Bold: Editing window
Editing outcome: Tyr468Ter (Ter = stop codon)
(ii)
CRISPR-Cas orthologue: SpCas9-NG (modified PAM specificity)
CRISPR Target (5′ to 3′):
(SEQ ID NO: 653)
ATCTCAACTGACTACAGCTCAG
Underline: PAM site
Bold: Editing window
Editing outcome: Tyr468Ter (Ter = stop codon)

### Example 4

The present Example includes use of prime editing to modify an endogenous nucleic acid sequence that encodes EpoR to produce a modified nucleic acid that encodes signaling-enhanced EpoR. In particular, a high affinity binding site for SOCS-3, a negative regulator of EpoR, at the double phosphorylated motif pY454pY456 that can be advantageously modified based at least in part on the proximity of the two tyrosine residues which may function together in a phosphorylation-dependent manner (e.g., synergistically) in EpoR signaling. Prime editing to induce a substitution of these two residues with phenylalanine will reduce the SOCS-3 interaction. The present Example illustrates, among other things, that more than one prime editor system can be used to generate a particular EpoR modification. TTC was selected as the phenylalanine codon given it is the most common codon for phenylalanine in the human genome.

pegRNAs can be generated by introducing user-designed target-specific nucleic acids into a pegRNA acceptor plasmid. The present Example utilizes the publicly available acceptor plasmid pU6-pegRNA-GG-acceptor (Addgene plasmid #132777). The pegRNA acceptor plasmid encodes and can express a pegRNA following introduction into the plasmid of a spacer and a 3′ extension (including the PBS and RT template).

A secondary nicking sgRNA can also optionally be included to stimulate re-synthesis of the non-edited strand using the edited strand as a template, resulting in a fully edited duplex. sgRNAs can be generated by introducing user-designed target-specific nucleic acids into a sgRNA acceptor plasmid. The present Example utilizes the publicly available sgRNA acceptor plasmid, PE3. The sgRNA acceptor plasmid encodes and can express a sgRNA following introduction into the plasmid of a spacer sequence targeting the site to be nicked.

Exemplary prime editor reagents for producing a 41-residue truncation can be selected from either of the following examples:

In certain embodiments, the prime editor is designed using Cas9-NG to generate YLY(454-456)FLF mutation using TTC as a phenylalanine codon according to Tables 32-35. The present Example includes pegRNAs characterized by a spacer selected from Table 32, an RT template selected from Table 33, and a PBS sequence selected from Table 34 in the following tables. Information presented in Table 32 additionally includes the strand orientation of the spacer sequence (whether a portion of a sequence that corresponds to the spacer sequence is present in a “sense” or “antisense” strand, e.g., relative to a sequence encoding all or a portion of an EpoR polypeptide), the distance between the spacer sequence and the nearest edited nucleotide of the target sequence, and whether the PAM site is disrupted. The present Example additionally includes secondary nicking sgRNAs characterized by a sequence selected from Table 35.

TABLE 32
Spacers

| | | Distance | | |
|---|---|---|---|---|
| | | to Edit | Seed/PAM | SEQ ID |
| Sequence | Orientation | Start | Disrupt | NO: |
| AGTCAGATACCACAAGGTAC | antisense | 0 | 1 | 416 |
| CCCACCCCACCTAAAGTACC | sense | 0 | 1 | 417 |
| GCCAGAGTCAGATACCACAA | antisense | 5 | 0 | 418 |
| CCCTACCCCACCCCACCTAA | sense | 6 | 0 | 419 |
| TGCCAGAGTCAGATACCACA | antisense | 6 | 0 | 420 |
| TCAGTTGAGATGCCAGAGTC | antisense | 16 | 0 | 421 |
| GTAGTCAGTTGAGATGCCAG | antisense | 20 | 0 | 422 |
| CTGTAGTCAGTTGAGATGCC | antisense | 22 | 0 | 423 |
| TGAGCTGTAGTCAGTTGAGA | antisense | 26 | 0 | 424 |
| CCCTGAGCTGTAGTCAGTTG | antisense | 29 | 0 | 425 |
| ATGGACACTGTGCCCTGAGC | sense | 30 | 0 | 426 |
| TCCCCTGAGCTGTAGTCAGT | antisense | 31 | 0 | 427 |
| TCCATGGACACTGTGCCCTG | sense | 33 | 0 | 428 |
| GAGTCCCCTGAGCTGTAGTC | antisense | 34 | 0 | 429 |
| CGTCCATGGACACTGTGCCC | sense | 35 | 0 | 430 |
| CTGGGAGTCCCCTGAGCTGT | antisense | 38 | 0 | 431 |
| TCTTGCGTCCATGGACACTG | sense | 40 | 0 | 432 |
| TCCCTGGGAGTCCCCTGAGC | antisense | 41 | 0 | 433 |
| GCTCTTGCGTCCATGGACAC | sense | 42 | 0 | 434 |
| GGCTCCCTGGGAGTCCCCTG | antisense | 44 | 0 | 435 |

TABLE 33
RT Templates

| | | SEQ ID |
|---|---|---|
| Sequence | Length | NO: |
| AGTTCCTGTT | 10 | 436 |
| AAGTTCCTGTT | 11 | 437 |
| AAAGTTCCTGTT | 12 | 438 |
| TAAAGTTCCTGTT | 13 | 439 |
| CTAAAGTTCCTGTT | 14 | 440 |
| CCTAAAGTTCCTGTT | 15 | 441 |
| ACCTAAAGTTCCTGTT | 16 | 442 |

TABLE 34
PBS Sequences

| | | SEQ ID |
|---|---|---|
| Sequence | Length | NO: |
| CCTTGTGG | 8 | 443 |
| CCTTGTGGT | 9 | 444 |
| CCTTGTGGTA | 10 | 445 |
| CCTTGTGGTAT | 11 | 446 |
| CCTTGTGGTATC | 12 | 447 |
| CCTTGTGGTATCT | 13 | 448 |
| CCTTGTGGTATCTG | 14 | 449 |
| CCTTGTGGTATCTGA | 15 | 450 |
| CCTTGTGGTATCTGAC | 16 | 451 |
| CCTTGTGGTATCTGACT | 17 | 452 |

TABLE 35
Secondary Nicking sgRNA Sequences

| | | | Distance | |
|---|---|---|---|---|
| | Nick | | to | SEQ |
| | Pos- | Orien- | pegRNA | ID |
| Sequence | ition | tation | Nick | NO: |
| CACCTAAAGTTCCTGTTCCT | 110 | sense | 0 | 453 |
| CCTAAAGTTCCTGTTCCTTG | 112 | sense | -2 | 454 |
| CTAAAGTTCCTGTTCCTTGT | 113 | sense | -3 | 455 |
| CCCACCCCACCTAAAGTTCC | 103 | sense | 7 | 456 |
| CTCCCAGCTCTTGCGTCCAT | 55 | sense | 53 | 457 |
| GCTCTTGCGTCCATGGACAC | 61 | sense | 47 | 458 |
| GCTCCCAGCTCTTGCGTCCA | 54 | sense | 54 | 459 |
| TCTTGCGTCCATGGACACTG | 63 | sense | 45 | 460 |
| ACCCCAGCTCCCAGCTCTTG | 48 | sense | 60 | 461 |
| CGTCCATGGACACTGTGCCC | 68 | sense | 40 | 462 |
| GGACCCCAGCTCCCAGCTCT | 46 | sense | 62 | 463 |
| TATCCTGGACCCCAGCTCCC | 40 | sense | 68 | 464 |
| AGTACACTATCCTGGACCCC | 33 | sense | 75 | 465 |
| AGCTTTGAGTACACTATCCT | 26 | sense | 82 | 466 |
| CAGCTTTGAGTACACTATCC | 25 | sense | 83 | 467 |
| ATCTCAACTGACTACAGCTC | 149 | sense | −41 | 468 |
| TCTCAACTGACTACAGCTCA | 150 | sense | −42 | 469 |
| CTCAACTGACTACAGCTCAG | 151 | sense | −43 | 470 |
| TCAACTGACTACAGCTCAGG | 152 | sense | −44 | 471 |
| CTACAGCTCAGGGGACTCCC | 160 | sense | −52 | 472 |
| TACAGCTCAGGGGACTCCCA | 161 | sense | −53 | 473 |
| ACAGCTCAGGGGACTCCCAG | 162 | sense | −54 | 474 |
| AGCTCAGGGGACTCCCAGGG | 164 | sense | −56 | 475 |
| GGGGACTCCCAGGGAGCCCA | 170 | sense | −62 | 476 |
| GGGACTCCCAGGGAGCCCAA | 171 | sense | −63 | 477 |
| GGACTCCCAGGGAGCCCAAG | 172 | sense | −64 | 478 |
| GACTCCCAGGGAGCCCAAGG | 173 | sense | −65 | 479 |
| ACTCCCAGGGAGCCCAAGGG | 174 | sense | −66 | 480 |
| GGAGCCCAAGGGGGCTTATC | 182 | sense | −74 | 481 |
| GCCCAAGGGGGCTTATCCGA | 185 | sense | −77 | 482 |
| CCCAAGGGGGCTTATCCGAT | 186 | sense | −78 | 483 |

In certain embodiments, the prime editor is designed using Cas9-SpRY to generate YLY (454-456) FLF mutation using TTC as a phenylalanine codon according to Tables 36-39. The present Example includes pegRNAs characterized by a spacer selected from Table 36, an RT template selected from Table 37, and a PBS sequence selected from Table 38 in the following tables. Information presented in Table 36 additionally includes the strand orientation of the spacer sequence (whether a portion of a sequence that corresponds to the spacer sequence is present in a “sense” or “antisense” strand, e.g., relative to a sequence encoding all or a portion of an EpoR polypeptide), the distance between the spacer sequence and the nearest edited nucleotide of the target sequence, and whether the PAM site is disrupted. The present Example additionally includes secondary nicking sgRNAs characterized by a sequence selected from Table 39.

TABLE 36
Spacers

| | | Distance | | SEQ |
|---|---|---|---|---|
| | | to Edit | Seed/PAM | ID |
| Sequence | Orientation | Start | Disrupt | NO: |
| CCCACCCCACCTAAAGTACC | sense | 0 | 1 | 484 |
| AGTCAGATACCACAAGGTAC | antisense | 0 | 1 | 485 |
| GAGTCAGATACCACAAGGTA | antisense | 1 | 1 | 486 |
| CCCCACCCCACCTAAAGTAC | sense | 1 | 1 | 487 |
| ACCCCACCCCACCTAAAGTA | sense | 2 | 1 | 488 |
| AGAGTCAGATACCACAAGGT | antisense | 2 | 1 | 489 |
| TACCCCACCCCACCTAAAGT | sense | 3 | 0 | 490 |
| CAGAGTCAGATACCACAAGG | antisense | 3 | 0 | 491 |
| CTACCCCACCCCACCTAAAG | sense | 4 | 0 | 492 |
| CCAGAGTCAGATACCACAAG | antisense | 4 | 0 | 493 |
| GCCAGAGTCAGATACCACAA | antisense | 5 | 0 | 494 |
| CCTACCCCACCCCACCTAAA | sense | 5 | 0 | 495 |
| TGCCAGAGTCAGATACCACA | antisense | 6 | 0 | 496 |
| CCCTACCCCACCCCACCTAA | sense | 6 | 0 | 497 |
| ATGCCAGAGTCAGATACCAC | antisense | 7 | 0 | 498 |
| CCCCTACCCCACCCCACCTA | sense | 7 | 0 | 499 |
| CCCCCTACCCCACCCCACCT | sense | 8 | 0 | 500 |
| GATGCCAGAGTCAGATACCA | antisense | 8 | 0 | 501 |
| AGATGCCAGAGTCAGATACC | antisense | 9 | 0 | 502 |
| GCCCCCTACCCCACCCCACC | sense | 9 | 0 | 503 |
| TGCCCCCTACCCCACCCCAC | sense | 10 | 0 | 504 |
| GAGATGCCAGAGTCAGATAC | antisense | 10 | 0 | 505 |
| TGAGATGCCAGAGTCAGATA | antisense | 11 | 0 | 506 |
| CTGCCCCCTACCCCACCCCA | sense | 11 | 0 | 507 |
| GCTGCCCCCTACCCCACCCC | sense | 12 | 0 | 508 |
| TTGAGATGCCAGAGTCAGAT | antisense | 12 | 0 | 509 |
| GTTGAGATGCCAGAGTCAGA | antisense | 13 | 0 | 510 |
| AGCTGCCCCCTACCCCACCC | sense | 13 | 0 | 511 |
| GAGCTGCCCCCTACCCCACC | sense | 14 | 0 | 512 |
| AGTTGAGATGCCAGAGTCAG | antisense | 14 | 0 | 513 |

TABLE 37
RT Templates

| | | SEQ ID |
|---|---|---|
| Sequence | Length | NO: |
| AGGAACAGGA | 10 | 514 |
| AAGGAACAGGA | 11 | 515 |
| CAAGGAACAGGA | 12 | 516 |
| ACAAGGAACAGGA | 13 | 517 |
| CACAAGGAACAGGA | 14 | 518 |
| CCACAAGGAACAGGA | 15 | 519 |
| ACCACAAGGAACAGGA | 16 | 520 |
| TACCACAAGGAACAGGA | 17 | 521 |

TABLE 38
PBS Sequences

| | | SEQ ID |
|---|---|---|
| Sequence | Length | NO: |
| ACTTTAGG | 8 | 522 |
| ACTTTAGGT | 9 | 523 |
| ACTTTAGGTG | 10 | 524 |
| ACTTTAGGTGG | 11 | 525 |
| ACTTTAGGTGGG | 12 | 526 |
| ACTTTAGGTGGGG | 13 | 527 |
| ACTTTAGGTGGGGT | 14 | 528 |
| ACTTTAGGTGGGGTG | 15 | 529 |
| ACTTTAGGTGGGGTGG | 16 | 530 |
| ACTTTAGGTGGGGTGGG | 17 | 531 |

TABLE 39
Secondary Nicking sgRNA Sequences

| | | | Distance | |
|---|---|---|---|---|
| | | | to | |
| | Nick | | pegRNA | SEQ ID |
| Sequence | Position | Orientation | Nick | NO: |
| TACCACAAGGAACAGGAACT | 102 | antisense | 0 | 532 |
| ATACCACAAGGAACAGGAAC | 103 | antisense | 1 | 533 |
| ACCACAAGGAACAGGAACTT | 101 | antisense | −1 | 534 |
| GATACCACAAGGAACAGGAA | 104 | antisense | 2 | 535 |
| CCACAAGGAACAGGAACTTT | 100 | antisense | −2 | 536 |
| AGATACCACAAGGAACAGGA | 105 | antisense | 3 | 537 |
| CACAAGGAACAGGAACTTTA | 99 | antisense | −3 | 538 |
| ACAAGGAACAGGAACTTTAG | 98 | antisense | −4 | 539 |
| CAGATACCACAAGGAACAGG | 106 | antisense | 4 | 540 |
| TCAGATACCACAAGGAACAG | 107 | antisense | 5 | 541 |
| CAAGGAACAGGAACTTTAGG | 97 | antisense | −5 | 542 |
| GTCAGATACCACAAGGAACA | 108 | antisense | 6 | 543 |
| AAGGAACAGGAACTTTAGGT | 96 | antisense | −6 | 544 |
| AGTCAGATACCACAAGGAAC | 109 | antisense | 7 | 545 |
| GAGTCAGATACCACAAGGAA | 110 | antisense | 8 | 546 |
| AGAGTCAGATACCACAAGGA | 111 | antisense | 9 | 547 |
| GCTCCCTGGGAGTCCCCTGA | 152 | antisense | 50 | 548 |
| CTCCCTGGGAGTCCCCTGAG | 151 | antisense | 49 | 549 |
| GGCTCCCTGGGAGTCCCCTG | 153 | antisense | 51 | 550 |
| TCCCTGGGAGTCCCCTGAGC | 150 | antisense | 48 | 551 |
| GGGCTCCCTGGGAGTCCCCT | 154 | antisense | 52 | 552 |
| TGGGCTCCCTGGGAGTCCCC | 155 | antisense | 53 | 553 |
| CCCTGGGAGTCCCCTGAGCT | 149 | antisense | 47 | 554 |
| TTGGGCTCCCTGGGAGTCCC | 156 | antisense | 54 | 555 |
| CCTGGGAGTCCCCTGAGCTG | 148 | antisense | 46 | 556 |
| CTTGGGCTCCCTGGGAGTCC | 157 | antisense | 55 | 557 |
| CTGGGAGTCCCCTGAGCTGT | 147 | antisense | 45 | 558 |
| TGGGAGTCCCCTGAGCTGTA | 146 | antisense | 44 | 559 |
| CCTTGGGCTCCCTGGGAGTC | 158 | antisense | 56 | 560 |
| CCCTTGGGCTCCCTGGGAGT | 159 | antisense | 57 | 561 |
| GGGAGTCCCCTGAGCTGTAG | 145 | antisense | 43 | 562 |
| GGAGTCCCCTGAGCTGTAGT | 144 | antisense | 42 | 563 |
| CCCCTTGGGCTCCCTGGGAG | 160 | antisense | 58 | 564 |
| GAGTCCCCTGAGCTGTAGTC | 143 | antisense | 41 | 565 |
| CCCCCTTGGGCTCCCTGGGA | 161 | antisense | 59 | 566 |
| GCCCCCTTGGGCTCCCTGGG | 162 | antisense | 60 | 567 |
| AGTCCCCTGAGCTGTAGTCA | 142 | antisense | 40 | 568 |
| AGCCCCCTTGGGCTCCCTGG | 163 | antisense | 61 | 569 |
| AAGCCCCCTTGGGCTCCCTG | 164 | antisense | 62 | 570 |
| TAAGCCCCCTTGGGCTCCCT | 165 | antisense | 63 | 571 |
| ATAAGCCCCCTTGGGCTCCC | 166 | antisense | 64 | 572 |
| GATAAGCCCCCTTGGGCTCC | 167 | antisense | 65 | 573 |
| GGATAAGCCCCCTTGGGCTC | 168 | antisense | 66 | 574 |
| CGGATAAGCCCCCTTGGGCT | 169 | antisense | 67 | 575 |
| TCGGATAAGCCCCCTTGGGC | 170 | antisense | 68 | 576 |
| ATCGGATAAGCCCCCTTGGG | 171 | antisense | 69 | 577 |
| CATCGGATAAGCCCCCTTGG | 172 | antisense | 70 | 578 |
| CCATCGGATAAGCCCCCTTG | 173 | antisense | 71 | 579 |
| GCCATCGGATAAGCCCCCTT | 174 | antisense | 72 | 580 |
| GGCCATCGGATAAGCCCCCT | 175 | antisense | 73 | 581 |
| GGGCCATCGGATAAGCCCCC | 176 | antisense | 74 | 582 |
| GGGGCCATCGGATAAGCCCC | 177 | antisense | 75 | 583 |
| AGGGGCCATCGGATAAGCCC | 178 | antisense | 76 | 584 |
| TAGGGGCCATCGGATAAGCC | 179 | antisense | 77 | 585 |
| GTAGGGGCCATCGGATAAGC | 180 | antisense | 78 | 586 |
| AGTAGGGGCCATCGGATAAG | 181 | antisense | 79 | 587 |
| GAGTAGGGGCCATCGGATAA | 182 | antisense | 80 | 588 |
| GGAGTAGGGGCCATCGGATA | 183 | antisense | 81 | 589 |
| TGGAGTAGGGGCCATCGGAT | 184 | antisense | 82 | 590 |
| TTGGAGTAGGGGCCATCGGA | 185 | antisense | 83 | 591 |
| GTTGGAGTAGGGGCCATCGG | 186 | antisense | 84 | 592 |
| GGTTGGAGTAGGGGCCATCG | 187 | antisense | 85 | 593 |
| GGGTTGGAGTAGGGGCCATC | 188 | antisense | 86 | 594 |
| GGCAGCTCAGGGCACAGTGT | 62 | antisense | −40 | 595 |
| GCAGCTCAGGGCACAGTGTC | 61 | antisense | −41 | 596 |
| CAGCTCAGGGCACAGTGTCC | 60 | antisense | −42 | 597 |
| AGCTCAGGGCACAGTGTCCA | 59 | antisense | −43 | 598 |
| GCTCAGGGCACAGTGTCCAT | 58 | antisense | −44 | 599 |
| CTCAGGGCACAGTGTCCATG | 57 | antisense | −45 | 600 |
| TCAGGGCACAGTGTCCATGG | 56 | antisense | −46 | 601 |
| CAGGGCACAGTGTCCATGGA | 55 | antisense | −47 | 602 |
| AGGGCACAGTGTCCATGGAC | 54 | antisense | −48 | 603 |
| GGGCACAGTGTCCATGGACG | 53 | antisense | −49 | 604 |
| GGCACAGTGTCCATGGACGC | 52 | antisense | −50 | 605 |
| GCACAGTGTCCATGGACGCA | 51 | antisense | −51 | 606 |
| CACAGTGTCCATGGACGCAA | 50 | antisense | −52 | 607 |
| ACAGTGTCCATGGACGCAAG | 49 | antisense | −53 | 608 |
| CAGTGTCCATGGACGCAAGA | 48 | antisense | −54 | 609 |
| AGTGTCCATGGACGCAAGAG | 47 | antisense | −55 | 610 |
| GTGTCCATGGACGCAAGAGC | 46 | antisense | −56 | 611 |
| TGTCCATGGACGCAAGAGCT | 45 | antisense | −57 | 612 |
| GTCCATGGACGCAAGAGCTG | 44 | antisense | −58 | 613 |
| TCCATGGACGCAAGAGCTGG | 43 | antisense | −59 | 614 |
| CCATGGACGCAAGAGCTGGG | 42 | antisense | −60 | 615 |
| CATGGACGCAAGAGCTGGGA | 41 | antisense | −61 | 616 |
| ATGGACGCAAGAGCTGGGAG | 40 | antisense | −62 | 617 |
| TGGACGCAAGAGCTGGGAGC | 39 | antisense | −63 | 618 |
| GGACGCAAGAGCTGGGAGCT | 38 | antisense | −64 | 619 |
| GACGCAAGAGCTGGGAGCTG | 37 | antisense | −65 | 620 |
| ACGCAAGAGCTGGGAGCTGG | 36 | antisense | −66 | 621 |
| CGCAAGAGCTGGGAGCTGGG | 35 | antisense | −67 | 622 |
| GCAAGAGCTGGGAGCTGGGG | 34 | antisense | −68 | 623 |
| CAAGAGCTGGGAGCTGGGGT | 33 | antisense | −69 | 624 |
| AAGAGCTGGGAGCTGGGGTC | 32 | antisense | −70 | 625 |
| AGAGCTGGGAGCTGGGGTCC | 31 | antisense | −71 | 626 |
| GAGCTGGGAGCTGGGGTCCA | 30 | antisense | −72 | 627 |
| AGCTGGGAGCTGGGGTCCAG | 29 | antisense | −73 | 628 |
| GCTGGGAGCTGGGGTCCAGG | 28 | antisense | −74 | 629 |
| CTGGGAGCTGGGGTCCAGGA | 27 | antisense | −75 | 630 |
| TGGGAGCTGGGGTCCAGGAT | 26 | antisense | −76 | 631 |
| GGGAGCTGGGGTCCAGGATA | 25 | antisense | −77 | 632 |
| GGAGCTGGGGTCCAGGATAG | 24 | antisense | −78 | 633 |
| GAGCTGGGGTCCAGGATAGT | 23 | antisense | −79 | 634 |
| AGCTGGGGTCCAGGATAGTG | 22 | antisense | −80 | 635 |
| GCTGGGGTCCAGGATAGTGT | 21 | antisense | −81 | 636 |
| CTGGGGTCCAGGATAGTGTA | 20 | antisense | −82 | 637 |
| TGGGGTCCAGGATAGTGTAC | 19 | antisense | −83 | 638 |
| GGGGTCCAGGATAGTGTACT | 18 | antisense | −84 | 639 |
| GGGTCCAGGATAGTGTACTC | 17 | antisense | −85 | 640 |
| GGTCCAGGATAGTGTACTCA | 16 | antisense | −86 | 641 |
| GTCCAGGATAGTGTACTCAA | 15 | antisense | −87 | 642 |
| TCCAGGATAGTGTACTCAAA | 14 | antisense | −88 | 643 |
| CCAGGATAGTGTACTCAAAG | 13 | antisense | −89 | 644 |
| CAGGATAGTGTACTCAAAGC | 12 | antisense | −90 | 645 |
| AGGATAGTGTACTCAAAGCT | 11 | antisense | −91 | 646 |
| GGATAGTGTACTCAAAGCTG | 10 | antisense | −92 | 647 |
| GATAGTGTACTCAAAGCTGG | 9 | antisense | −93 | 648 |
| ATAGTGTACTCAAAGCTGGC | 8 | antisense | −94 | 649 |
| TAGTGTACTCAAAGCTGGCA | 7 | antisense | −95 | 650 |
| AGTGTACTCAAAGCTGGCAG | 6 | antisense | −96 | 651 |

### Example 5

The present Example includes use of base editing to modify an endogenous nucleic acid sequence that encodes EpoR to produce a modified nucleic acid that encodes signaling-enhanced EpoR. As in Example 4, the present Example includes modification of Y454 and Y456. Exemplary adenine base editor reagents for producing a Y454C/Y456C modification can include:

CRISPR-Cas orthologue: Cas9-SpRY
CRISPR Target (5′ to 3′):
(SEQ ID NO: 654)
AAAGTACCTGTACCTTGTGGTAT
Underline: PAM site
Bold: Editing window
Editing outcome: Y454C/Y456C

### Example 6

The present Example includes use of base editing to modify an endogenous nucleic acid sequence that encodes EpoR to produce a modified nucleic acid that encodes signaling-enhanced EpoR. In particular, the present Example includes truncation of EpoR by converting Q474 into a TAG stop codon using a CBE. Exemplary cytosine base editor reagents for producing a stop codon at codon 474 are provided below:

CRISPR-Cas orthologue: SpCas9
CRISPR Target (5′ to 3′):
(SEQ ID NO: 655)
GACTCCCAGGGAGCCCAAGG
Underline: PAM site
Bold: Editing window
Editing outcome: Q474Ter

### Example 7

The present Example includes use of base editing to modify an endogenous nucleic acid sequence that encodes EpoR to produce a modified nucleic acid that encodes signaling-enhanced EpoR. In particular, the present Example includes truncation of EpoR by converting Q477 into a TAA stop codon using a CBE. Exemplary cytosine base editor reagents for producing a stop codon at codon 477 are provided below:

CRISPR-Cas orthologue: SpCas9
CRISPR Target (5′ to 3′):
(SEQ ID NO: 656)
GGAGCCCAAGGGGGCTTATCCGATG
Underline: PAM site
Bold: Editing window
Editing outcome: Q477Ter with bystander mutations potentially introducing A476V

### Example 8

The present Example includes use of base editing to modify an endogenous nucleic acid sequence that encodes EpoR to produce a modified nucleic acid that encodes signaling-enhanced EpoR. In particular, the present Example includes truncation of EpoR by converting the codon for Trp439 into a stop codon using a cytosine base editor (CBE), which permits C: G-to-T: A base changes necessary for making one of the AT-rich stop codon (TAA, TAG, TGA). While those of skill in the art will appreciate that various base editors are available, the present Example utilizes an exemplary CBE that corresponds to BE4max described in Koblan et al., Nat Biotechnol, 36 (9): 843-846 (2018). An episomal plasmid vector (pCBE) was constructed to encode the CBE fused to a C-terminal P2A-EGFP, which acts as a reporter of CBE expression, and under the control of a CMV promoter and a bovine growth hormone (BGH) polyadenylation signal.

Guide RNA sequences were engineered for base editing of a stop codon in the final exon of the endogenous EpoR gene loci (NCBI accession no. NG_021395.1). In particular, the guide RNA sequences were engineered to generate a Trp439Ter mutation. Additionally, a non-targeting control guide (NTG; AAATGTGAGATCAGAGTAAT (SEQ ID NO: 667); Thermo Fisher Scientific) was used as a negative control. Table 40 provides information on the tested guides. Guides sequences were synthesized as duplexed oligonucleotides (Integrated DNA Technologies) with overhangs for cloning and each guide sequence was cloned into a single guide RNA (sgRNA) expression plasmid at a BsmBI restriction site. The sgRNA expression plasmid expresses the cloned guide from a U6 promoter.

TABLE 40
Guide Sequences

| | | | | | Position | |
|---|---|---|---|---|---|---|
| | | SEQ | | | (relative | Truncation |
| | | ID | | Editing | to CDS | size (amino |
| Name | Guide Sequence | NO: | Strand | Window | in exon) | acids) |
| Guide 1 | TGTCCATGGACGCAAGAGCT | 657 | — | CCATG | 383 | 70 |
| Guide 2 | GTGTCCATGGACGCAAGAGC | 658 | — | TCCAT | 384 | 70 |

Underlined sequence indicates nucleotides targeted for base editing.

To modify an endogenous EpoR-encoding nucleic acid, 2×10⁶ HUDEP-2 cells were transfected by nucleofection (Lonza, P3 Primary Cell 4D-Nucleofector Transfection Reagent, V4XP-3032) with (i) 0.25 ug pCBE and (ii) 0.25 ug of one of the two plasmids encoding an EpoR targeting sgRNA or the NTG sgRNA described above. Each condition was performed in triplicate. The transfected cells were cultured in HUDEP-2 culture media (StemSpan SFEM (STEMCELL Technologies, 09650) with 1 μM dexamethasone, 1 μg/mL doxycycline, 50 ng/mL stem cell factor, and 3 U/mL erythropoietin (Epo)). 24 hours post-transfection, dead cells were removed by magnetic-activated cell sorting (MACS) and the cells were returned to culture in HUDEP-2 culture media with 3 U/mL Epo.

To assess the sensitivity of base-edited cells to Epo, 4 days post-transfection, the transfected cells were passaged into HUDEP-2 culture media containing a reduced concentration of Epo (1U Epo/mL) at a concentration of 0.2×10⁶ cells/mL of media. Cells were maintained in culture at a concentration between 0.1×10⁶ cells/mL and 1×10⁶ cells/mL. Aliquots of the cells were harvested for DNA extraction at 4, 18, 20, 25, 30, and 35 days post-transfection. To quantify the level of base editing at each time point, sequencing was performed on the extracted DNA. DNA was purified from HUDEP-2 cells using the Quick-DNA HMW MagBead Kit (Zymo Research, D6060), following manufacturer's instructions. 20 ng of the purified DNA was used in first round PCR with primers specific for the EpoR gene locus with partial Illumina adapters. Table 41 contains the gene-specific sequences of the primers. The first-round PCR products were purified using Ampure beads and 10% of the purified PCR product was used to generate a final sequencing library in a second round PCR to complete the Illumina adapters and indexing sequences. Final libraries were purified using Ampure beads, quantified with the Qubit Fluorometer, and run on a gel to confirm the size. Libraries were pooled equimolar and sequenced on an Illumina MiSeq instrument using 151 bp paired-end sequencing.

TABLE 41
Gene-Specific Sequences of PCR Primers

| | | SEQ ID |
|---|---|---|
| Name | Sequence | NO: |
| Primer-forward | GCTCATCTGCTTTGGCCTCGAAG | 659 |
| Primer-reverse | GCGGCTGGGATAAGGCTGTTCTC | 660 |

The sequencing reads were demultiplexed by sample and aligned to the human reference genome (hg38). Reads with low quality scores were filtered out. Reads that aligned to the designated EpoR amplicon were retained. The number of reads containing the wild type sequence and the number of reads containing a base edit were each counted. The percentage of base edited alleles in each sample was calculated as the total number of reads with a C to T conversion in the guide-editing window divided by the total number of aligned reads in the same window.

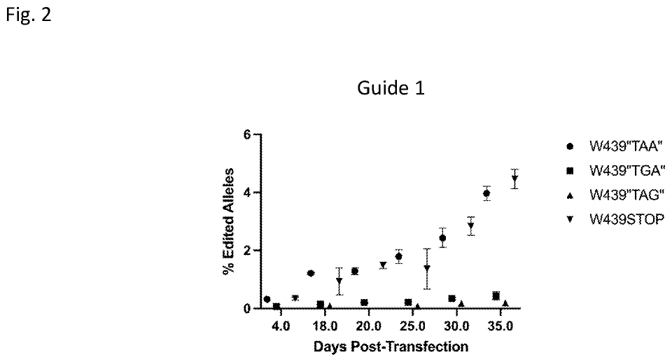

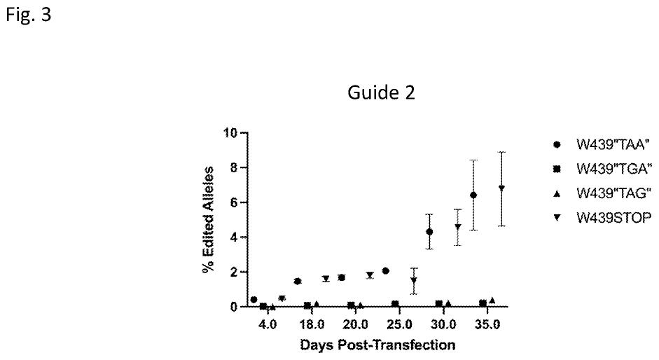

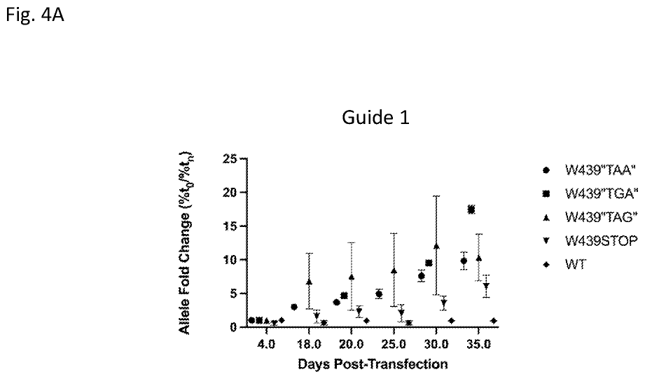

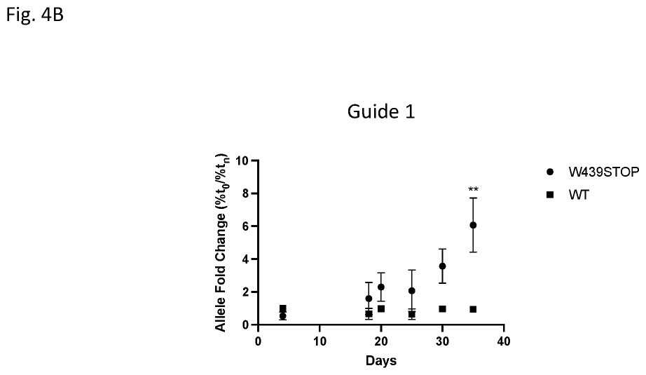

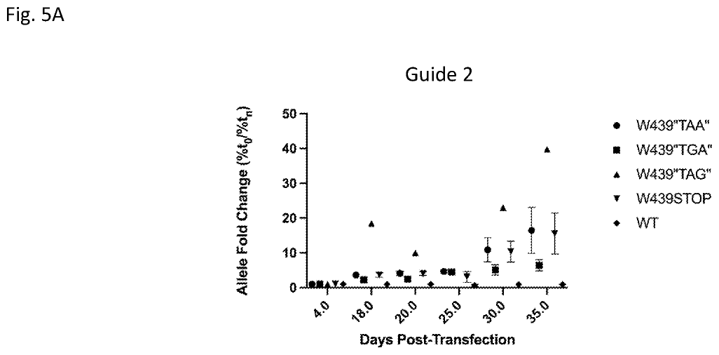

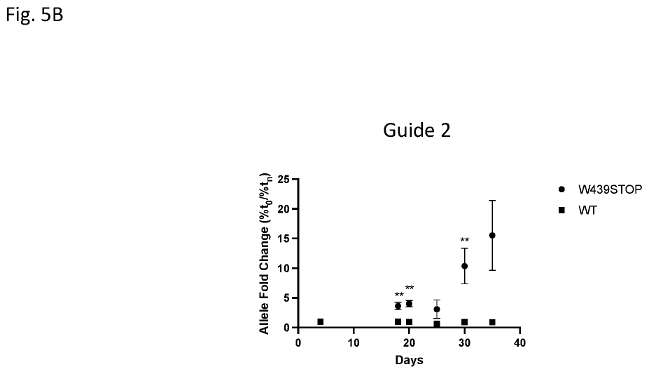

The percentage of base edited alleles at each time point for Guides 1 and 2 are shown in FIGS. 2 and 3, respectively. Results are presented for each of three stop codons generated by base editing (“TAA”, “TGA”, and “TAG”) and combined (“STOP” or “Ter”). The fold change, normalized to the 4 days post-transfection sample, is shown for Guides 1 and 2 in FIGS. 4 and 5, respectively. Fold change values greater than 1 represent an increase in the population of cells with the desired base editing and indicates a growth advantage for the base-edited cells expressing EpoR with the W439Ter mutation. Table 42 summarizes results for each of the predominant stop codon alleles generated by base editing. Analysis of sequencing reads from samples transfected with non-targeting control guide did not show evidence of base editing or generation of an EpoR truncation mutation demonstrating that the base editing observed for Guides 1 and 2 are guide-specific. These results indicate that successful base editing to modify an endogenous nucleic acid sequence that encodes EpoR to produce a modified nucleic acid that encodes signaling-enhanced EpoR

TABLE 42
Results of Base Editing

| | | | | Average | |
|---|---|---|---|---|---|
| | | | SEQ | Percentage | Average |
| | | | ID | of Final | Final Fold |
| Guide | Mutation | Edited Sequence | NO: | Population | Change |
| Guide 1 | W439”TAA” | GCAGCTCAGGGCACAGTG | 661 | 3.97 | 9.84 |
| | | TTTATGGACGCAAGAGCT | | | |
| Guide 1 | W439”TAG” | GCAGCTCAGGGCACAGTG | 662 | 0.19 | 10.3* |
| | | TCTATGGACGCAAGAGCT | | | |
| Guide 1 | W439”TGA” | GCAGCTCAGGGCACAGTG | 663 | 0.43 | 17.5 |
| | | TTCATGGACGCAAGAGCT | | | |
| Guide 2 | W439”TAA” | GCAGCTCAGGGCACAGTG | 664 | 6.42 | 16.5 |
| | | TTTATGGACGCAAGAGCT | | | |
| Guide 2 | W439”TAG” | GCAGCTCAGGGCACAGTG | 665 | 0.40 | 39.8* |
| | | TCTATGGACGCAAGAGCT | | | |
| Guide 2 | W439”TGA” | GCAGCTCAGGGCACAGTG | 666 | 0.21 | 6.38 |
| | | TTCATGGACGCAAGAGCT | | | |

Final: day 35 post-transfection; Fold change relative to day 4 post-transfection.
*Values reflective of only n = 1 replicates due to low base editing rates for these alleles at initial time points.

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. For example, elements of claims that depend directly or indirectly from a certain independent claim presented herein serve as support for those elements being presented in additional dependent claims of one or more other independent claims. Throughout the description, where compositions or methods are described as having, including, or comprising specific elements, compositions that consist essentially of, consist of, or do not comprise the recited elements. All references cited herein are hereby incorporated by reference.

What is claimed is:

1. A method comprising contacting one or more cells of a mammalian subject with (i) an editing enzyme that modifies an endogenous erythropoietin receptor (EpoR)-encoding nucleic acid to produce a modified EpoR-encoding nucleic acid, wherein the modified EpoR-encoding nucleic acid encodes a signaling-enhanced EpoR polypeptide, or (ii) a transgene that encodes a signaling-enhanced EpoR polypeptide,
wherein the contacting occurs in vivo, in vitro, or ex vivo.

2. Use of (i) an editing enzyme for modification of an endogenous erythropoietin receptor (EpoR)-encoding nucleic acid of one or more cells of a mammalian subject to produce a modified EpoR-encoding nucleic acid, wherein the modified EpoR-encoding nucleic acid encodes a signaling-enhanced EpoR polypeptide or (ii) a transgene that encodes a signaling-enhanced EpoR polypeptide,
wherein the use comprises contacting one or more cells of a mammalian subject with the editing enzyme or transgene in vivo, in vitro, or ex vivo.

3. The method of claim 1 or use of claim 2, wherein the signaling-enhanced EpoR polypeptide comprises a C-terminal truncation corresponding to truncation of amino acids 30 to 130 of SEQ ID NO: 1.

4. The method of claim 1 or use of claim 2, wherein the signaling-enhanced EpoR polypeptide comprises a C-terminal truncation corresponding to truncation of 42 amino acids of SEQ ID NO: 1.

5. The method of claim 1 or use of claim 2, wherein the signaling-enhanced EpoR polypeptide comprises substitution or deletion of one or more amino acids corresponding to Tyr426, Tyr454, and Tyr456 of SEQ ID NO: 1.

6. The method of claim 1 or use of claim 2, wherein the signaling-enhanced EpoR polypeptide comprises one or more of Pro381 Gln fs*2, Phe424*, Leu 429 Trp fs*24, Glu 399*, Ser 415 His fs*18, Ser 412 Arg fs*41, Ser 412*, Glu 417*, Ile428 Tyr fs*17, Asp430 Glu fs*26, Ser432 Gly fs*15, Asp430 Gly fs*15, Gln434 Cys fs*17, Gln434*, Trp439*, Trp439*, Gln434 Pro fs *11, Pro438 Met fs*6, Gly418 Pro fs *34, Glu425*, Tyr454*, and/or Tyr426*.

7. The method of claim 1 or use of claim 2, wherein the signaling-enhanced EpoR polypeptide comprises one or more of Pro381_Ser382delinsGln*, p.Ser382*, Leu 452*, Ser401*, Ser407*, Tyr454*, Tyr456*, Trp439*, Asp467*, Tyr468*, Tyr454Phe, Tyr426Phe; Tyr454Phe; Tyr456Phe, Tyr454Phe; Tyr456Phe, Tyr426Phe, Tyr456Phe, Tyr426Phe; Tyr456Phe, Tyr426Phe; Tyr454Phe, Tyr426Cys; Tyr454Cys; Tyr456Cys, Tyr454Cys; Tyr456Cys, Tyr426Cys; Tyr454Cys, Tyr426Cys; Tyr456Cys, Tyr426Cys, Tyr454Cys, Tyr456Cys, Gln474*, Gln477*, and/or Tyr426_Tyr456del.

8. The method or use of any one of claims 1-7, wherein the modified EpoR-encoding nucleic acid or transgene comprises a nucleic acid modification selected from Table 1.

9. The method or use of any one of claims 1-8, wherein the contacting occurs in vivo.

10. The method or use of any one of claims 1-9, comprising administering to the mammalian subject a nucleic acid encoding the editing enzyme or transgene.

11. The method or use of claim 10, wherein the nucleic acid encoding the editing enzyme further encodes a guide RNA that directs editing of the endogenous erythropoietin receptor (EpoR)-encoding nucleic acid by the editing enzyme.

12. The method or use of claim 10 or claim 11, wherein the nucleic acid encoding the editing enzyme or transgene is administered parenterally.

13. The method or use of any one of claims 10-12, wherein the nucleic acid encoding the editing enzyme or transgene is administered by injection.

14. The method or use of any one of claims 10-13, wherein the nucleic acid encoding the editing enzyme or transgene is administered intravenously.

15. The method or use of claim any one of claims 1-14, comprising mobilization of hematopoietic stem cells of the subject prior to administration of the nucleic acid.

16. The method or use of any one of claims 1-15, comprising administering one or more immunosuppression agents to the subject, optionally wherein the administration of the one or more immunosuppression agents is prior to the administration of the nucleic acid.

17. The method or use of any one of claims 1-16, wherein the nucleic acid encoding the editing enzyme or transgene comprises a transgene encoding an inhibitor-resistant MGMT polypeptide, optionally wherein the inhibitor-resistant MGMT polypeptide is MGMTP140K.

18. The method or use of claim 17, comprising administering one or more MGMT inhibitors to the subject after the nucleic acid has been administered.

19. The method or use of claim 18, wherein the one or more MGMT inhibitors comprises O6BG or an analog or derivative thereof, and/or wherein the one or more MGMT inhibitors comprises Lomeguatrib.

20. The method or use of any one of claims 17-19, comprising administering one or more alkylating agents to the subject after the nucleic acid has been administered.

21. The method or use of claim 20, wherein the one or more alkylating agent comprises 1,3-bis(2-chloroethyl)-1-nitrosourea (BCNU) or temozolomide.

22. The method or use of any one of claims 1-21, wherein the modified EpoR-encoding nucleic acid confers a competitive advantage to cells comprising the modified EpoR-encoding nucleic acid.

23. A nucleic acid encoding (i) an editing enzyme that modifies an endogenous erythropoietin receptor (EpoR)-encoding nucleic acid to produce a modified EpoR-encoding nucleic acid, wherein the modified EpoR-encoding nucleic acid encodes a signaling-enhanced EpoR polypeptide, and optionally a guide RNA, or (ii) a transgene that encodes a signaling-enhanced EpoR polypeptide, and optionally (iii) a nucleic acid sequence encoding a therapeutic agent.

24. The nucleic acid of claim 23, wherein the signaling-enhanced EpoR polypeptide comprises a C-terminal truncation corresponding to truncation of amino acids 30 to 130 of SEQ ID NO: 1.

25. The nucleic acid of claim 23, wherein the signaling-enhanced EpoR polypeptide comprises a C-terminal truncation corresponding to truncation of 42 amino acids of SEQ ID NO: 1.

26. The nucleic acid of claim 23, wherein the signaling-enhanced EpoR polypeptide comprises substitution or deletion of one or more amino acids corresponding to Tyr426, Tyr454, and Tyr456 of SEQ ID NO: 1.

27. The nucleic acid of claim 23, wherein the signaling-enhanced EpoR polypeptide comprises one or more of Pro381 Gln fs*2, Phe424*, Leu 429 Trp fs*24, Glu 399*, Ser 415 His fs*18, Ser 412 Arg fs*41, Ser 412*, Glu 417*, Ile428 Tyr fs*17, Asp430 Glu fs*26, Ser432 Gly fs* 15, Asp430 Gly fs*15, Gln434 Cys fs*17, Gln434*, Trp439*, Trp439*, Gln434 Pro fs*11, Pro438 Met fs*6, Gly418 Pro fs *34, Glu425*, Tyr454*, and/or Tyr426*.

28. The nucleic acid of claim 23, wherein the signaling-enhanced EpoR polypeptide comprises one or more of Pro381_Ser382delinsGln*, p.Ser382*, Leu 452*, Ser401*, Ser407*, Tyr454*, Tyr456*, Trp439*, Asp467*, Tyr468*, Tyr454Phe, Tyr426Phe; Tyr454Phe; Tyr456Phe, Tyr454Phe; Tyr456Phe, Tyr426Phe, Tyr456Phe, Tyr426Phe; Tyr456Phe, Tyr426Phe; Tyr454Phe, Tyr426Cys; Tyr454Cys; Tyr456Cys, Tyr454Cys; Tyr456Cys, Tyr426Cys; Tyr454Cys, Tyr426Cys; Tyr456Cys, Tyr426Cys, Tyr454Cys, Tyr456Cys, Gln474*, Gln477*, and/or Tyr426_Tyr456del.

29. The nucleic acid of any one of claims 23-28, wherein the modified EpoR-encoding nucleic acid or transgene comprises a nucleic acid modification selected from Table 1.

30. The nucleic acid nucleic acid encoding an editing enzyme of any one of claims 23-29, wherein the nucleic acid encoding the editing enzyme encodes a guide RNA that directs editing of the endogenous erythropoietin receptor (EpoR)-encoding nucleic acid by the editing enzyme.

31. A pharmaceutical composition comprising the nucleic acid encoding an editing enzyme or transgene of any one of claims 23-30.

32. The pharmaceutical composition of claim 31, wherein the pharmaceutical composition is formulated for administration to a mammalian subject, optionally wherein the mammalian subject is a human subject.

33. The pharmaceutical composition of claim 31 or claim 32, wherein the pharmaceutical composition is formulated for parenteral administration.

34. The pharmaceutical composition of any one of claims 31-33, wherein the pharmaceutical composition is formulated for injection.

35. The pharmaceutical composition of any one of claims 31-34, wherein the pharmaceutical composition is formulated for intravenous injection.

36. A kit comprising the nucleic acid encoding an editing enzyme or transgene of any one of claims 23-30 or the pharmaceutical composition of any one of claims 31-35.

37. The kit of claim 36, wherein the kit comprises one or more mobilization agents.

38. The kit of claim 36 or 37, wherein the kit comprises one or more immunosuppression agents.

39. The kit of any one of claims 36-38, wherein the kit comprises instructions for administering a vector comprising the nucleic acid encoding an editing enzyme or transgene to a mammalian subject.

40. The method, use, nucleic acid, pharmaceutical composition, or kit of any one claims 1-39, wherein the editing enzyme is a base editing enzyme that deaminates a nucleobase in the endogenous erythropoietin receptor (EpoR)-encoding nucleic acid.

41. The method, use, nucleic acid, pharmaceutical composition, or kit of claim 40, wherein the base editing enzyme comprises a DNA binding domain and a deaminase domain.

42. The method, use, nucleic acid, pharmaceutical composition, or kit of claim 41, wherein the DNA binding domain and deaminase domain are fused.

43. The method, use, nucleic acid, pharmaceutical composition, or kit of claim 41 or 42, wherein the DNA binding domain is a zinc finger domain.

44. The method, use, nucleic acid, pharmaceutical composition, or kit of claim 41 or 42, wherein the DNA binding domain is a TALEN domain.

45. The method, use, nucleic acid, pharmaceutical composition, or kit of claim 41 or 42, wherein the DNA binding domain is an RNA guided DNA binding domain.

46. The method, use, nucleic acid, pharmaceutical composition, or kit of claim 45, wherein the RNA guided DNA binding domain is a modified Cas9 variant or a modified Cas12a variant.

47. The method, use, nucleic acid, pharmaceutical composition, or kit of claim 45 or 46, wherein the RNA guided DNA binding domain is a catalytically impaired nuclease domain.

48. The method, use, nucleic acid, pharmaceutical composition, or kit of any one of claims 45-47, wherein the RNA guided DNA binding domain is a nickase variant.

49. The method, use, nucleic acid, pharmaceutical composition, or kit of any one of claims 41-48, wherein the deaminase domain is a cytidine deaminase domain.

50. The method, use, nucleic acid, pharmaceutical composition, or kit of any one of claims 41-48, wherein the deaminase domain is an adenosine deaminase domain.

51. The method, use, nucleic acid, pharmaceutical composition, or kit of any one claims 1-39, wherein the editing enzyme is a prime editing enzyme that comprises a DNA binding domain and a reverse transcriptase domain.

52. The method, use, nucleic acid, pharmaceutical composition, or kit of claim 51, wherein the DNA binding domain is an RNA guided DNA binding domain.

53. The method, use, nucleic acid, pharmaceutical composition, or kit of claim 52, wherein the RNA guided DNA binding domain and reverse transcriptase domain are fused.

54. The method, use, nucleic acid, pharmaceutical composition, or kit of claim 52 or 53, wherein the RNA guided DNA binding domain is a modified Cas9 variant or a modified Cas12a variant.

55. The method, use, nucleic acid, pharmaceutical composition, or kit of any one of claims 52-54, wherein the RNA guided DNA binding domain is a catalytically impaired nuclease domain.

56. The method, use, nucleic acid, pharmaceutical composition, or kit of any one of claims 52-55, wherein the RNA guided DNA binding domain is a nickase variant.

57. The method, use, nucleic acid, pharmaceutical composition, or kit of any one of claims 51-56, wherein the reverse transcriptase domain is an MLV reverse transcriptase domain.

58. The method, use, nucleic acid, pharmaceutical composition, or kit of any one claims 1-39, wherein the editing enzyme is an RNA editing enzyme that deaminates a nucleobase in mRNA transcripts produced from the endogenous EpoR gene to produce a modified EpoR mRNA transcript.

59. The method, use, nucleic acid, pharmaceutical composition, or kit of any one of claims 10-58, wherein the nucleic acid encoding the editing enzyme or transgene is encapsidated in a viral particle.

60. The method, use, nucleic acid, pharmaceutical composition, or kit of claim 59, wherein the viral particle is a recombinant adenovirus.

61. The method, use, nucleic acid, pharmaceutical composition, or kit of claim 60, wherein the recombinant adenovirus is a recombinant Ad35 virus.

62. The method, use, nucleic acid, pharmaceutical composition, or kit of any one of claims 10-58, wherein the nucleic acid encoding the editing enzyme is encapsulated in a lipid nanoparticle.

63. The method, use, nucleic acid, pharmaceutical composition, or kit of any one of claims 10-58, wherein the nucleic acid encoding the editing enzyme is encapsulated in a liposome.

64. The method, use, nucleic acid, pharmaceutical composition, or kit of any one of claims 10-58, wherein the nucleic acid further comprises a therapeutic payload.

65. The method, use, nucleic acid, pharmaceutical composition, or kit of claim 64, wherein the therapeutic payload is an integrating payload, optionally wherein the integrating payload does not encode the editing enzyme.

66. The method, use, nucleic acid, pharmaceutical composition, or kit of any one of claims 1-65, wherein the endogenous erythropoietin receptor (EpoR)-encoding nucleic acid is an EpoR gene in the genomes of the one or more cells.

67. The method, use, nucleic acid, pharmaceutical composition, or kit of any one of claims 1-65, wherein the endogenous erythropoietin receptor (EpoR)-encoding nucleic acid is an EpoR mRNA transcript expressed from an EpoR gene of a genome of the one or more cells.

68. The method, use, nucleic acid, pharmaceutical composition, or kit of any one of claims 1-67, wherein the cells are hematopoietic stem cells.

69. The method, use, nucleic acid, pharmaceutical composition, or kit of any one of claims 1-68, wherein the signaling-enhanced EpoR polypeptide comprises a C-terminal MDTVP (SEQ ID NO: 218) amino acid sequence.
